SEQUENCE LISTING (89 sequences)

SEQ ID NO: 1 - DNA, 29 bp, Artificial
  Microarray 29-mer used to assay SNP
attaagcactccacrtgttttctttatag

SEQ ID NO: 2 - DNA, 29 bp, Artificial
  Microarray 29-mer used to assay SNP
taaatatatgctccyatgtttttttccta

SEQ ID NO: 3 - DNA, 29 bp, Artificial
  Microarray 29-mer used to assay SNP
tgtctgagctctcaytcagagacccagga

SEQ ID NO: 4 - DNA, 29 bp, Artificial
  Microarray 29-mer used to assay SNP
acccacaaaagacakagagcgaacaacca

SEQ ID NO: 5 - DNA, 29 bp, Artificial
  Microarray 29-mer used to assay SNP
gaacacttggaactrcacctgggcacctc

SEQ ID NO: 6 - DNA, 29 bp, Artificial
  Microarray 29-mer used to assay SNP
aatagagtaactgaygctttttccaacat

SEQ ID NO: 7 - DNA, 29 bp, Artificial
  Microarray 29-mer used to assay SNP
taccatataaattarcagcagagaccctg

SEQ ID NO: 8 - DNA, 29 bp, Artificial
  Microarray 29-mer used to assay SNP
cctttcctggatacrtgtctactgtaaat

SEQ ID NO: 9 - DNA, 29 bp, Artificial
  Microarray 29-mer used to assay SNP
gctgtggtgcaaagygtcctcctggagag

SEQ ID NO: 10 - DNA, 29 bp, Artificial
  Microarray 29-mer used to assay SNP
gtgaacagcgaggtyattacctgagactg

SEQ ID NO: 11 - DNA, 29 bp, Artificial
  Microarray 29-mer used to assay SNP
ggaggaagccatatsatcatcattggaga

SEQ ID NO: 12 - DNA, 29 bp, Artificial
  Microarray 29-mer used to assay SNP
gccatgggcatgggygctgataagggcca

SEQ ID NO: 13 - DNA, 29 bp, Artificial
  Microarray 29-mer used to assay SNP
cacatgatcgtaggrcattgggatgctct

SEQ ID NO: 14 - DNA, 29 bp, Artificial
  Microarray 29-mer used to assay SNP
tccaactaccacttyggggtcacatatgt

SEQ ID NO: 15 - DNA, 29 bp, Artificial
  Microarray 29-mer used to assay SNP
tggttttgccattcrtcttgctgctgaac

SEQ ID NO: 16 - DNA, 29 bp, Artificial
  Microarray 29-mer used to assay SNP
gcacctggctgggarttagaggtttctaa

SEQ ID NO: 17 - DNA, 29 bp, Artificial
  Microarray 29-mer used to assay SNP
caggtggggccactygctaattctcatgt

SEQ ID NO: 18 - DNA, 29 bp, Artificial
  Microarray 29-mer used to assay SNP
atgggttaggagaarggagcccttgaggg

SEQ ID NO: 19 - DNA, 29 bp, Artificial
  Microarray 29-mer used to assay SNP
taacagaaggtattrattggctatgcact

SEQ ID NO: 20 - DNA, 29 bp, Artificial
  Microarray 29-mer used to assay SNP
ggcttctccccgacrtcagactgtattgt

SEQ ID NO: 21 - DNA, 29 bp, Artificial
  Microarray 29-mer used to assay SNP
gtcctgtgctgtctytcagacgctgtgtc

SEQ ID NO: 22 - DNA, 29 bp, Artificial
  Microarray 29-mer used to assay SNP
tagttttgatcacaytgagtttgaagttc

SEQ ID NO: 23 - DNA, 29 bp, Artificial
  Microarray 29-mer used to assay SNP
aaactgtatgacacytgagagtccaccct

SEQ ID NO: 24 - DNA, 29 bp, Artificial
  Microarray 29-mer used to assay SNP
tagttttgatcacantgagtttgaagttc

SEQ ID NO: 25 - DNA, 29 bp, Artificial
  Microarray 29-mer used to assay SNP
atacacggaatttcnctcacaattgtcca

SEQ ID NO: 26 - DNA, 29 bp, Artificial
  Microarray 29-mer used to assay SNP
ttcaggttattctcntcagctccacaaat

SEQ ID NO: 27 - DNA, 29 bp, Artificial
  Microarray 29-mer used to assay SNP
aggaggagctggacnttctgaaaggaaag

SEQ ID NO: 28 - DNA, 29 bp, Artificial
  Microarray 29-mer used to assay SNP
ggtaggggcactccnctgcctcttcgcat

SEQ ID NO: 29 - DNA, 29 bp, Artificial
  Microarray 29-mer used to assay SNP
aggttacttgaaganaaaatgatctaaat

SEQ ID NO: 30 - DNA, 29 bp, Artificial
  Microarray 29-mer used to assay SNP
aatgcttttgggctnttcaagtaagtgca

SEQ ID NO: 31 - DNA, 29 bp, Artificial
  Microarray 29-mer used to assay SNP
ctctttgattcatgntggataaggcttca

SEQ ID NO: 32 - DNA, 29 bp, Artificial
  Microarray 29-mer used to assay SNP
gtaactgtgtgctcnttgaggacctatct

SEQ ID NO: 33 - DNA, 29 bp, Artificial
  Microarray 29-mer used to assay SNP
atttgacaatgttanacctgtggaaaatt

SEQ ID NO: 34 - DNA, 29 bp, Artificial
  Microarray 29-mer used to assay SNP
aaatgcagagaaggngagtgatctgccta

SEQ ID NO: 35 - DNA, 29 bp, Artificial
  Microarray 29-mer used to assay SNP
gccaatgtcttgaangtgcatagacttgt

SEQ ID NO: 36 - DNA, 29 bp, Artificial
  Microarray 29-mer used to assay SNP
tctagctaaaatacnaaaggctcacactt

SEQ ID NO: 37 - DNA, 29 bp, Artificial
  Microarray 29-mer used to assay SNP
acttctcattagccntcttcctattcttt

SEQ ID NO: 38 - DNA, 29 bp, Artificial
  Microarray 29-mer used to assay SNP
aaatttaggaagacnctttggagttacac

SEQ ID NO: 39 - DNA, 29 bp, Artificial
  Microarray 29-mer used to assay SNP
tgttctagcagagtnaagtcattactggc

SEQ ID NO: 40 - DNA, 29 bp, Artificial
  Microarray 29-mer used to assay SNP
gagctcatgggccangttaaggcgctcag

SEQ ID NO: 41 - DNA, 29 bp, Artificial
  Microarray 29-mer used to assay SNP
tgtgcagggaaggcntcttagtcaagtcc

SEQ ID NO: 42 - DNA, 29 bp, Artificial
  Microarray 29-mer used to assay SNP
acacttccctttccngtaacacatgaaac

SEQ ID NO: 43 - DNA, 29 bp, Artificial
  Microarray 29-mer used to assay SNP
aaaccacagccgttntccataatacaaag

SEQ ID NO: 44 - DNA, 29 bp, Artificial
  Microarray 29-mer used to assay SNP
tggtgcagaagtaangaacaaaacagcca

SEQ ID NO: 45 - DNA, 29 bp, Artificial
  Microarray 29-mer used to assay SNP
ctatactcacacctngtaatgttacccag

SEQ ID NO: 46 - DNA, 29 bp, Artificial
  Microarray 29-mer used to assay SNP
agcactctagaaatnccgtctcaagcagt

SEQ ID NO: 47 - DNA, 29 bp, Artificial
  Microarray 29-mer used to assay SNP
aaggctgtcgtgggncccagtaaggacat

SEQ ID NO: 48 - DNA, 29 bp, Artificial
  Microarray 29-mer used to assay SNP
gacatgcccgtgatnccctcatgcagcct

SEQ ID NO: 49 - DNA, 29 bp, Artificial
  Microarray 29-mer used to assay SNP
gacacaagcttggtnggacctgagtcccc

SEQ ID NO: 50 - DNA, 29 bp, Artificial
  Microarray 29-mer used to assay SNP
ggacctgggtccgcncaccaaggcctggt

SEQ ID NO: 51 - DNA, 29 bp, Artificial
  Microarray 29-mer used to assay SNP
tcaaccactataaancctctctgtgcccg

SEQ ID NO: 52 - DNA, 29 bp, Artificial
  Microarray 29-mer used to assay SNP
tgcaaataagcagangctccatggtctga

SEQ ID NO: 53 - DNA, 29 bp, Artificial
  Microarray 29-mer used to assay SNP
cagcctgcccagttngggagacacaccca

SEQ ID NO: 54 - DNA, 29 bp, Artificial
  Microarray 29-mer used to assay SNP
tggaggaaaccgatnataaaatgataatt

SEQ ID NO: 55 - DNA, 29 bp, Artificial
  Microarray 29-mer used to assay SNP
atatataaggttcanaatcacagcgctcc

SEQ ID NO: 56 - DNA, 29 bp, Artificial
  Microarray 29-mer used to assay SNP
gctaataatgaaganagatcagctgttat

SEQ ID NO: 57 - DNA, 29 bp, Artificial
  Microarray 29-mer used to assay SNP
ggtatatgagacaanaagacagatggggc

SEQ ID NO: 58 - DNA, 29 bp, Artificial
  Microarray 29-mer used to assay SNP
tgtttgtgttggttnggcctggatgccac

SEQ ID NO: 59 - DNA, 29 bp, Artificial
  Microarray 29-mer used to assay SNP
ttggaaaactctacntgcctagtccaagg

SEQ ID NO: 60 - DNA, 29 bp, Artificial
  Microarray 29-mer used to assay SNP
aaagacaaaagtccnagtttatgagaaaa

SEQ ID NO: 61 - DNA, 29 bp, Artificial
  Microarray 29-mer used to assay SNP
ttgagttaggcatanggcgactgggttca

SEQ ID NO: 62 - DNA, 29 bp, Artificial
  Microarray 29-mer used to assay SNP
ttaatatgaaggtanttatactaatgata

SEQ ID NO: 63 - DNA, 29 bp, Artificial
  Microarray 29-mer used to assay SNP
acatgtaaacagttntaatcctgaatgat

SEQ ID NO: 64 - DNA, 29 bp, Artificial
  Microarray 29-mer used to assay SNP
tttcttttaattcanccatctgcacactg

SEQ ID NO: 65 - DNA, 29 bp, Artificial
  Microarray 29-mer used to assay SNP
cagagctctgaccangaaggcccagcagc

SEQ ID NO: 66 - DNA, 29 bp, Artificial
  Microarray 29-mer used to assay SNP
cagactgggtttccntctgcgtacttgcc

SEQ ID NO: 67 - DNA, 29 bp, Artificial
  Microarray 29-mer used to assay SNP
gatgaaatgaaaagntgactctatgtggg

SEQ ID NO: 68 - DNA, 29 bp, Artificial
  Microarray 29-mer used to assay SNP
agtgagccacccacnaactccagggtgaa

SEQ ID NO: 69 - DNA, 29 bp, Artificial
  Microarray 29-mer used to assay SNP
tggaacttcctgggngatgagttgtacga

SEQ ID NO: 70 - DNA, 29 bp, Artificial
  Microarray 29-mer used to assay SNP
tttgggtgggagaanggatttaatgtctg

SEQ ID NO: 71 - DNA, 29 bp, Artificial
  Microarray 29-mer used to assay SNP
ctttaaaatattgangtgcttaatgcaaa

SEQ ID NO: 72 - DNA, 29 bp, Artificial
  Microarray 29-mer used to assay SNP
attaaaggcaagatnatctaaagcacacc

SEQ ID NO: 73 - DNA, 29 bp, Artificial
  Microarray 29-mer used to assay SNP
ctggggtctaaatgnagtagggaggtatg

SEQ ID NO: 74 - DNA, 29 bp, Artificial
  Microarray 29-mer used to assay SNP
atcttgacagagggncattactgtgagcc

SEQ ID NO: 75 - DNA, 29 bp, Artificial
  Microarray 29-mer used to assay SNP
ttccccatctgtacnatggaaatgataaa

SEQ ID NO: 76 - DNA, 29 bp, Artificial
  Microarray 29-mer used to assay SNP
gtctccttctgcccnctctcgctttggcg

SEQ ID NO: 77 - DNA, 29 bp, Artificial
  Microarray 29-mer used to assay SNP
ttcattggggtcctnatcgacctctggaa

SEQ ID NO: 78 - DNA, 29 bp, Artificial
  Microarray 29-mer used to assay SNP
cttcagaatgtactnttcaaaaaaggatg

SEQ ID NO: 79 - DNA, 29 bp, Artificial
  Microarray 29-mer used to assay SNP
ccaggaaggaacccngagccccccaccaa

SEQ ID NO: 80 - DNA, 29 bp, Artificial
  Microarray 29-mer used to assay SNP
ttaatctccacttgncctacctcctaccc

SEQ ID NO: 81 - DNA, 29 bp, Artificial
  Microarray 29-mer used to assay SNP
ggcttctaagaatgnttttcagttcattc

SEQ ID NO: 82 - DNA, 29 bp, Artificial
  Microarray 29-mer used to assay SNP
acacttcagtcttcnctgatggctttttt

SEQ ID NO: 83 - DNA, 29 bp, Artificial
  Microarray 29-mer used to assay SNP
gttgcatccaagggnaagttatagtttgt

SEQ ID NO: 84 - DNA, 29 bp, Artificial
  Microarray 29-mer used to assay SNP
tcattctatcgcccntgttggagtgcagt

SEQ ID NO: 85 - DNA, 29 bp, Artificial
  Microarray 29-mer used to assay SNP
gagggaggtggaacngcacactggacttc

SEQ ID NO: 86 - DNA, 29 bp, Artificial
  Microarray 29-mer used to assay SNP
tgtcttgctcagctntggggctgccactc

SEQ ID NO: 87 - DNA, 29 bp, Artificial
  Microarray 29-mer used to assay SNP
gggacacaggaacgnagacttggacctca

SEQ ID NO: 88 - DNA, 29 bp, Artificial
  Microarray 29-mer used to assay SNP
tgggaccctgggaanccctggcctccagg

SEQ ID NO: 89 - DNA, 29 bp, Artificial
  Microarray 29-mer used to assay SNP
caccctgcccaccanggctccaaagaagc

(19) United States
(12) Patent Application Publication
(10) Pub. No.: US 2006/0228728 A1
(43) Pub. Date: Oct. 12, 2006

(54) Genetic basis of Alzheimer's disease and diagnosis and treatment thereof

(75) Inventors: David R. Cox, Belmont, CA (US); Dennis Ballinger, Menlo Park, CA (US); Erica Beilharz, Menlo Park, CA (US); Karel Konvicka, Palo Alto, CA (US); Laura Stuve, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Perlegen Sciences, Inc., Mountain View, CA (US)

(21) Appl. No.: 11/344,975

(22) Filed: Jan. 31, 2006

Related U.S. Application Data
(60) Provisional application No. 60/648,957, filed on Jan. 31, 2005.

(51) Int. Cl. C12Q 1/68

(52) U.S. Cl. 435/6

(57) ABSTRACT

A collection of polymorphic sites having resistance or susceptibility to Alzheimer's disease is provided. The sites are useful in methods of diagnosing and treating Alzheimer's disease and related conditions.

### CROSS-REFERENCES TO RELATED APPLICATIONS

The present application is a nonprovisional of and claims the benefit of U.S. Ser. No. 60/648,957, filed Jan. 31, 2005, which is incorporated by reference in its entirety for all purposes.

### STATEMENT AS TO RIGHTS TO INVENTIONS MADE UNDER FEDERALLY SPONSORED RESEARCH OR DEVELOPMENT

This invention was made with the support of the United States government under Contract number 1R44AG02402701 by National Institute of Aging/National Institute of Health.

### BACKGROUND OF THE INVENTION

Alzheimer's disease (AD) is a progressive disorder that gradually destroys a person's brain, including the brain's memory ability and ability to learn, reasoning, judgment, communication and ability to carry out daily activities. As AD progresses, individuals may also experience changes in personality and behavior, such as anxiety, depression, suspiciousness or agitation, infantile-like behavior, as well as delusions or hallucinations. The duration of the illness may vary from individual to individual. A person suffering from AD eventually requires complete care. If that individual does not die from other serious illness, complications from the AD and the loss of brain function itself can cause death.

Drugs such as tacrine (Cognex), donepezil (Aricept), rivastigmine (Exelon), or galantamine (Reminyl) have been reported to help people in the early and middle stages of the disease or delay some symptoms. Another drug, memantine (Namenda), has been approved for treatment of moderate to severe AD. It has also been reported that non-inflammatory drugs such as nonsteroidal anti-inflammatory drugs (NSAIDs) help slow the progression of AD. Vitamin E has also been reported to slow the progress of AD.

### BRIEF SUMMARY OF THE CLAIMED INVENTION

The invention provides methods of polymorphic profiling. Such methods determine a polymorphic profile in at least two but no more than 1000 different haplotype blocks, at least two of the haplotype blocks including a gene selected from the group consisting of APOE, APP, APOC1, CACNA1C, CKM, FARS1, KIAA1486, LOC147468, LOC166522, MGC39715, NCE2, PCBP3, PDE11A, PFKFB2, PVRL2, SEC13L1, TOMM40 and TTLL2. Preferably, the at least two haplotype blocks in which the polymorphic profile is determined at polymorphic sites in or within 10 kb of the at least two genes selected from the group. Optionally, the method further comprises determining the total number of resistance and susceptibility alleles in the polymorphic profile, whereby the ratio of susceptibility alleles to resistance alleles provides an indication of whether the individual has or is at risk of Alzheimer's disease. Optionally, the polymorphic profile is determined at polymorphic sites in or within 10 kb of at least ten genes selected from the group, and presence of at least twenty susceptibility and resistance alleles is determined, and a ratio of resistance to susceptibility alleles of less than fifty percent is an indication that the individual does not have Alzheimer's disease. Optionally, the the polymorphic profile is determined in an individual having a symptom of, or known susceptibility to, Alzheimer's disease.

In some methods, the at least two haplotype blocks do not include APOE or APP. In some methods, the at least two haplotype blocks each comprise at least one gene selected from the group consisting of CACNA1C, CKM, FARS1, KIAA1486, LOC147468, LOC166522, MGC39715, NCE2, PCBP3, PDE11A, PFKFB2, SEC13L1, and TTLL2. In some methods, the polymorphic profile is determined in at least ten haplotype blocks, each including a different gene selected from the group. In some methods, the polymorphic profile is determined in at least two and no more than 50 different haplotype blocks. Some methods also involve selecting a treatment or prophylactic regime for an Alzheimer's related disease based on the polymorphic profile.

The invention further provides methods of diagnosing or prognosticating Alzheimer's disease in a subject. Such methods comprise determining a polymorphic profile in a haplotype block of a subject including gene selected from the group consisting of APOC1, CACNA1C, CKM, FARS1, KIAA1486, LOC147468, LOC166522, MGC39715, NCE2, PCBP3, PDE11A, PFKFB2, PVRL2, SEC13L1, TOMM40 and TTLL2.

The invention further provides methods of diagnosing or prognosticating late-onset Alzheimer's disease. Such methods comprise determining a polymorphic profile in a haplotype block that includes a gene selected from the group consisting of APP, APOC1, CACNA1C, CKM, FARS1, KIAA1486, LOC147468, LOC166522, MGC39715, NCE2, PCBP3, PDE11A, PFKFB2, PVRL2, SEC13L1, TOMM40 and TTLL2. Some methods determine presence of a susceptibility allele shown in Table 11 or in linkage disequilibrium therewith, the susceptibility allele indicating presence or susceptibility to the late-onset Alzheimer's disease.

The invention further provides methods of diagnosing or prognosticating an Alzheimer's related disease in a patient. Such methods determine presence of at least one susceptibility allele shown in Table 11 or in linkage disequilibrium therewith, the presence of the susceptibility allele indicating presence or susceptibility to the Alzheimer's related disease. Optionally, the method determines presence of at least one susceptibility allele shown in Table 11. Optionally, the method determines at least one susceptibility allele not in or within 40 kb of a gene selected from the group consisting of APP or APOE.

Any of the above methods can include informing the patient or a relative thereof of presence or susceptibility to an Alzheimer's related disease; or further comprising performing a secondary test for an Alzheimer's related disease, such as determining mental activity by a psychometric measure or taking a biopsy; administering a regime effective to treat or effect prophylaxis of an Alzheimer's related disease. Any of the above methods can also involve determining at least one susceptibility allele not in or within 40 kb of TOMM40 or APOC1, or not in or within 40 kb of LU, PVRL2, TOMM40, APOC1, APOC4, APOC2, or CLPTM1.

Optionally, any of the above methods determine presence of at least 5 or 10 susceptibility alleles in at least five different genes selected from the group consisting of APOE, APP, APOC1, CACNA1C, CKM, FARS1, KIAA1486, LOC147468, LOC166522, MGC39715, NCE2, PCBP3, PDE11A, PFKFB2, PVRL2, SEC13L1, TOMM40, and TTLL2, preferably susceptibility alleles are alleles shown Table 11.

The invention further provides methods of expression profiling. Such methods entail determining expression levels of at least 2 and no more than 10,000 genes in a subject, wherein at least two of the genes are selected from the group consisting of APOE, APP, APOC1, CACNA1C, CKM, FARS1, KIAA1486, LOC147468, LOC166522, MGC39715, NCE2, PCBP3, PDE11A, PFKFB2, PVRL2, SEC13L1, TOMM40, and TTLL2, the expression levels forming an expression profile. Optionally, the methods determine expression levels of the genes in a control subject free of an Alzheimer's related disease. Optionally the methods determine expression levels of the genes in a control subject having an Alzheimer's related disease. Optionally, the methods compare the expression levels of the genes in the subject with expression levels of the genes in a control subject known to have an Alzheimer's related disease and/or a control subject known to lack an Alzheimer's related disease, wherein similarity of expression profiles in the subject and the control subject having the Alzheimer's related disease is an indication the subject has the Alzheimer's related disease, and similarity of the expression profiles in the subject and the control subject not having the Alzheimer's related disease is an indication the subject lacks presence or susceptibility to the Alzheimer's related disease. In some methods, the the expression levels of at least two genes selected from the group consisting of APOC1, CACNA1C, CKM, FARS1, KIAA1486, LOC147468, LOC166522, MGC39715, NCE2, PCBP3, PDE11A, PFKFB2, PVRL2, SEC13L1, TOMM40, and TTLL2 are determined. In some methods, the determining step determines the expression level of at least 2 and no more than 100 genes, wherein the at least two genes are selected from the group consisting of APOC1, CACNA1C, CKM, FARS1, KIAA1486, LOC147468, LOC166522, MGC39715, NCE2, PCBP3, PDE11A, PFKFB2, PVRL2, SEC13L1, TOMM40, and TTLL2.

The invention further provides a transgenic non-human animal comprising a genome comprising a transgene comprising an exogenous nucleic acid encoding the protein of a gene selected from the group consisting of APOC1, CACNA1C, CKM, FARS1, KIAA1486, LOC147468, LOC166522, MGC39715, NCE2, PCBP3, PDE11A, PFKFB2, PVRL2, SEC 13L1, TOMM40, and TTLL2, whereby the animal expresses the gene, and is disposed to develop at least one sign or symptom of an Alzheimer's related disease.

The invention further provides a transgenic non-human animal comprising a genome comprising a transgene comprising an exogenous nucleic acid encoding the protein encoded by a gene selected from the group consisting of APP, wherein the exogenous gene has a susceptibility allele shown in Table 11 or in linkage disequilibrium therewith, whereby the animal expresses the gene, and is disposed to develop at least one sign or symptom of an Alzheimer's related disease. Optionally, the susceptibility allele is shown in Table 11.

The invention further provides a transgenic non-human animal comprising a genome having an enhanced, inhibited or disrupted endogenous gene that is the cognate form of a human gene selected from the group consisting of APOC1, CACNA1C, CKM, FARS1, KIAA1486, LOC147468, LOC166522, MGC39715, NCE2, PCBP3, PDE11A, PFKFB2, PVRL2, SEC13L1, TOMM40, and TTLL2, whereby the transgenic-nonhuman animal develops at least one sign or symptom of an Alzheimer's related disease.

The invention further provides a method for producing a transgenic knock-out non-human animal. The method entails providing a targeting construct containing a disrupted segment of a gene selected from the group consisting of APOC1, CACNA1C, CKM, FARS1, KIAA1486, LOC147468, LOC166522, MGC39715, NCE2, PCBP3, PDE11A, PFKFB2, PVRL2, SEC13L1, TOMM40, and TTLL2, and homologously recombining the targeting construct with the genome of a cell of the animal, whereby the construct is stably integrated into the genome of the cell; and propagating a transgenic animal from the cell.

The invention further provides a method for producing a transgenic non-human animal. The method entails introducing a construct encoding and capable of expressing the protein encoded by a gene selected from the group consisting of APOC1, CACNA1C, CKM, FARS1, KIAA1486, LOC147468, LOC166522, MGC39715, NCE2, PCBP3, PDE11A, PFKFB2, PVRL2, SEC13L1, TOMM40, and TTLL2, into a cell, and propagating a transgenic animal from the cell.

The invention further provides a method for identifying an agent for use in diagnosis, prognosis, prophylaxis, or treatment, of an Alzheimer's related disease. The method entails contacting a polypeptide encoded by a gene selected from the group consisting of APOC1, CACNA1C, CKM, FARS1, KIAA1486, LOC147468, LOC166522, MGC39715, NCE2, PCBP3, PDE11A, PFKFB2, PVRL2, SEC13L1, TOMM40, and TTLL2, or a nucleic acid encoding the polypeptide, with an agent to be tested; assessing a level of binding of the agent to the polypeptide or a level of modulation of activity or expression of the polypeptide by the agent; and comparing the level of binding activity or expression of the polypeptide with a control sample in an absence of the agent, wherein a difference in level of binding, activity or expression in the presence of the agent relative to the control sample is an indication that the agent has activity useful in diagnosis, prognosis, prophylaxis, or treatment, an Alzheimer's related disease. Optionally, the polypeptide is an isolated polypeptide. Optionally, the polypeptide is expressed in a cell transformed with a nucleic acid encoding the polypeptide. Optionally, the method also involves determining whether the agent shows activity inhibiting development of or clearing a sign or symptom of the Alzheimer's related disease in an animal model. Optionally the assessing involves contacting the agent with the polypeptide and detecting specific binding between the compound and the polypeptide or detecting a modulation of activity of the polypeptide or detecting a modulation of expression of the polypeptide.

The invention further provides methods of effecting treatment or prophylaxis of an Alzheimer's related disease. Such methods comprise administering to the subject an effective amount of an agent that modulates the activity or expression of a protein encoded by a gene selected from the group consisting of APOC1, CACNA1C, CKM, FARS1, KIAA1486, LOC147468, LOC166522, MGC39715, NCE2, PCBP3, PDE11A, PFKFB2, PVRL2, SEC13L1, TOMM40, and TTLL2. Optionally, the agent is selected from the group consisting of: an antibody, small molecule or natural product that specifically binds to a protein encoded by a gene selected from the group; a zinc finger protein that modulates expression of a gene selected from the group; an siRNA, antisense RNA, RNA complementary to a regulatory sequence, or ribozyme that inhibits expression of a gene selected from the group. Optionally, the method also involvese monitoring a sign or symptom of the Alzheimer's related disease in the patient responsive to the administration. Optionally, the method involves administering a second agent effective to effect treatment or prophylaxis of the Alzheimer's related disease. In some methods, the patient is human. In some methods, the disease is late-onset Alzheimer's disease.

The invention further provides a computer-implemented method of identifying a polymorphic profile characterizing a patient as amenable to treatment with an agent. Some method involve providing data for a first population of patients with an Alzheimer's related disease treated with the agent and a second population of patients with the disease treated with a placebo, the data comprising whether the patient reached a desired endpoint, and a polymorphic profile of the patients in the first and second populations in at least one polymorphic site in a gene selected from the group consisting of APOC1, CACNA1C, CKM, FARS1, KIAA1486, LOC147468, LOC166522, MGC39715, NCE2, PCBP3, PDE11A, PFKFB2, PVRL2, SEC13L1, TOMM40, and TTLL2, and selecting first and second subpopulations from the first and second populations based on similarity of the polymorphic profile; and comparing the percentage of patients in the first subpopulation reaching the desired endpoint with the percentage of patients in the second subpopulation, a significant different indicating that the polymorphic profile of the subpopulations characterizes a patient as amenable to treatment.

The invention further provides an isolated protein encoded by LOC147468 in which SNP at SSID 24218353 is occupied by the nucleotide A.

The invention further provides an antibody that specifically binds to an isolated protein encoded by LOC147468 in which SNP at SSID 24218353 is occupied by the nucleotide A but not a G or vice versa.

The invention further provides a method of screening an agent for activity in treating an Alzheimer's related disease comprising performing a primary screen to determine whether the agent affects level of expression or function of a protein encoded by a gene selected from the group consisting of APOE, APP, APOC1, CACNA1C, CKM, FARS1, KIAA1486, LOC147468, LOC166522, MGC39715, NCE2, PCBP3, PDE11A, PFKFB2, PVRL2, SEC13L1, TOMM40, and TTLL2, and performing a secondary screen to determine whether the agent affects the Alzheimer's related disease in an animal. Optionally, the primary screen measures binding of the agent to the protein. Optionally, the primary screen measures capacity of the agent to agonize or antagonize the protein.

The invention further provides a method for identifying a polymorphic site correlated with Alzheimer's disease or susceptibility thereto, comprising identifying a polymorphic site within a protein encoded by a gene selected from the group consisting APP, APOC1, CACNA1C, CKM, FARS1, KIAA1486, LOC147468, LOC166522, MGC39715, NCE2, PCBP3, PDE11A, PFKFB2, PVRL2, SEC13L1, TOMM40, and TTLL2, and determining whether a variant polymorphic form occupying the site is associated with the disease or susceptibility thereto, provided that if the gene is APP, the disease is late-onset Alzheimer's disease.

The invention further provides a method of excluding an individual from a clinical trial to test a drug for treatment or prophylaxis of Alzheimer's disease. Such a method entails determining a polymorphic profile in an individual presenting symptoms resembling Alzheimer's disease in or within 10 kb of a plurality of genes selected from the group consisting of APOE, APP, APOC1, CACNA1C, CKM, FARS1, KIAA1486, LOC147468, LOC166522, MGC39715, NCE2, PCBP3, PDE11A, PFKFB2, PVRL2, SEC13L1, TOMM40 and TTLL2; determining the total number of resistance and susceptibility alleles at each locus in the polymorphic profile, wherein a high ratio of resistance to susceptibility alleles is an indication the individual should be excluded from the clinical trial.

The invention further provides a method of polymorphic profiling an individual. Such a method entails determining a polymorphic profile in at least two but no more than 1000 different haplotype blocks, at least two of the haplotype blocks including a gene selected from the group consisting of APOE, APP, APOC1, C9orf52, CTNND2, CUGBP1, DKFZP566K1924, FARS1, FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC388110, MGC39715, NCE2, PFKFB2, PPP1R12B, PSEN1, TGD5, and TTLL2. In some methods, the polymorphic profile in the at least two haplotype blocks is determined in or within 10 kb of the selected genes. In some methods, the polymorphic profile is determined in an individual having a symptom of, or known susceptibility to, Alzheimer's disease. In some methods, the at least two haplotype blocks do not include APOE, APP and PSEN1. In some methods, the at least two haplotype blocks each comprise at least one gene selected from the group consisting of A2BP1, C9orf52, CTNND2, CUGBP1, DKFZP566K1924, FARS1, FGL2, FLJ14442, FLJ36760, KIAA1486, KIAA1862, LNX2,LOC147468, LOC283867, LOC401237, LRP1B, MATN3, MRLC2, PCBP3, PDE11A, PVRL2, SEC13L1, SOX5 and TOMM40. Some methods determine the polymorphic profile in at least ten haplotype blocks, each including a gene selected from the group consisting of A2BP1, C9orf52, CTNND2, CUGBP1, DKFZP566K1924, FARS1, FGL2, FLJ14442, FLJ36760, KIAA1486, KIAA1862, LNX2,LOC147468, LOC283867, LOC401237, LRP1B, MATN3, MRLC2, PCBP3, PDE11A, PVRL2, SEC13L1, SOX5 and TOMM40. Optionally, the polymorphic profile is determined in at least two and no more than 50 different haplotype blocks. Some methods also involve comprise selecting a treatment or prophylactic regime for an Alzheimer's related disease based on the polymorphic profile.

The invention further provides a method of diagnosing or prognosticating Alzheimer's disease. The method involves determining a polymorphic profile in a haplotype block including gene selected from the group consisting of C9orf52, CTNND2, CUGBP1, DKFZP566K1924, FARS1, FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC388110, MGC39715, NCE2, PFKFB2, PPP1R12B, TGD5, and TTLL2.

The invention further provides a method of diagnosing or prognosticating late-onset Alzheimer's disease. The method involves determining a polymorphic profile in a haplotype block that includes a gene selected from the group consisting of APP, PSEN1, C9orf52, CTNND2, CUGBP1, DKFZP566K1924, FARS1, FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC388110, MGC39715, NCE2, PFKFB2, PPP1R12B, TGD5, and TTLL2. Optionally the determining determines presence of a susceptibility allele at a SNP selected from SNP ID 42182, 42256, 42263 and 201402 or a SNP in linkage disequilibrium with any of these SNP IDs; the presence of the susceptibility allele indicating presence or susceptibility to the Alzheimer's related disease.

The invention further provides a method of diagnosing or prognosticating an Alzheimer's related disease in a patient. The method involves determining presence of at least one susceptibility allele at a single nucleotide polymorphism position shown in column 9 of FIG. 3 or in linkage disequilibrium therewith, the presence of the susceptibility allele indicating presence or susceptibility to the Alzheimer's related disease. Optionally, the determining determines at least one susceptibility allele not in or within 40 kb of a gene selected from the group consisting of APP, PSEN1 or APOE.

Any of the above methods can involve informing the patient or a relative thereof of presence or susceptibility to an Alzheimer's related disease, or performing a secondary test for an Alzheimer's related disease, such as determining mental activity by a psychometric measure or taking a biopsy, or administering a regime effective to treat or effect prophylaxis of an Alzheimer's related disease.

Any of the method can involve determining determines at least one susceptibility allele not in or within 40 kb of TOMM40 or APOC1, or not in or within 40 kb of LU, PVRL2, TOMM40, APOC1, APOC4, APOC2, or CLPTM1.

Optionally any of the methods determines presence of at least 5 susceptibility alleles at single nucleotide polymorphic sites at a position shown in column 9 of FIG. 3, or in linkage disequilibrium therewith. Optionally, the determining determines presence of at least 5 susceptibility alleles in at least five different genes selected from the group consisting of A2BP1, APOE, APOC, APP, C9orf52, CTNND2, CUGBP1, DKFZP566K1924, FARS1, FGL2, FLJ14442, FLJ36760, KIAA1486, KIAA1862, LNX2,LOC147468, LOC283867, LOC401237, LRP1B, MATN3, MRLC2, PCBP3, PDE11A, PVRL2, SEC13L1, SOX5 and TOMM40. Optionally, the determining determines presence of at least 10 susceptibility alleles in at least ten different genes selected from the group consisting of A2BP 1, APOE, APOC, APP, C9orf52, CTNND2, CUGBP1, DKFZP566K1924, FARS1, FGL2, FLJ14442, FLJ36760, KIAA1486, KIAA1862, LNX2,LOC147468, LOC283867, LOC401237, LRP1B, MATN3, MRLC2, PCBP3, PDE11A, PVRL2, SEC13L1, SOX5 and TOMM40.

The invention further provides a method of expression profiling. The method involves determining expression levels of at least 2 and no more than 10,000 genes in a subject, wherein at least two of the genes are shown in FIG. 3, the expression levels forming an expression profile. Optionally, the method also involves determining expression levels of the genes in a control subject free of an Alzheimer's related disease. Optionally, the method also involves determining expression levels of the genes in a control subject having an Alzheimer's related disease. Optionally, the method also involves comparing the expression levels of the genes in the subject with expression levels of the genes in a control subject known to have an Alzheimer's related disease and/or a control subject known to lack an Alzheimer's related disease, wherein similarity of expression profiles in the subject and the control subject having the Alzheimer's related disease is an indication the subject has the Alzheimer's related disease, and similarity of the expression profiles in the subject and the control subject not having the Alzheimer's related disease is an indication the subject lacks presence or susceptibility to the Alzheimer's related disease. Optionally, the expression levels of at least two genes selected from the group consisting of APOE, APOC, APP, C9orf52, CTNND2, CUGBP1, DKFZP566K1924, FARS1,FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC3881 10, MGC39715, NCE2, PFKFB2, PPP1R12B, PSEN1, TGD5, and TTLL2 are determined. Optionally, the determining step determines the expression level of at least 2 and no more than 100 genes, wherein the at least two genes are selected from the group consisting of A2BP1, C9orf52, CTNND2, CUGBP1, DKFZP566K1924, FARS1, FGL2, FLJ14442, FLJ36760, KIAA1486, KIAA1862, LNX2,LOC147468, LOC283867, LOC401237, LRP1B, MATN3, MRLC2, PCBP3, PDE11A, PVRL2, SEC13L1, SOX5 and TOMM40.

The invention further provides a transgenic non-human animal comprising a genome comprising a transgene comprising an exogenous nucleic acid encoding the protein of a gene selected from the group consisting of APOC, A2BP 1, C9orf52, CTNND2, CUGBP1, DKFZP566K1924, FARS1, FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC388110, MGC39715, NCE2, PFKFB2, PPP1R12B, TGD5, and TTLL2, whereby the animal expresses the gene, and is disposed to develop at least one sign or symptom of an Alzheimer's related disease. Optionally, the gene is selected from the group consisting of A2BP1, C9orf52, CTNND2, CUGBP1, DKFZP566K1924, FARS1, FGL2, FLJ14442, FLJ36760, KIAA1486, KIAA1862, LNX2,LOC147468, LOC283867, LOC401237, LRP1B, MATN3, MRLC2, PCBP3, PDE11A, PVRL2, SEC13L1, SOX5 and TOMM40.

The invention further provides a transgenic non-human animal comprising a genome comprising a transgene comprising an exogenous nucleic acid encoding the protein encoded by a gene selected from the group consisting of APP and PSEN1, wherein the exogenous gene has a variant form associated with late-onset Alzheimer's disease, whereby the animal expresses the gene, and is disposed to develop at least one sign or symptom of an Alzheimer's related disease. Optionally, the susceptibility allele is at an SNP selected from SNP ID 42182, 42256, 42263 and 201402 in FIG. 3.

The invention further provides a transgenic non-human animal comprising a genome having an enhanced, inhibited or disrupted endogenous gene that is the cognate form of a human gene selected from the group consisting of APOC, C9orf52, CTNND2, CUGBP1, DKFZP566K1924, FARS1, FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC388110, MGC39715, NCE2, PFKFB2, PPP1R12B, TGD5, and TTLL2, whereby the transgenic-nonhuman animal develops at least one sign or symptom of an Alzheimer's related disease. Optionally, the gene is selected from the group consisting of A2BP1, C9orf52, CTNND2, CUGBP1, DKFZP566K1924, FARS1, FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5 and TOMM40.

The invention further provides a method for producing a transgenic knock-out non-human animal. The method involves providing a targeting construct containing a disrupted segment of a gene selected from the group consisting of APOC, C9orf52, CTNND2, CUGBP1, DKFZP566K1924, FARS1,FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC388110, MGC39715, NCE2, PFKFB2, PPP1R12B, TGD5, and TTLL2; and homologously recombining the targeting construct with the genome of a cell of the animal, whereby the construct is stably integrated into the genome of the cell; and propagating a transgenic animal from the cell.

The invention further provides a method for producing a transgenic non-human animal. The method entails introducing a construct encoding and capable of expressing the protein encoded by a gene selected from the group consisting of APOC, C9orf52, CTNND2, CUGBP1, DKFZP566K1924, FARS1, FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC388110, MGC39715, NCE2, PFKFB2, PPP1R12B, TGD5, and TTLL2into a cell, and propagating a transgenic animal from the cell.

The invention further provides a method for identifying an agent for use in diagnosis, prognosis, prophylaxis, or treatment, of an Alzheimer's related disease. The method entails contacting a polypeptide encoded by a gene selected from the group consisting of APOC, C9orf52, CTNND2, CUGBP 1, DKFZP566K1924, FARS1,FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC388110, MGC39715, NCE2, PFKFB2, PPP1R12B, TGD5, and TTLL2, or a nucleic acid encoding the polypeptide, with an agent to be tested; assessing a level of binding of the agent to the polypeptide or a level of modulation of activity or expression of the polypeptide by the agent; and comparing the level of binding activity or expression of the polypeptide with a control sample in an absence of the agent, wherein a difference in level of binding, activity or expression in the presence of the agent relative to the control sample is an indication that the agent has activity useful in diagnosis, prognosis, prophylaxis, or treatment, an Alzheimer's related disease. Optionally, the polypeptide is an isolated polypeptide. Optionally, the polypeptide is expressed in a cell transformed with a nucleic acid encoding the polypeptide. Optionally, the method also involves determining whether the agent shows activity inhibiting development of or clearing a sign or symptom of the Alzheimer's related disease in an animal model. Optionally, the assessing comprises contacting the agent with the polypeptide and detecting specific binding between the compound and the polypeptide. Optionally, the assessing comprises detecting a modulation of activity of the polypeptide. Optionally, the assessing comprises detecting a modulation of expression of the polypeptide.

The invention further provides a method of effecting treatment or prophylaxis of an Alzheimer's related disease. The method involves administering to the subject an effective amount of an agent that modulates the activity or expression of a protein encoded by a gene selected from the group consisting of APOC, C9orf52, CTNND2, CUGBP1, DKFZP566K1924, FARS1, FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC388110, MGC39715, NCE2, PFKFB2, PPP1R12B, PSEN1, TGD5, and TTLL2. Optionally, the agent is selected from the group consisting of: an antibody, small molecule or natural product that specifically binds to a protein encoded by a gene selected from the group; a zinc finger protein that modulates expression of a gene selected from the group; an siRNA, antisense RNA, RNA complementary to a regulatory sequence, or ribozyme that inhibits expression of a gene selected from the group. Optionally, the method also involves monitoring a sign or symptom of the Alzheimer's related disease in the patient responsive to the administration. Optionally, the method involves administering a second agent effective to effect treatment or prophylaxis of the Alzheimer's related disease. Optionally, the patient is a human. Optionally, the Alzheimer's related disease is late-onset Alzheimer's disease. Optionally, the gene is selected from the group consisting of A2BP 1, C9orf52, CTNND2, CUGBP1, DKFZP566K1924, FARS1, FGL2, FLJ14442, FLJ36760, KIAA1486, KIAA1862, LNX2,LOC147468, LOC283867, LOC401237, LRP1B, MATN3, MRLC2, PCBP3, PDE11A, PVRL2, SEC13L1, SOX5 and TOMM40

The invention further provides a computer-implemented method of identifying a polymorphic profile characterizing a patient as amenable to treatment with an agent. Such methods entail providing data for a first population of patients with an Alzheimer's related disease treated with the agent and a second population of patients with the disease treated with a placebo, the data comprising whether the patient reached a desired endpoint, and a polymorphic profile of the patients in the first and second populations in at least one polymorphic site in a gene selected from the group consisting of APOC, APOE, APP, C9orf52, CTNND2, CUGBP1, DKFZP566K1924, FARS1, FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC388110, MGC39715, NCE2, PFKFB2, PPP1R12B, PSEN1, TGD5, and TTLL2; selecting first and second subpopulations from the first and second populations based on similarity of the polymorphic profile; comparing the percentage of patients in the first subpopulation reaching the desired endpoint with the percentage of patients in the second subpopulation, a significant different indicating that the polymorphic profile of the subpopulations characterizes a patient as amenable to treatment.

The invention further provides for usse of (a) an isolated nucleic acid that specifically hybridizes to a segment in the human genome that includes a single nucleotide polymorphism (SNP) at a position shown in column 9 of FIG. 3 or is in linkage disequilibrium therewith, (b) a SNP shown in column 9 of FIG. 3 or in linkage disequilibrium therewith, or (c) a protein encoded by the nucleic acid, or (d) an antibody that specifically binds to the protein for diagnosis, prognosis, prophylaxis, treatment or study of an Alzheimer's related disease. In some uses, the segment is located no further than 10 kb from the SNP. In some uses, the segment is within a gene including the SNP or in linkage equilibrium therewith. Optionally, the isolated nucleic acid is a probe or primer. Optionally, the isolated nucleic acid is a cDNA. Optionally, the isolated nucleic acid is a gene shown in FIG. 3. Optionally, the segment is not within the human ApoE1 gene or in linkage disequilibrium therewith. If the segment is in an amyloid precursor protein gene or a presenilin-1 gene, then optionally the Alzheimer's related disease is not early onset Alzheimer's disease. Optionally, the disease is late-onset Alzheimer's disease. Optionally, the segment is within a gene selected from the group consisting of amyloid precursor protein, presenilin-1, AS2BP1, C9orf52, CTNND2, CUGBP1, DKFZP566, FARS1, FGL2, FLj14442, FLJ36760, KIAA1486, KIAA1862, LNX2, LOC147468, LOC283867, LOC401237, LRP1B, MATN#, MRLC2, PCBP3, PDE11A, PVRL2, SEC13L1, SOX5, and TOMM40.

The invention further provides an isolated protein encoded by a gene shown in FIG. 3, wherein at least one amino acid of the gene is encoded by a codon that includes a variant form of a polymorphic site shown in column 5 of FIG. 3. Optionally, the gene is selected from the group consisting of fibrinogen-like 2, FLJ36760, hypothetical protein FLJ38736.

The invention further provides an antibody that specifically binds to a protein encoded by a gene selected from the group consisting of APOE, APP, APOC1, C9orf52, CTNND2, CUGBP1, DKFZP566K1924, FARS1, FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC388110, MGC39715, NCE2, PFKFB2, PPP1R12B, PSEN1, TGD5, and TTLL2 in which an amino acid of the protein is encoded by a nucleic acid in which an SNP shown in FIG. 3 is occupied by the nucleotide of allele 1 and not the nucleotide of allele 2, vice versa.

The invention further provides a method of screening an agent for activity in treating an Alzheimer's related disease comprising performing a primary screen to determine whether the agent affects level of expression or function of a protein encoded by a gene in FIG. 3 other than APP, APOE and PSEN1, and performing a secondary screen to determine whether the agent affects the Alzheimer's related disease in an animal. Optionally, the primary screen measures binding of the agent to the protein. Optionally, the primary screen measures capacity of the agent to agonize or antagonize the protein.

The invention further provides a method of screening an agent for activity in treating an Alzheimer's related disease comprising exposing a transgenic animal as defined in any of claims 31-35 to the agent; and determining whether the agent treats or inhibits further development of the disease in the animal model.

The invention further provides a method for identifying a polymorphic site correlated with Alzheimer's disease or susceptibility thereto, comprising identifying a polymorphic site within a protein encoded by a gene in FIG. 3, and determining whether a variant polymorphic form occupying the site is associated with the disease or susceptibility thereto.

### BRIEF DESCRIPTION OF THE DRAWINGS

FIG. 1 illustrates variants in the APOE region that are related to resistance or susceptibility to AD.

FIG. 2 illustrates variants from various genomic regions that are related to resistance or susceptibility to AD.

FIG. 3 illustrates information concerning SNPs of interest.

FIG. 4 shows that when the samples were tested for two distinct populations, the cases and controls showed similar distributions of inferred population memberships.

FIG. 5 shows the distribution of genomic control-corrected trend test p-values for the SNPs selected from throughout the genome by pooled genotyping (a), and the SNPs from the Chromosome 10 (b), 12 (c), and 19 (d) candidate regions.

### DEFINITIONS

The term “a” or “an” as used herein may mean one or more.

The term “Alzheimer's Disease” or “AD” is defined broadly to include asymptomatic as well as symptomatic conditions of Alzheimer's disease including genetic predisposition for AD, environmentally induced AD, early-onset AD, late-onset AD (LOAD), familial AD (FAD).

The term “AD-related disease” refers to one or more diseases, conditions or symptoms or susceptibility to diseases, conditions or symptoms that involve directly or indirectly, neurodegeneration including but not limited to the following: Alzheimer's disease (AD), amyotrophic lateral sclerosis (ALS), Alpers' disease, Batten disease, Cockayne syndrome, corticobasal ganglionic degeneration, Huntington's disease, Lewy body disease, Pick's disease, motor neuron disease, multiple system atrophy, olivopontocerebellar atrophy, Parkinson's disease, postpoliomyelitis syndrome, prion diseases, progressive supranuclear palsy, Rett syndrome, Shy-Drager syndrome and tuberous sclerosis, and may be characterized by, e.g., dementia, memory loss, confusion, and other neurodegenerative conditions. In certain aspects, an AD-related disease is a neurodegenerative disease the affects neurons in the brain. An AD-related disease may be e.g. a condition that is a risk factor for developing AD, or may be a condition for which AD is a risk factor, or both. Many Alzheimer's related diseases are characterized by related pathology of amyloid deposits of a protein that stain with Congo red dye and/or related neurodegenerative symptoms.

Susceptibility to AD or a related disease means that a subject has a significantly greater risk of developing the disease than the average risk of an age-, and sex-matched individual from the general population.

Resistance to AD or related disease means that a subject has a significantly lower risk of developing the disease than the average risk of an age- and sex-matched individual from the general population.

A nucleic acid or polypeptide associated with susceptibility (e.g., a susceptibility allele) to a disease is a nucleic acid or polypeptide that occurs significantly more frequently in a population of individuals having the disease than in a population of individuals lacking the disease.

A nucleic acid or polypeptide associated with resistance to a disease (e.g., a resistance allele) is a nucleic acid or polypeptide that occurs significantly less frequently in a population of individual having the disease than in a population of individuals lacking the disease.

A symptom of a disorder means a phenomenon experienced by an individual having the disorder indicating a departure from normal function, sensation or appearance.

A sign of a disorder is any bodily manifestation that serves to indicate presence or risk of a disorder.

The term “AD nucleic acid” or “AD associated genomic region” means a nucleic acid, or fragment, derivative, variant, polymorphism, or complement thereof, associated with resistance or susceptibility to AD-related disease including, for example, at least one or more AD polymorphisms, genomic regions spanning 10 kb immediately upstream and 10 kb immediately downstream of an AD polymorphism, coding and non-coding regions of an associated gene, and/or genomic regions spanning 10 kb immediately upstream and 10 kb immediately downstream of an associated gene, and variants thereof. The term also includes nucleic acids similarly related to genes in an associated gene pathway. An AD nucleic acid may also be an “associated genomic region” when it is found within the genome of an organism.

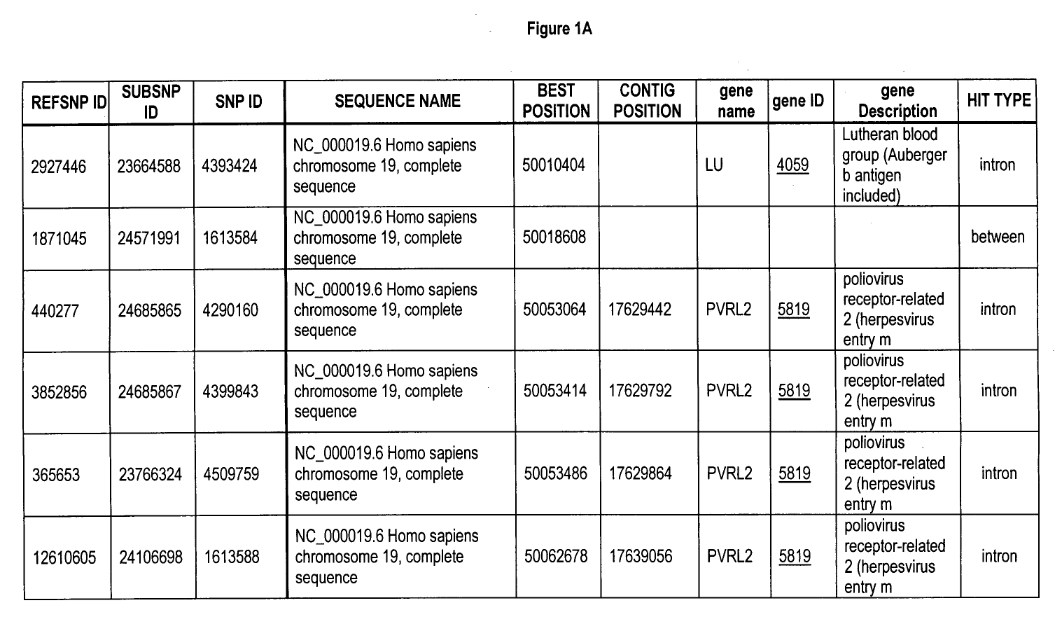

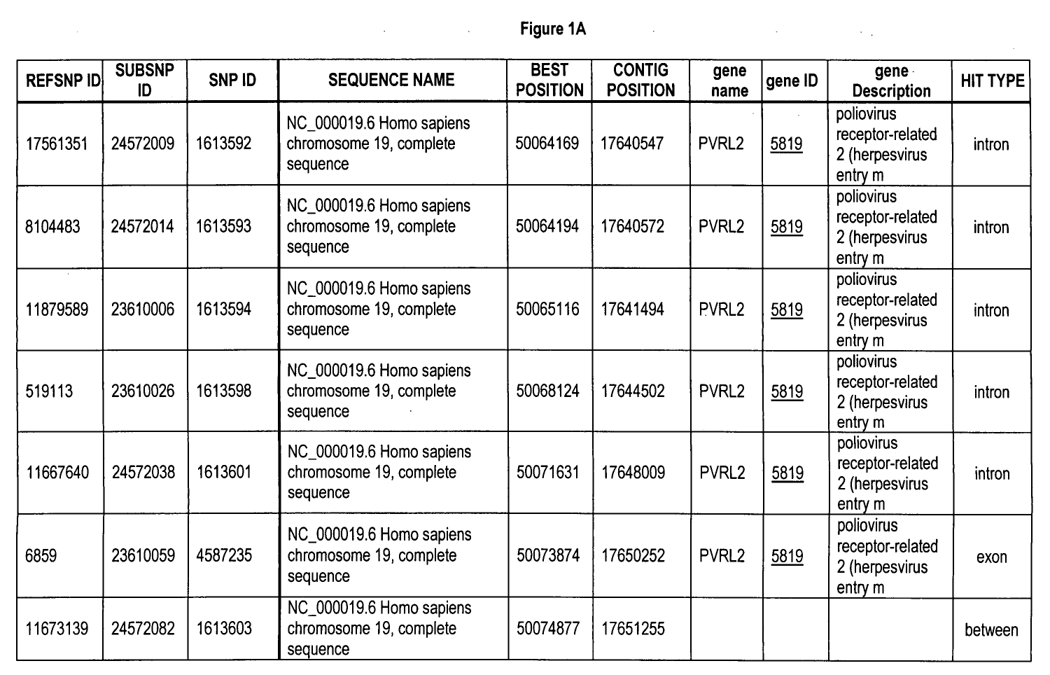

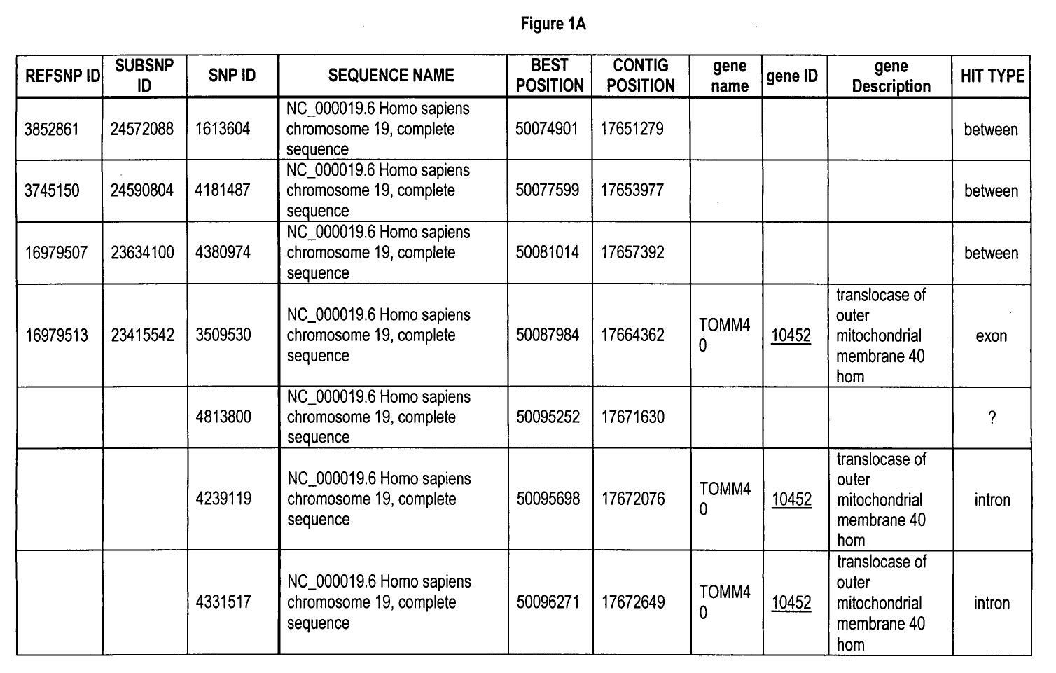

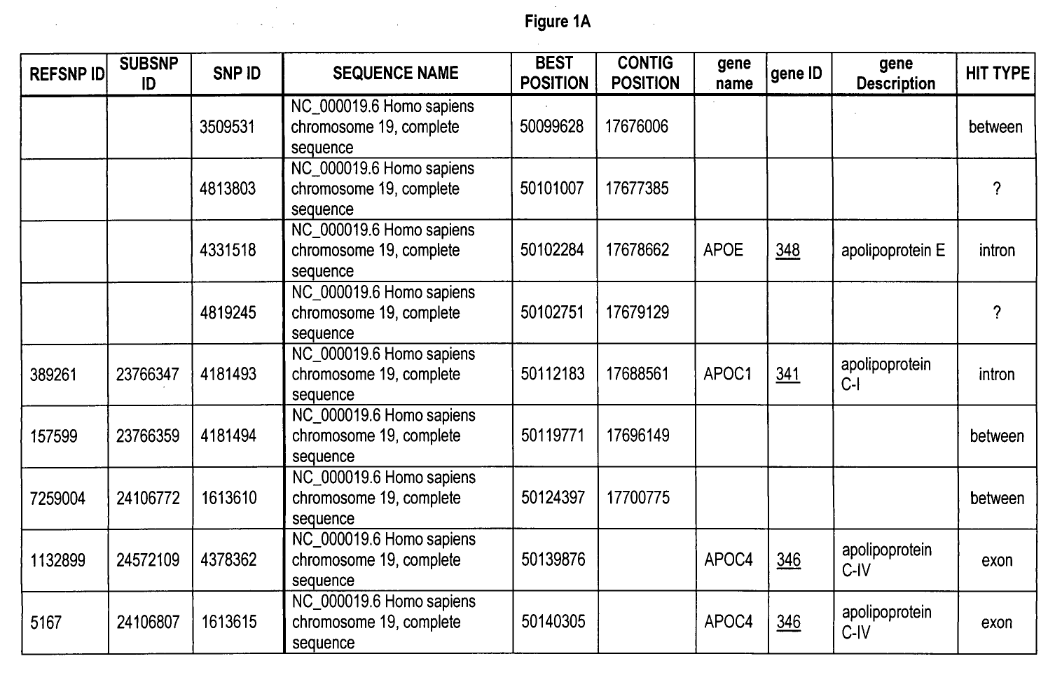

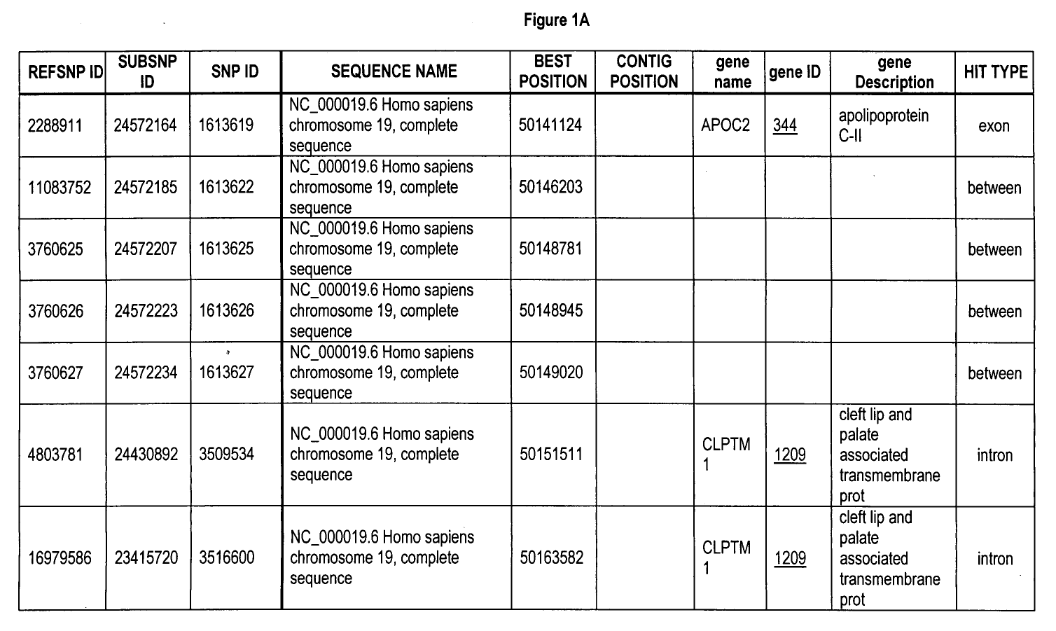

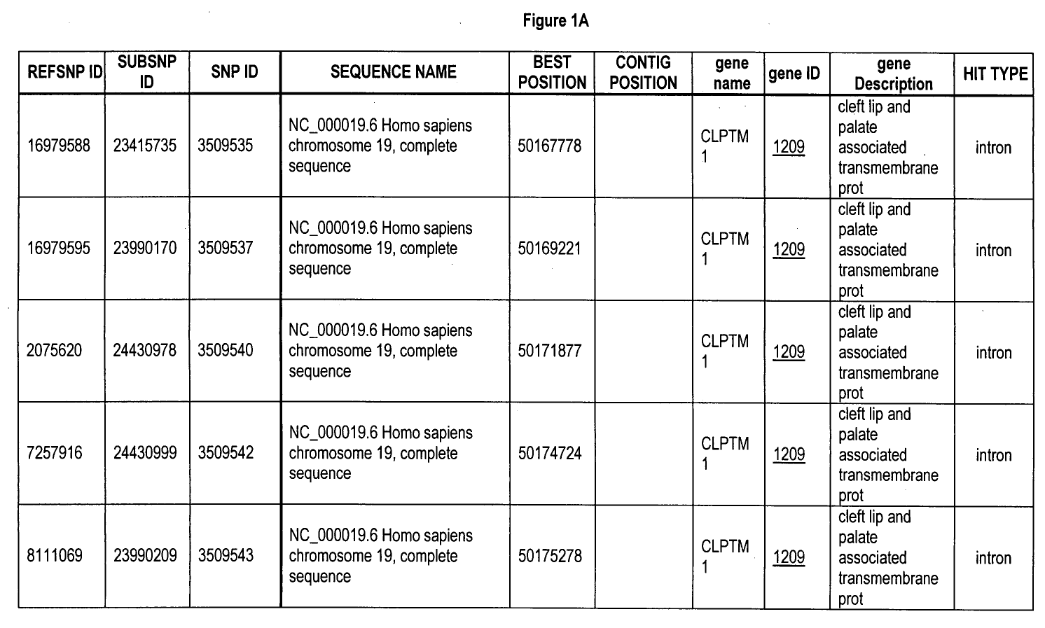

The term “AD polymorphism” or “associated polymorphism” refers to a specific nucleic acid locus at which a nucleotide polymorphism associated with AD-related disease occurs. For example, an AD polymorphism may be a SNP position such as those provided in FIGS. 1, 2 and 3. There may be two or more nucleotide base variants (“alleles”) at a given AD polymorphism, and each of these alleles may be specifically associated with either a resistance or a susceptibility to AD-related disease, or to a response to a treatment regimen (e.g., drug response). An allele that is the same as that found in a reference nucleic acid sequence is referred to as a “reference allele”, and an allele that is different than that found in the reference sequence is referred to as an “alternate allele”.

The term “AD polypeptide” or “associated polypeptide” refers to any peptide, polypeptide, or fragment, derivative or variant thereof, associated with resistance or susceptibility to AD-related disease, including a peptide or polypeptide regulated or encoded, in whole or in part, by an associated gene or genomic regions of 10 kb immediately upstream and downstream of an associated gene, or fragment, variants, derivative, or modifications thereof. The term also includes such polypeptides up- or down-stream in an associated gene pathway.

The term “another” as used herein may mean at least a second or more.

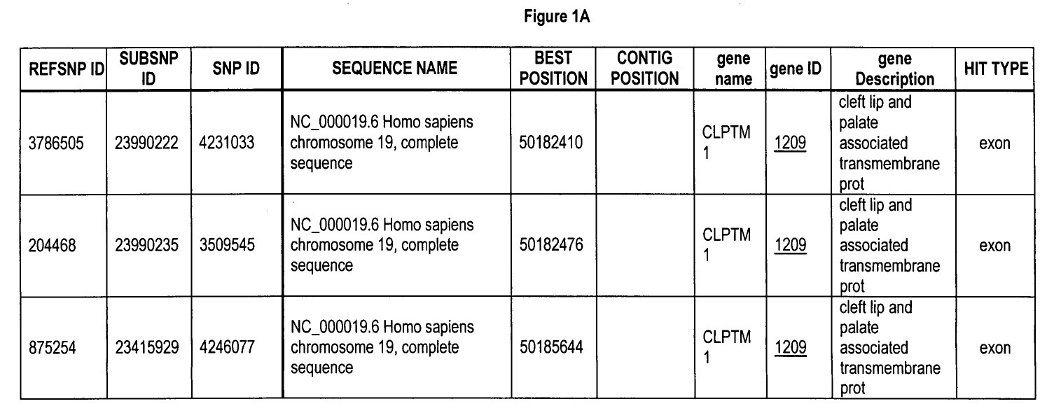

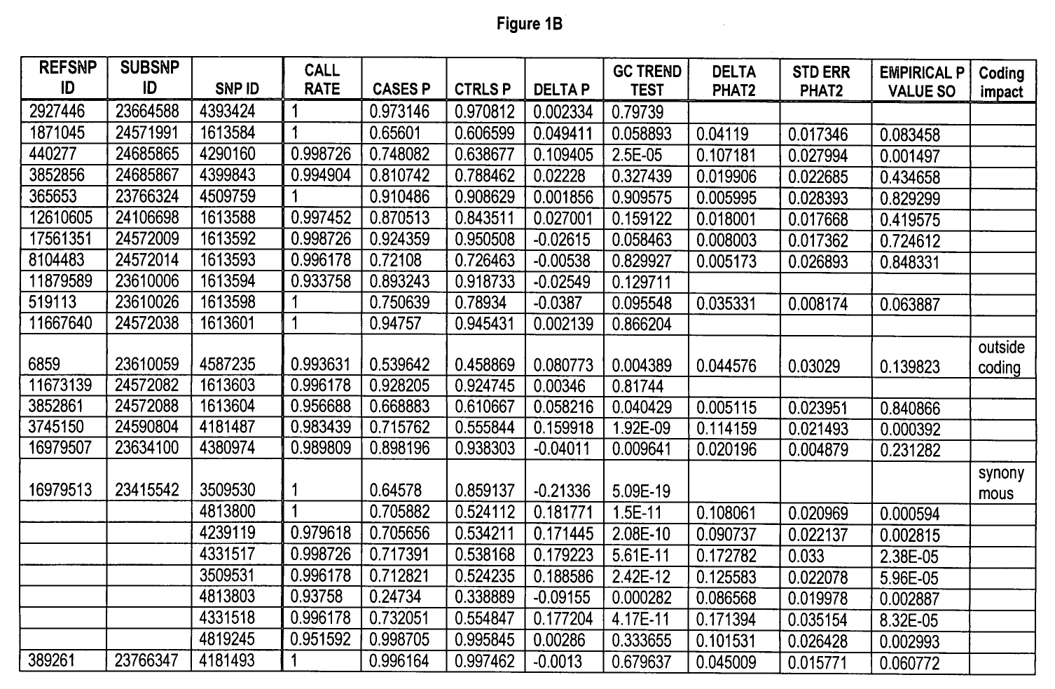

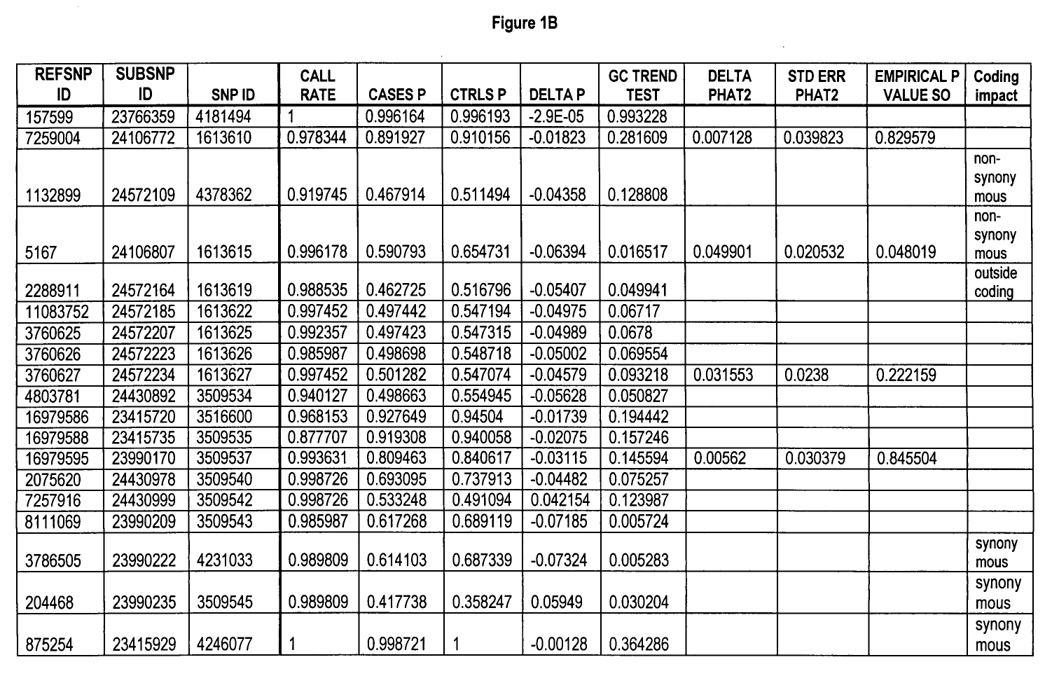

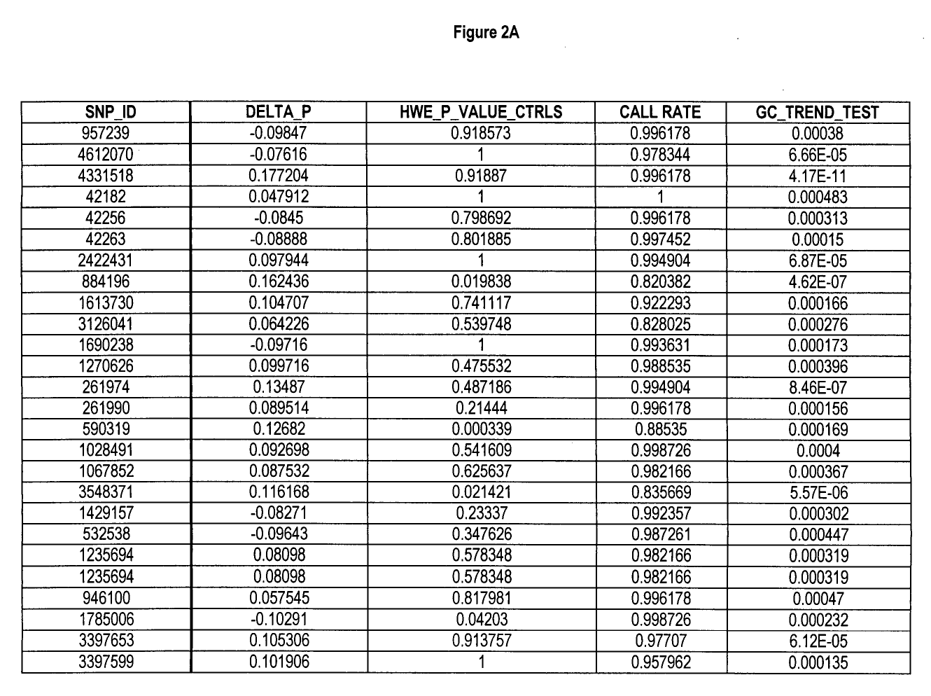

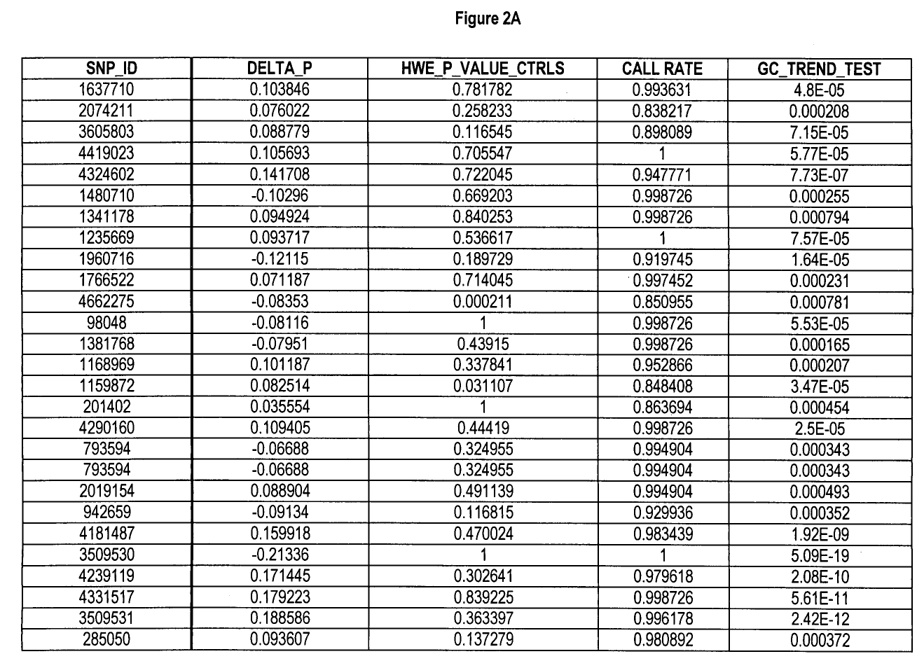

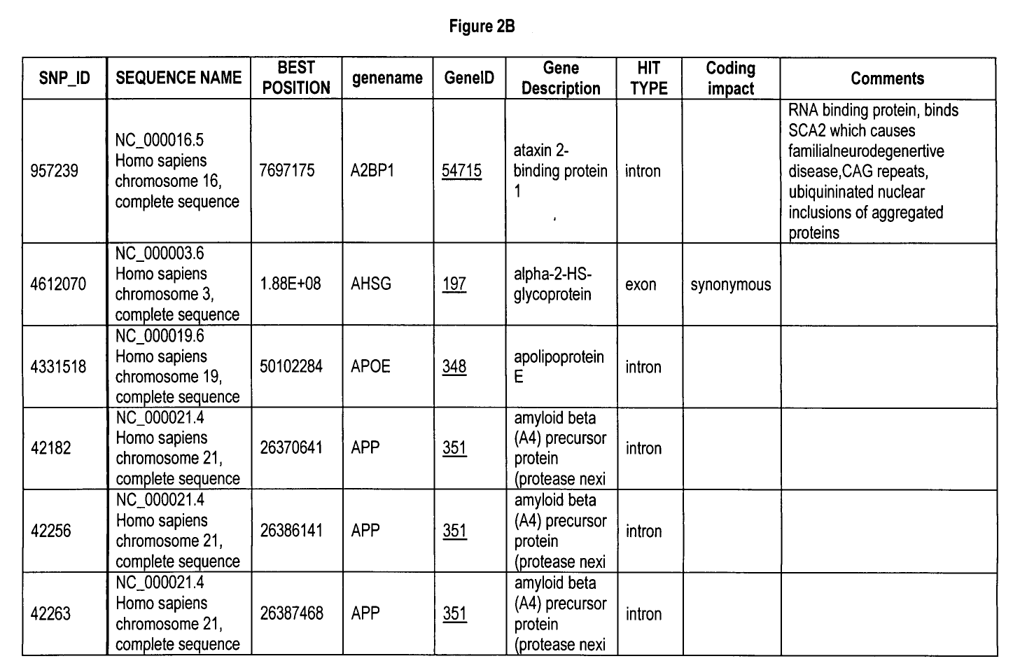

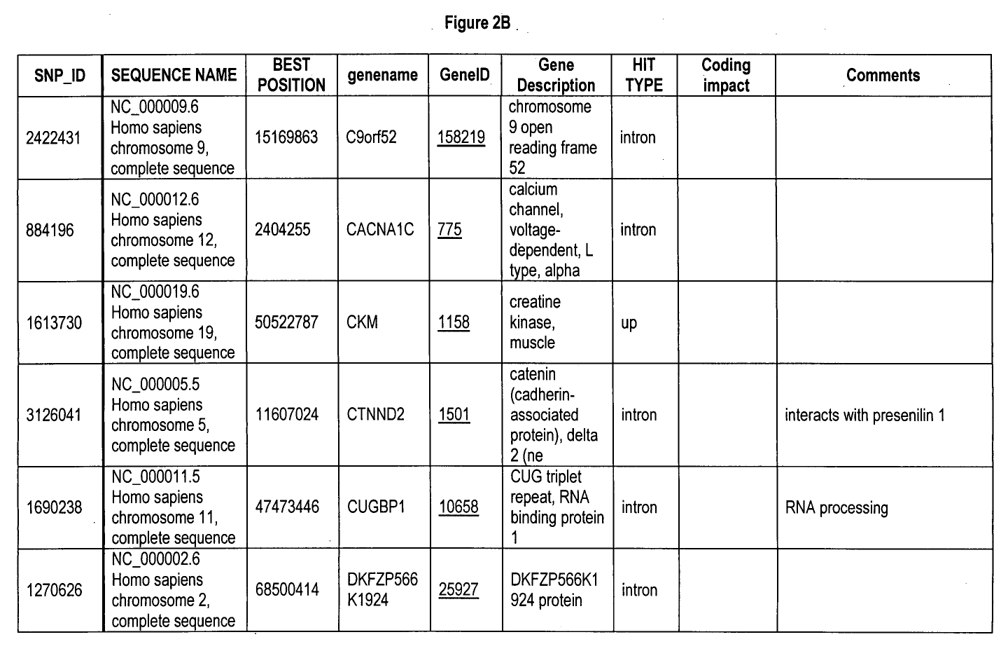

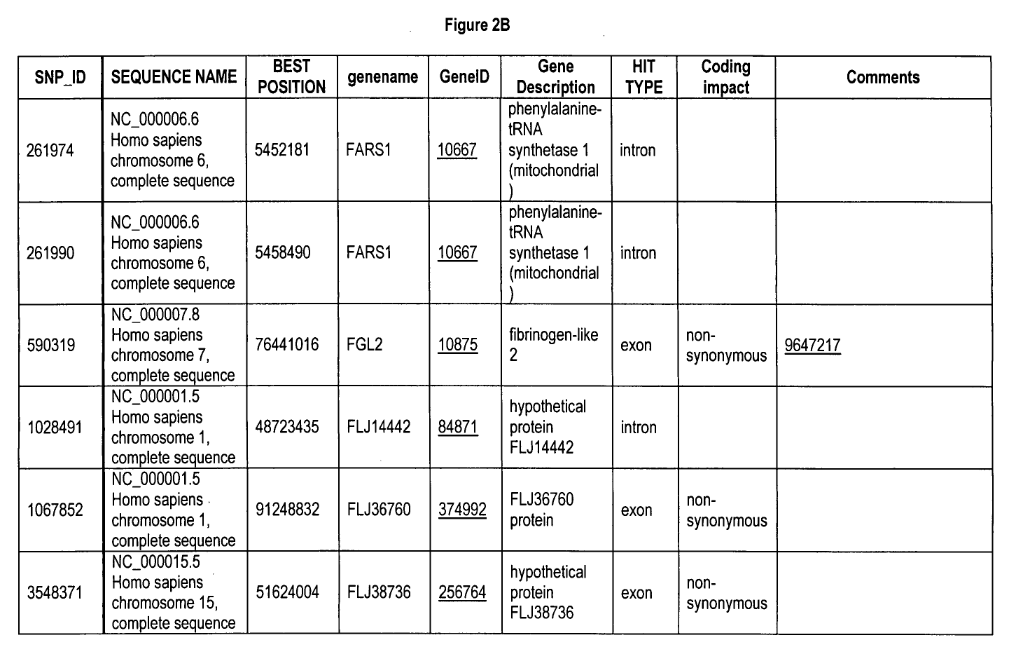

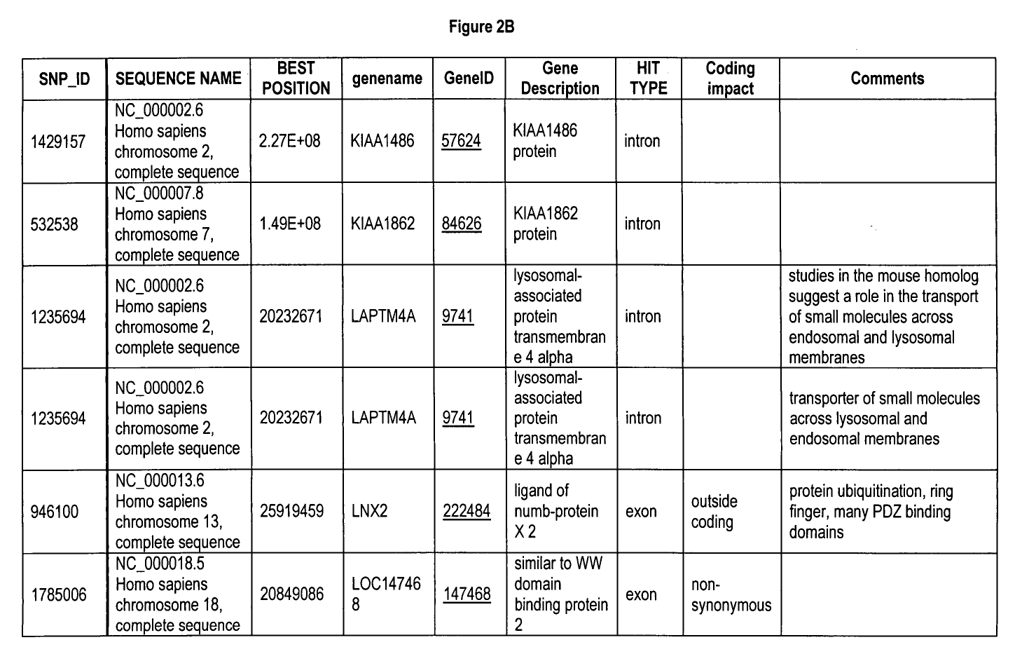

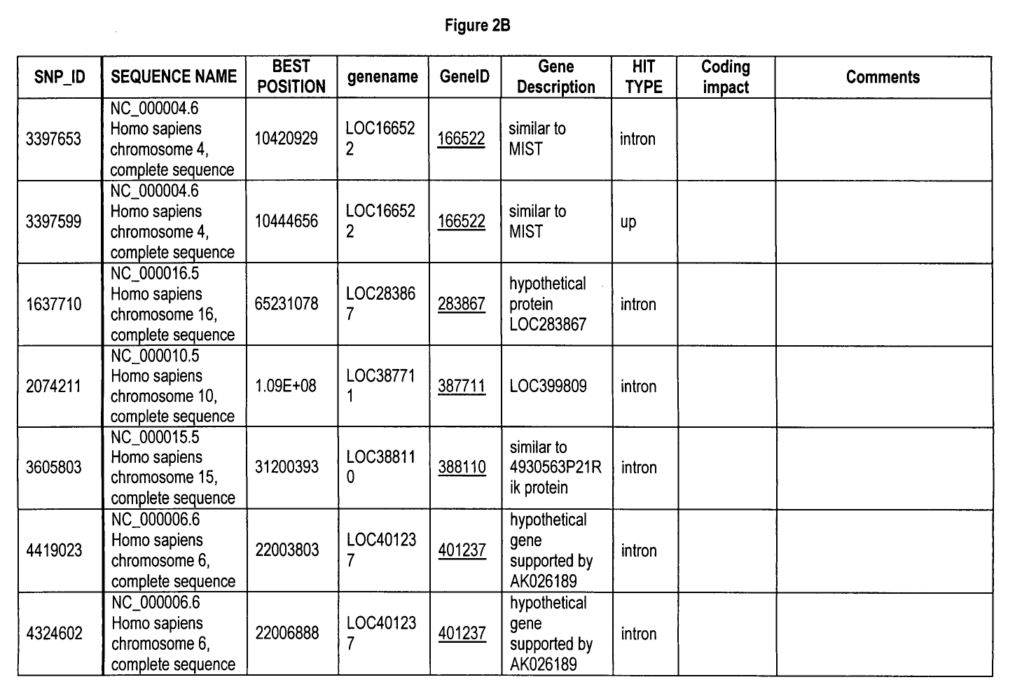

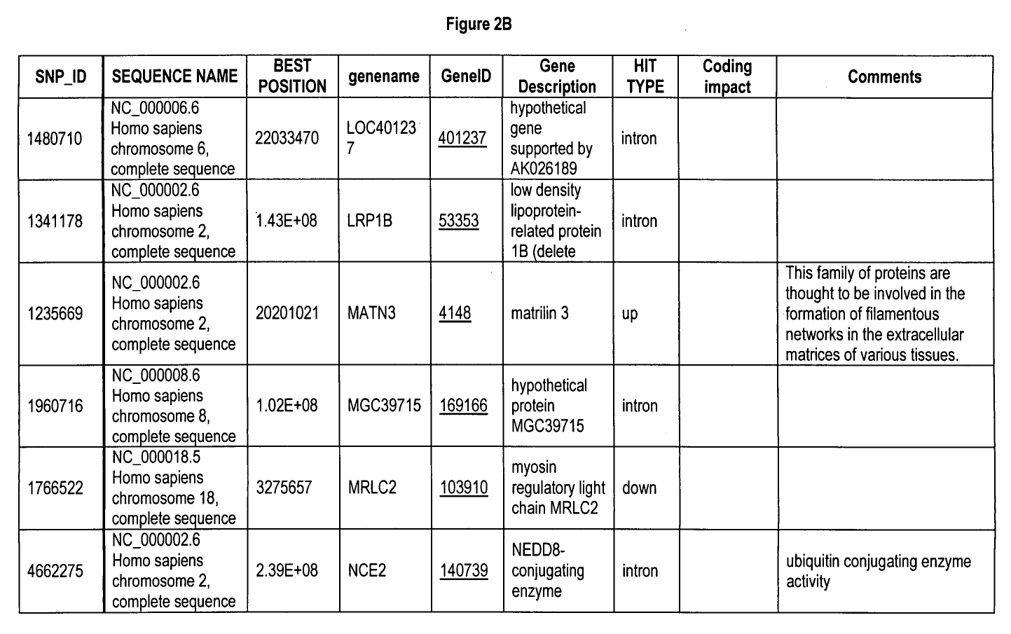

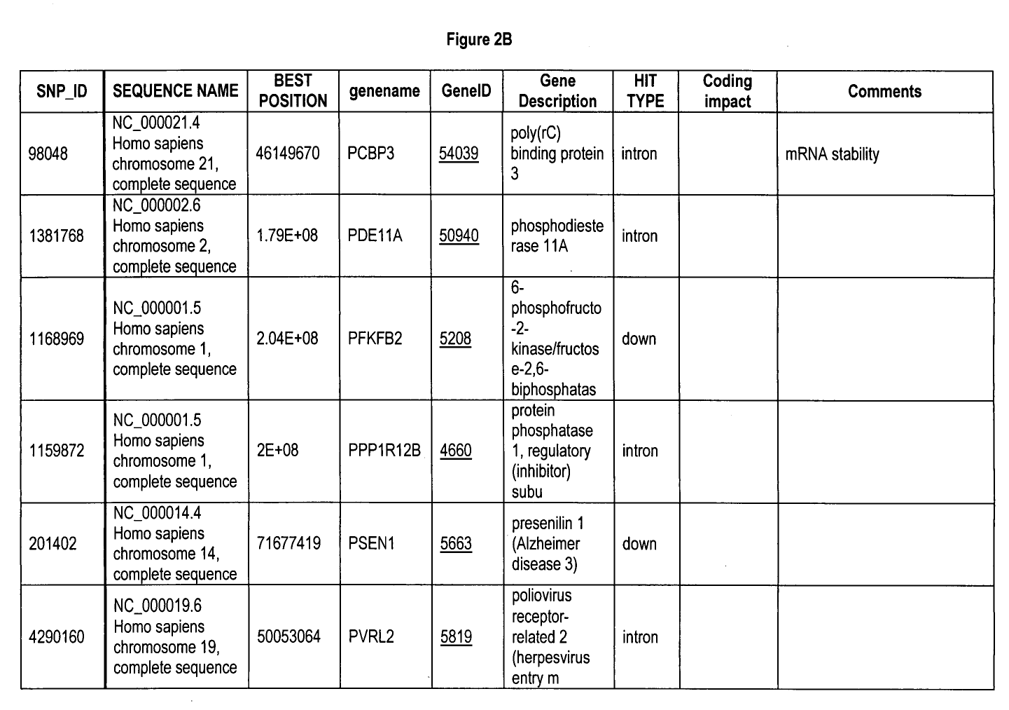

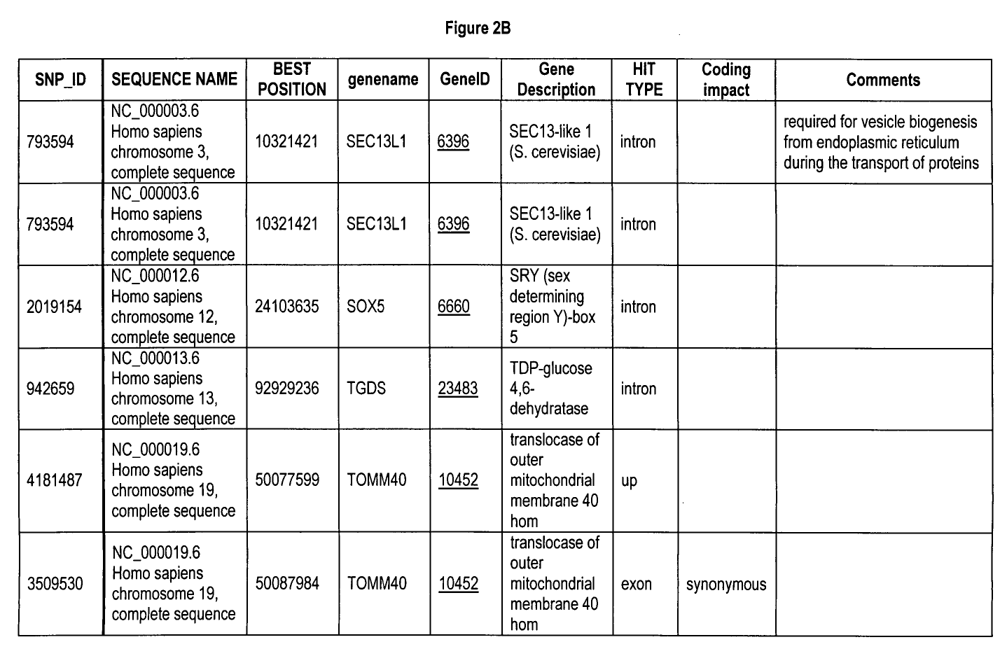

The term “associated gene” or “associated gene region” or “AD gene” refers to a gene, a genomic region 10 kb upstream and 10 kb downstream of such gene, or regulatory regions that modulate the expression of such gene, comprising at least a portion of one of the polymorphic regions identified in FIGS. 1 and 2, and all associated gene products (e.g., isoforms, splicing variants, and/or modifications, derivatives, etc.) The sequence of an AD gene in an individual may contain one or more reference or alternate alleles, may contain a combination of reference and alternate alleles, or may contain alleles in linkage disequilibrium with one or more of the polymorphic regions identified in FIGS. 1 and 2.

The term “associated gene pathway” generally refers to genes and gene products comprising an AD-related disease pathway (i.e., a pathway related to resistance or susceptibility to AD-related disease), and may include one or more genes that act upstream or downstream of an associated gene in an AD-related disease pathway; or any gene whose product interacts with, binds to, competes with, induces, enhances or inhibits, directly or indirectly, the expression or activity of an associated gene; or any gene whose expression or activity is induced, enhanced or inhibited, directly or indirectly, by an associated gene. An associated gene pathway may refer to one or more genes. For example, one of the genes of the invention, LRP1, has the following ligands, which are considered to be in the LRP1 pathway.

| Gene | Gene ID | Gene Description |
|---|---|---|
| LRPAP1 | 104225 | LOW DENSITY LIPOPROTEIN RECEPTOR- |
| | | RELATED PROTEIN-ASSOCIATED PROTEIN |
| | | 1; LRPAP1 |
| PLAU | 191840 | PLASMINOGEN ACTIVATOR, URINARY; |
| | | PLAU |
| PLAT | 173370 | PLASMINOGEN ACTIVATOR, TISSUE |
| PAI1 | 173360 | PLASMINOGEN ACTIVATOR INHIBITOR 1; |
| | | PAI1 |

The term “complementary” can mean partially complementary or completely complementary and generally refers to the natural hydrogen bonding between purine and pyrimidine base pairs. The term “partially complementary” refers to instances where only some of the base pairs are bonded. The term “completely complementary” refers to instances where all or nearly all of the base pairs are bonded. The term “perfectly complementary” refers to instances where all of the base pairs are bonded.

The term “derivative” refers to chemical modification of a nucleic acid, a protein or mimetic thereof. Examples of chemical modifications of a nucleic acid include replacement of hydrogen by an alkyl, an acyl or an amino group. A nucleic acid derivative may also refer to a nucleic acid that was derived from another nucleic acid (e.g., mRNA transcribed from a gene, cDNA synthesized from an RNA molecule, or cRNA synthesized from a DNA molecule, etc.) A nucleic acid derivative can encode a polypeptide that retains, changes, inhibits or enhances essential characteristics or functions of the polypeptide that the natural nucleic acid encodes. A polypeptide derivative is one that is modified by glycosylation, pegylation or other process, and that retains, changes, inhibits or enhances at least one characteristic or function (e.g., immunological response) of the polypeptide from which it was derived.

The term “stringent conditions” refers to conditions for hybridization of complementary nucleic acids wherein the presence of a nucleic acid may be detected. For example, the detection of hybridization may be used as a proxy for determining the presence of a particular nucleic acid. Different stringency conditions may be utilized under different circumstances. Stringent conditions depend on, for example, length of the nucleic acids, hybridization temperature, buffers, and other hybridization reaction conditions. Generally, stringent conditions are selected to be about 5° C. lower than the thermal melting point (Tm) of a specific sequence at a defined ionic strength and pH. The Tm is the temperature (under defined ionic strength, pH and nucleic acid concentration) at which 50% of the complementary nucleic acids hybridize to a target nucleic acid at equilibrium. As target nucleic acids are generally present in excess, at Tm, 50% of the complementary nucleic acids are occupied at equilibrium. Typically, stringent conditions include a salt concentration of at least about 0.01 to 1.0 M Na ion concentration (or other salts) at pH 7.0 to 8.3 and the temperature is at least about 30° C. for short probes (e.g., 10 to 50 nucleotides). Stringent conditions can also be achieved with the addition of destabilizing agents such as formamide. For example, in some embodiments, conditions of 5×SSPE (750 mM NaCl, 50 mM NaPhosphate, 5 mM EDTA, pH 7.4) and a temperature of 25-30° C. are suitable for allele-specific nucleic acid hybridizations. In other embodiments, conditions of 1M TMACl (tetramethylammonium chloride), 3.25 M Tris (pH 7.8-8.0), 0.00325% Triton X-100, and a temperature of 50° C. are suitable for allele-specific nucleic acid hybridizations. Example 16 provides yet another example of conditions appropriate for allele-specific nucleic acid hybridizations.

The terms “isolated” and “purified” refer to a material that is substantially or essentially removed from or concentrated in its natural environment. For example, an isolated nucleic acid is one that is separated from the nucleic acids that normally flank it or from other biological materials (e.g., other nucleic acids, proteins, lipids, cellular components, etc.) in a sample. In another example, a polypeptide is purified if it is substantially removed from or concentrated in its natural environment.

The term “modulate” refers to a change such as in expression, lifespan, or function such as an increase, decrease, alteration, enhancement or inhibition of expression or activity.

The term “nucleic acid,” refers to a deoxyribonucleotide or ribonucleotide, whether singular or in polymers, naturally occurring or non-naturally occurring, double-stranded or single-stranded, translated (e.g., gene) or untranslated (e.g. regulatory region), or any fragments, derivatives, mimetics or complements thereof. A nucleic acid includes analogs (e.g., phosphorothioates, phosphoramidates, methyl phosphonate, chiral-methyl phosphonates, 2-O-methyl ribonucleotides) or modified nucleic acids (e.g., modified backbone residues or linkages) or nucleic acids that are combined with carbohydrates, lipids, protein or other materials, or peptide nucleic acids (PNAs) (e.g., chromatin, ribosomes, transcriptosomes, etc.) A nucleic acid can include one or more polymorphisms, variations or mutations (e.g., SNPs, insertions, deletions, inversions, translocations, etc.) Examples of nucleic acids include oligonucleotides, nucleotides, polynucleotides, nucleic acid sequences, genomic sequences, antisense nucleic acids, DNA regions, probes, primers, genes, regulatory regions, introns, exons, open-reading frames, binding agents, target nucleic acids and allele-specific nucleic acids.

A polymorphic or variant site is a locus of genetic variation in a genome. A polymorphic site is occupied by two or more polymorphic forms (also known as variant forms or alleles). A single nucleotide polymorphic site (SNP) is a variation at a single nucleotide. The term “polymorphism” refers a position in a nucleic acid or polypeptide that possesses the quality or character of occurring in several different forms. A nucleic acid or polypeptide may be naturally or non-naturally polymorphic, e.g., having one or more sequence differences (e.g., additions, deletions and/or substitutions) as compared to a reference sequence. A reference sequence may be based on publicly available information (e.g., the U.C. Santa Cruz Human Genome Browser Gateway (genome.ucsc.edu/cgi-bin/hgGateway) or the NCBI website (www.ncbi.nlm.nih.gov)) or may be determined by a practitioner of the present invention using methods well known in the art (e.g., by sequencing a reference nucleic acid). A nucleic acid polymorphism is characterized by two or more “alleles”, or versions of the nucleic acid sequence. Typically, an allele of a polymorphism that is identical to a reference sequence is referred to as a “reference allele” and an allele of a polymorphism that is different from a reference sequence is referred to as an “alternate allele”, or sometimes a “variant allele”. However, as any two reference sequences may differ at a polymorphic locus, an “alternate allele” may be found in a reference sequence and a “reference allele” may not. Furthermore, the designation of a “reference allele” and an “alternate allele” need not be based on any particular reference sequence and can be performed arbitrarily simply as a means to distinguish between two different alleles of a polymorphism. As such, the designation of alleles provided herein as “reference” or “alternate” should not be construed to indicate that the allele is or is not present in a particular reference sequence. A nucleic acid comprising an alternate allele may be referred to as a “variant nucleic acid”. Nucleic acid polymorphisms include loci within nucleic acids encoding a polypeptide, but which due to the degeneracy of the genetic code are not found in nature. A polypeptide polymorphism is characterized by two or more versions of an amino acid sequence, with a version that is identical to a reference sequence referred to as a “reference polypeptide” and a version that is different from a reference sequence referred to as an “alternate polypeptide” or a “polypeptide variant”. Polypeptide polymorphisms include polypeptides encoded by another locus in the human genome or other organism's genome that have substantial homology, in whole or in part, to the polypeptides provided herein. The term “synonymous polymorphism” refers to a polymorphism in a coding region of a gene for which different alleles of the polymorphism encode an identical amino acid sequence. The term “non-synonymous polymorphism” refers to a polymorphism in a coding region of a gene for which different alleles of the polymorphism encode different amino acid sequences. Non-synonymous polymorphisms may be conservative or non-conservative. A “conservative polymorphism” refers to a non-synonymous polymorphism for which the different amino acid sequences encoded are functionally equivalent. A “non-conservative polymorphism” refers to a non-synonymous polymorphism for which the different amino acid sequences encoded are functionally dissimilar. “Functionally equivalent” as used herein refers to a polypeptide capable of exhibiting a substantially similar activity as another polypeptide.

The terms “polypeptide,” “peptide,” “oligopeptide” and “protein” are used interchangeably to refer to a polymer of amino acids, PNAs or mimetics, of no specific length and to all fragments, isoforms, variants, derivatives and modifications thereof. A polypeptide may be naturally and non-naturally occurring. The term variant when used to describe a polypeptide refers to variations in amino acid sequences as compared to a reference polypeptide sequence, whether or not such variations are encoded by conservative or non-conservative polymorphisms, for example. An amino acid substitution that is encoded by a conservative polymorphism may be referred to as a conservative substitution. Likewise, an amino acid substitution that is encoded by a non-conservative polymorphism may be referred to as a non-conservative substitution. The term modification include tags, labels, post-translational modifications or other chemical or biological modifications. In preferred embodiment a polypeptide is purified.

The term “probes” or “primers” refers to nucleic acids that can hybridize, in whole or in part, in a base-specific manner to a complementary strand. Typically, the term “primer” refers to a single-stranded nucleic acid that acts as a point of initiation of template-directed DNA synthesis (e.g., PCR primers) and the term “probe” refers to a single-stranded nucleic acid designed to hybridize to a target nucleic acid. For example, hybridization of the probe to a sample nucleic acid may be used to purify a target nucleic acid within the sample, or detection of hybridization (or lack thereof) of the probe to a sample nucleic acid may be used to determine the presence (or absence) of a target nucleic acid in the sample. Although single-stranded probes and primers are primarily discussed herein, the present invention is not limited to such probes and primers; double-stranded or partially double-stranded probes or primers are also included.

The term “specific hybridization” refers to the ability of a first nucleic acid to bind, duplex or hybridize to a second nucleic acid in a manner such that the second nucleic acid can be identified or distinguished from other components of a mixture (e.g., cellular extracts, genomic DNA, etc.) In certain embodiments, specific hybridization is performed under stringent conditions.

The term “substrate” refers to any rigid or semi-rigid support to which molecules (e.g., nucleic acids, polypeptides, mimetics) may be bound. Examples of substrates include membranes, filters, chips, slides, wafers, fibers (e.g., optical fibers), magnetic or nonmagnetic beads, gels, capillaries, or other tubing, plates, polymers, and microparticles with a variety of surface forms including wells, trenches, pins, channels and pores, and may be manufactured from various substances, including but not limited to glass, silicon, fused silica, borosilicate, quartz, soda lime glass, a polymeric material (e.g., polyethylene, polycarbonate, polyvinylchloride, polystyrene, and the like) or a combination thereof.

The term “vector” refers to any construct or composition by which the expression, transfer or manipulation of a nucleic acid may be accomplished or facilitated. For example, the term vector can be an artificial chromosome (e.g., BAC, YAC, etc.), cosmid, viral particle, viral nucleic acid, plasmid, or a liposome. For example, in some embodiments a vector is a viral nucleic acid or a plasmid with appropriate transcription/translation control signals. An expression vector is a vector that is designed to promote the expression of one or more nucleic acid inserts.

The term “haplotype block” (also known as a linkage disequilibrium bin) refers to a region of a chromosome that contains one or more polymorphic sites (e.g., 1-10) that tend to be inherited together (i.e., are in linkage disequilibrium) (see Patil, et al., Science, 294:1719-1723 (2001); US 20030186244)). In other words, combinations of polymorphic forms at the polymorphic sites within a block cosegregate in a population more frequently than combinations of polymorphic sites that occur in different haplotype blocks.

The term “haplotype pattern” refers to a combination of polymorphic forms that occupy polymorphic sites, usually SNPs, in a haplotype block on a single DNA strand. For example, the combination of variant forms that occupy all the polymorphisms within a particular haplotype block on a single strand of nucleic acid is collectively referred to as a haplotype pattern of that particular haplotype block. Many haplotype blocks are characterized by four or fewer haplotype patterns in at least 80% of individuals. The identity of a haplotype pattern can often be determined from one or more haplotype determining polymorphic sites without analyzing all polymorphic sites constituting the pattern.

The term “linkage disequilibrium” refers to the preferential segregation of a particular polymorphic form with another polymorphic form at a different chromosomal location more frequently than expected by chance. Linkage disequilibrium can also refer to a situation in which a phenotypic trait displays preferential segregation with a particular polymorphic form or another phenotypic trait more frequently than expected by chance.

The boundaries of a gene are defined by the beginning and end of its transcribed region. A polymorphic site is proximal to a gene if it occurs within the intergenic region between the transcribed region of the gene and that of an adjacent gene. Usually, proximal implies that the polymorphic site occurs closer to the transcribed region of the particular gene that that of an adjacent gene. Typically, proximal implies that a polymorphic site is within 400 kb, and preferably within 10 kb of the transcribed region. Polymorphic sites not occurring in proximal regions as defined above are said to occur in regions that are distal to the gene. If a segment of genomic DNA is said to occur within a certain distance of a polymorphic site, then the most distant point of the segment occurs within the specified distance. Likewise if a segment of genomic DNA is said to occur within a certain distance of a gene, then the most distant point of the segment occurs with the specified distance of closest transcriptional endpoint of the gene.

The term “specific binding” refers to the ability of a first molecule (e.g., an antibody) to bind or duplex to a second molecule (e.g., a polypeptide) in a manner such that the second molecule can be identified or distinguished from other components of a mixture (e.g., cellular extracts, total cellular polypeptides, etc.)

A nonhuman homolog (or cognate form) of a human gene is the gene in a nonhuman species, such as a mouse, that shows greatest sequence identity at the nucleic acid and encoded protein level, and higher order structure and function of the protein product to that of the human gene or encoded product.

Modulation means an increase or decrease in function of a gene product.

Statistically significant” means significant at a p value≦0.05.

The term “comprising” indicates that other elements can be present besides those explicitly stated.

Various embodiments and modifications can be made to the invention disclosed in this application without departing from the scope and spirit of the invention. Unless otherwise apparent from the context any embodiment, feature or element of the invention can be used in combination with any other. Any embodiment, feature or element of the invention described in the alternative to other embodiments, features or elements can be excluded from the invention. Throughout this disclosure various patents, patent applications, gene identifiers, and publications are referenced and unless otherwise indicated, are incorporated by reference in their entirety and for all purposes to the same extent as if so individually denoted.

### DETAILED DESCRIPTION OF THE INVENTION

The invention provides a collection of polymorphic sites having resistance and susceptibility alleles associated with resistance or susceptibility to Alzheimer's disease, particularly the most common form of Alzheimer's disease, known as late-onset Alzheimer's disease (LOAD). The polymorphic sites were identified by analyzing a sampling of polymorphic sites throughout the human genome in a population having late-onset disease and a control population.

The collection of polymorphic sites and the genes in which they occur have a variety of uses. The genes and encoded proteins can be used to identify compounds that modulate the expression or activity of encoded proteins. Such compounds are useful for treatment, prophylaxis, diagnosis or prognosis of Alzheimer's related diseases. The collection of genes is also useful for generating transgenic animal models of Alzheimer's related disease. These models are useful for screening drugs. The polymorphic sites are also useful in profiling individuals for susceptibility to disease, response to therapies, or amenability to treatment.

I AD

Alzheimer's disease (AD) is a progressive degenerative disease. AD mainly occurs late in life. It is estimated that 2-3 percent of the population over 65 and around 10% of the population over 80 suffer from some form of AD. Roughly half of all AD patients have a positive family history.

Images of brains of patients with AD show significant loss of cells and volume in the regions of the brain devoted to memory and higher mental functioning. Moreover, biopsies of AD patients typically reveal twisted nerve cell fibers, known as neurofibrillary tangles and a sticky protein called beta amyloid.

Neurofibrillary tangles are the damaged remains of microtubules, which allow the flow of nutrients through the neurons (nerve cells). A key component in these tangled fibers is an abnormal form of the tau protein, which in its healthy version helps in the assembly and stabilization of the microtubule structure. The defective tau protein appears to block the actions of the normal version.

Beta amyloid (also called Aβ) is the second significant finding in AD biopsies. This insoluble protein accumulates and forms sticky patches called neuritic plaques, which are found surrounded by the debris of dying nerve cells in the brains of Alzheimer's victims.

There are various forms of AD. Early onset AD is a rare form of AD in which people are diagnosed with the disease before age 65. Less than 10% of all AD patients have this type. Early-onset Alzheimer's is strongly hereditary. The hereditary form of early onset AD is also known as familial Alzheimer's disease or FAD. Three genes that have been implicated in early-onset Alzheimer's encode proteins called presenilin 1, presenilin 2, and amyloid precursor protein (APP). The forms of these genes that lead to Alzheimer's are deterministic; virtually everyone who has these forms develops the disease. In other words, the penetrance of certain mutations in these genes is 100%. Each child of a parent who has an Alzheimer-related mutation in one of these genes has a 50 percent chance of inheriting the form that causes Alzheimer's disease. Because of trisomy at chromosome 21, which encodes amyloid precursor protein, people with Down syndrome are particularly at risk of developing a form of early onset AD. Adults with Down syndrome are often in their mid- to late 40s or early 50s when symptoms first appear.

Late-onset AD is the most common form of AD. It usually appears after a person reaches the age of 65. Late-onset AD strikes almost half of all people over the age of 85 and may or may not be hereditary. Late-onset AD is also called sporadic AD. Late-onset Alzheimer's, has a subtler and less clearly understood inheritance pattern. The cholesterol-processing protein called apolipoprotein E (APOE) is a susceptibility gene that occurs in three different alleles: APOE-e4, APOE-e3, and APOE-e2. ApoE-e3 is the most common form and APOE-e2 is the least common. People with one copy of APOE-e4 have an increased chance of developing Alzheimer's, and people with two copies are at even higher risk. However, not everyone with two copies develops Alzheimer's, and many people with the disease have no APOE-e4 at all. In other words, the APOE-e4 allele shows incomplete penetrance. Several other genes identified in the present application also influence the likelihood of developing late-onset Alzheimer's disease.

All embodiments of the invention can be practiced on genes other than APP, PSEN1 and APOE1, or genomic regions in linkage disequilibrium, or variant sites in such genes and regions. However, some embodiments of the invention employ APP, PSEN1 and/or APOE1, other genomic regions in linkage disequilibrium therewith or variant sites in such genes or regions, particularly in combination with other genes of the invention, as described in more detail below.

II AD Nucleic Acids

The invention provides a collection of about 100 variant sites having forms associated with susceptibility or resistance to late-onset Alzheimer's disease. The variants sites occur in the following genes: APP, PSEN1, APOE, APOC, C9orf52, CTNND2, CUGBP1, DKFZP566K1924, FARS1, FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC388110, MGC39715, NCE2, PFKFB2, PPP1R12B, TGD5, and TTLL2. Of these genes, APOE1 has previously been associated with late-onset Alzheimer's disease, and two, APP and PSEN1, have previously been associated with early onset Alzheimer's disease. Some of the other genes LU, PVRL2, TOMM40, APOC1, APOC4, APOC2, CLPTM1 are found on the same chromosome as APOE1. Other genes occur at chromosomal locations not previously known to be associated with Alzheimer's disease.

FIG. 1 shows variant sites in a region of chromosome 19 surrounding APOE. This region includes the genes LU, PVRL2, TOMM40, APOE, APOC1, APOC4, APOC2, and CLPTM1. Some of the variant sites and their surrounding genomic regions have variant forms that are correlated with susceptibility or resistance to AD. At such sites, one variant form occupying the site is correlated with susceptibility to AD and the other to resistance to AD.

FIG. 1, column 1, entitled “REFSNP_ID”, identifies a SNP identification number from dbSNP (NCBI) for each variant This is the reference number according to dbSNP database established and maintained by NCBI of the National Library of Medicine at the National Institute of Health)

FIG. 1, column 2, entitled “SUBSNP_ID” is a submission identifier for Applicants for submission to dsSNP.

FIG. 1, column 3, entitled “SNP_ID” is an internal Perlegen SNP identifier.

FIG. 1, column 4, entitled “SEQUENCE NAME” is the name of sequence to which the SNP maps on NCBI Build 34.

FIG. 1, column 5, entitled “BEST POSITION”, identifies the position for each variant according to Build 34 of the human genome.

FIG. 1, column 6, entitled “CONTIG_POSITION”, identifies position of for each variant in contig from dbSNP according to Build 34 of the human genome.

FIG. 1, column 7, entitled “Gene Name,” identifies the name for the gene or gene region within which the SNP occurs according to NCBI. The gene names are those defined by the authorities in the field such as HUGO, or conventionally used in the art to describe the genes. Reference to a gene includes the sequence of the gene encoded by the Gene Identifer described below and allelic variants thereof.

FIG. 1, column 8, entitled “Gene Identifier” identifies the Genes Identifier in the NCBI's gene database (January 2005 version).

FIG. 1, column 9, entitled “geneDescription” identifies the gene description from NCBI gene database.

FIG. 1, column 10, entitled “HIT_TYPE” identifies where the SNP lies within or relative to a gene. A “HIT_TYPE” of “intron” means that the genetic variant occurred within an intron of a gene. A “HIT_TYPE” of “exon” means that the genetic variant occurred within an exon of a gene. A “HIT_TYPE” of “down” means that the genetic variant occurred within 10 kb downstream of a gene. A “HIT_TYPE” of “up” means that a genetic variant occurred with 10 kb upstream from the start codon. A “HIT_TYPE” of “between” means that a genetic variant occurred at a distance greater than 10 kb from a gene(s).

FIG. 1, column 11 entitled “CALL_RATE” identifies the rate of alleles that are genotyped which passed the Quality Control (QC) criteria (See Examples) from both cases and controls.

FIG. 1, column 12, entitled “CASES_P” identifies the frequency of reference allele in cases.

FIG. 1, column 13, entitled “CTRLS_P” identifies the frequency of reference allele in controls.

FIG. 1, column 14, entitled “DELTA_P” identifies the difference in allele frequency of cases minus controls. The difference in allele frequency for each variant can be calculated according to the methods in, e.g., US20040210400 and U.S. application Ser. No. 10/970,761, filed Oct. 20, 2004, entitled “Improved Analysis Methods and Apparatus for Individual Genotyping”. A positive value for DELTA_P indicates that the reference allele occurs more frequently in the cases than in the controls, and a negative DELTA_P indicates that the reference allele occurs more frequently in the controls than in the cases.

FIG. 1, column 15, entitled “GC_TREND_TEST”, identifies p-value of trend test, corrected by genomic control. This value represents the significance of the DELTA_P value. The lower the value the more significant the association. Thus, for example, the reference form of SNP 2927446 in the gene Lu (col. 1 of FIG. 1) is not significantly associated with resistance or susceptibility to Alzheimer's disease, whereas the reference form of the SNP 440277 in the gene PCRL2 is significantly associated with Alzheimer's disease.

FIG. 1, column 16, entitled “DELTA_PHAT2” identifies the absolute value of the delta p-hat, which is the estimated allele frequency difference calculated from pooled genotyping results. The estimated allele frequency difference for each variant can be calculated according to the methods in, e.g., U.S. App. Ser. No. 60/460,329, filed Apr. 3, 2003, entitled “Apparatus and Methods for Analyzing and Characterizing Nucleic Acid Sequences”; and US2005001978. Briefly, the difference in estimated allele frequency is equal to the relative allele frequency in a case group minus the relative allele frequency in a control group. The relative allele frequency (P′, or PHAT, or P-hat) can be calculated according to the following equation:
P′=I_{reference}/(Iₐₗₜₑᵣₙₐₜₑ+I_{reference}),
where “I” indicated an intensity measurement from, e.g., a microarray. As for DELTA_P, A positive value for DELTA_PHAT2 indicates that the reference allele occurs more frequently in the cases than in the controls, and a negative DELTA_PHAT2 indicates that the reference allele occurs more frequently in the controls than in the cases.

FIG. 1, column 17, entitled “STD_ERR_PHAT2”, identifies the standard error of the estimated allele frequency difference from pooled genotyping.

FIG. 1, column 18, entitled “EMPERICAL_P_VALUE_SO”, identifies the empirical p-value of the estimated allele frequency difference from pooled genotyping, corrected by addition of a factor to minimize the coefficient of variance of the t-test. This value represents the significance of the DELTA_PHAT2.

FIG. 1, column 19, entitled “Coding Impact”, identifies whether the variant influences protein structure and therefore is only entered for exonic variants.

As is illustrated by FIG. 1, variants appear either between genes or in associated gene regions. Gene regions that appear to be related to AD include, but are not limited to those encoding: LU, PVRL2, TOMM40, APOE, APOC1, APOC4, APOC2, and CLPTM1.

LU, located on chromosome 19, at 19q13.2, encodes is a Lutheran blood group transmembrane receptor protein and is the most critical receptor mediating adhesion to laminin under both static and flow conditions in sickle cell anemia.

PVRL2 or poliovirus receptor-related 2 is also located on chromosome 19, at 19q13.2-q13.4, and this gene encodes an adhesion molecule widely expressed in cell lines of different lineages, including hematopoietic, neuronal, endothelial and epithelial cells. In 1995, it was identified and reported in humans. Later it was identified as a transmembrane molecule related to CD155 and named Poliovirus Receptor Related2 (PRR2). It belongs to a family of immunoglobulin-like molecules that includes four members (CD111 , CD112, PRR3 and CD155) sharing an ectodomain made of three Ig domains, of V, C, C types. PVRL2 encodes 2 different transmembrane isoforms sharing identical ectodomains but different transmembrane and cytoplasmic regions. The two corresponding transcripts of 4.4 and 3.0 kb were detectable in several tissues. PVRL2 is expressed in the myelo-monocytic and megakaryocytic hematopoietic lineages and the function in hematopoiesis is currently unknown. PVRL2 is an intercellular homophilic adhesion molecule also known as nectin 2. Homophilic adhesion correlates with the tyrosine phosphorylation of the long isoform. PVRL2 localizes specifically at adherens junctions via its cytoplasmic interaction with the scaffold F-actin binding protein afadin. This interaction is mediated by a sequence located to the C terminal ends of both isoforms of PVRL2 (A/ExYV) and the PDZ domain of afadin. This sequence is also found in PVRL2 and PRR3 and represents a specific consensus in the family, which is also named nectin family. Disruption of the murine PVRL2gene leads to infertility of male mice with morphologically aberrant spermatozoa. PVRL2 mediates entry of some alpha-herpesvirus mutants (also named HveB) via its V domain. PVRL2 is involved in cell to cell spreading of the virus.

TOMM40, located also on chromosome 19, at 19q13, is a gene thought to be a translocase of outer mitochondrial membrane 40 and used in the import of protein precursors into the mitochondria. Suzuki, H., J Biol Chem. Dec. 1, 2000;275(48):37930-6. TOMM40 gene products have been found to be expressed in lymph and pancreas cells.

APOE is an important apoprotein of the chylomicron and binds to a specific receptor on liver cells and peripheral cells. APOE is essential for the normal catabolism of triglyceride-rich lipoprotein constituents. The apoE gene is mapped at 19q13.2 in a cluster with apoC1 and apoC2. Defects in APOE result in familial dysbetalipoproteinemia, or type III hyperlipoproteinemia (HLP III), in which increased plasma cholesterol and triglycerides are the consequence of impaired clearance of chylomicron and very low density lipoprotein remnants.

APOC1 or apolipoprotein C1, located at chromosome 19 at 19q13.2, is a protein encoded by a member of the apolipoprotein C1 family. This protein is expressed primarily in the liver, and it is activated when monocytes differentiate into macrophages. A pseudogene of the apoc1 gene is located 4 kb downstream in the same orientation, on the same chromosome. This gene is mapped to chromosome 19, where it resides within a apolipoprotein gene cluster. Alternatively spliced transcript variants have been found for this gene, but the biological validity of some variants has not been determined.

APOC4 is encoded by apolipoprotein (apo)C4 gene located at 19q13.2, and which is a member of the apolipoprotein gene family. It is expressed in the liver and has a predicted protein structure characteristic of the other genes in this family. Apo C4 is a 3.3-kb gene consisting of 3 exons and 2 introns; it is located 0.5 kb 5′ to the apoC2 gene.

APOC2 is encoded by the apoc2 gene located at 19q13.2, and is secreted in plasma where it is a component of very low density lipoprotein. This protein activates the enzyme lipoprotein lipase, which hydrolyzes triglycerides and thus provides free fatty acids for cells. Mutations in this gene cause hyperlipoproteinemia type IB, characterized by hypertriglyceridemia, xanthomas, and increased risk of pancreatitis and early atherosclerosis.

Additional examples of variant sites having variant forms that are related to resistance or susceptibility to AD are identified in FIG. 2. These variant sites were identified by association studies using a collection of variant sites throughout the genome as described in the Examples.

FIG. 2, column 1, entitled “SNP_ID”, is an internal Perlegen number that identifies a single variant position.

FIG. 2, column 2, entitled “DELTA_P”, identifies the difference in allele frequency of the reference allele of cases minus controls.

FIG. 2, column 3, entitled “HWE_P_VALUE_CTRLS”, identifies the p-value indicating significance of deviation from the Hardy-Weinberg Equilibrium (tested in controls only). All of the variant sites shown in FIG. 2 have variant forms significantly associated with resistance or susceptibility to AD.

FIG. 2, column 4, entitled “CALL_RATE,” identifies the rate of alleles that are genotyped that passed the Quality Control (QC) criteria (See Examples) from both cases and controls.

FIG. 2, column 5, entitled “GC_TREND_TEST” identifies p-value of trend test, corrected by genomic control, and represents the significance of the DELTA_P.

FIG. 2, column 6, entitled “SEQUENCE_NAME” identifies the name of the sequence to which the SNP maps according to NCBI Build 34.

FIG. 2, column 7, entitled “BEST_POSITION” identifies the position for each variant according to Build 34 of the human genome.

FIG. 2, column 8, entitled “genename” identifies the name for the gene or gene region within which the SNP occurs (according to NCBI).

FIG. 2, column 9, entitled “Gene Identifier” identifies the hyperlink identifier of the gene in the NCBI gene database.

FIG. 2, column 10, entitled “gene description” provides a brief description from NCBI of the gene named in column 8.

FIG. 2, column 11, entitled “HIT_TYPE” identifies where the SNP lies within or relative to a gene. A “HIT_TYPE” of “intron” means that the genetic variant occurred within an intron of a gene. A “HIT_TYPE” of “exon” means that the genetic variant occurred within an exon of a gene. A “HIT_TYPE” of “down” means that the genetic variant occurred within 10 kb downstream of a gene. A “HIT_TYPE” of “up” means that a genetic variant occurred with 10 kb upstream from the start codon. A “HIT_TYPE” of “between” means that a genetic variant occurred at a distance greater than 10 kb from a gene(s).

FIG. 2, column 12, entitled “Coding impact” identifies whether the variant influences protein structure and therefore is only entered for exonic variants.

FIG. 2, column 13, entitled “comments” provides comments on the function of certain genes.

Thus, FIG. 2 illustrates additional variant sites as well as associated gene regions having variant forms associated with resistance or susceptibility to AD. Such associated gene regions include: a2bp1, ahsg, apoE, app, c9orf52, cacna1c, ckm, ctnnd2, cugbp1, dkfzp566k1924, fars1, fg12, flj14442, flj36760, flj38736, kiaa1486, kiaa1862, laptm4a, lnx2, loc147468, loc166522, loc283867, loc387711, loc388110, loc401237, lrp1B, matn3, mgc39715, mrlc2, nce2, pcbp3, pde11A, pfkfb2, ppp1r12b, psen1, pvrl2, sec13L1, sox5, tgds, tomm40, ttll2 and any fragments or derivatives thereof.

Additional variants (and their associated gene regions) that can be used to diagnose, treat, or prevent AD include, but are not limited to, those in haplotype blocks with the variants identified in FIGS. 1, 2 and 3. Such variants can be identified according to, e.g., U.S. Ser. No. 10/106,097, entitled “Methods For Genomic Analysis”, filed Mar. 26, 2002; U.S. Ser. No. 10/284,444, filed Oct. 31, 2002, entitled “Human Genomic Polymorphisms”; and Patil, N. et al, “Blocks of Limited Haplotype Diversity Revealed by High-Resolution Scanning of Human Chromosome 21” Science 294, 1719-1723 (2001). A variant in a haplotype block with a variant of FIG. 1, 2 or 3 that is associated with AD is also associated with AD. More specifically, an allele of a variant in a haplotype pattern with an allele of a variant of FIG. 1, 2 or 3 that is associated with resistance to AD is also associated with resistance to AD. Similarly, an allele of a variant in a haplotype pattern with an allele of a variant associated with susceptibility to AD identified in FIG. 1, 2 or 3 is also associated with susceptibility to AD.

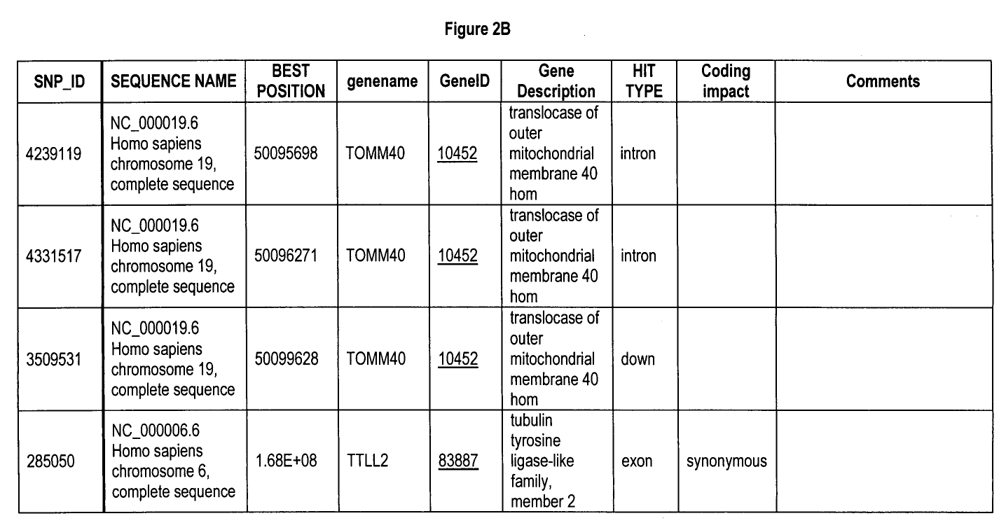

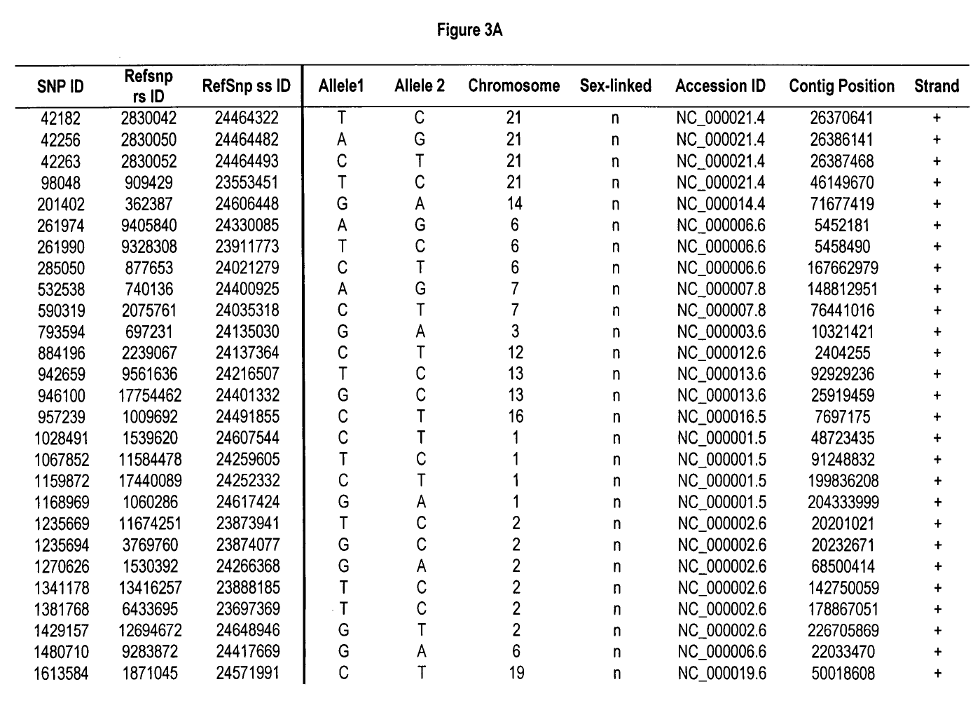

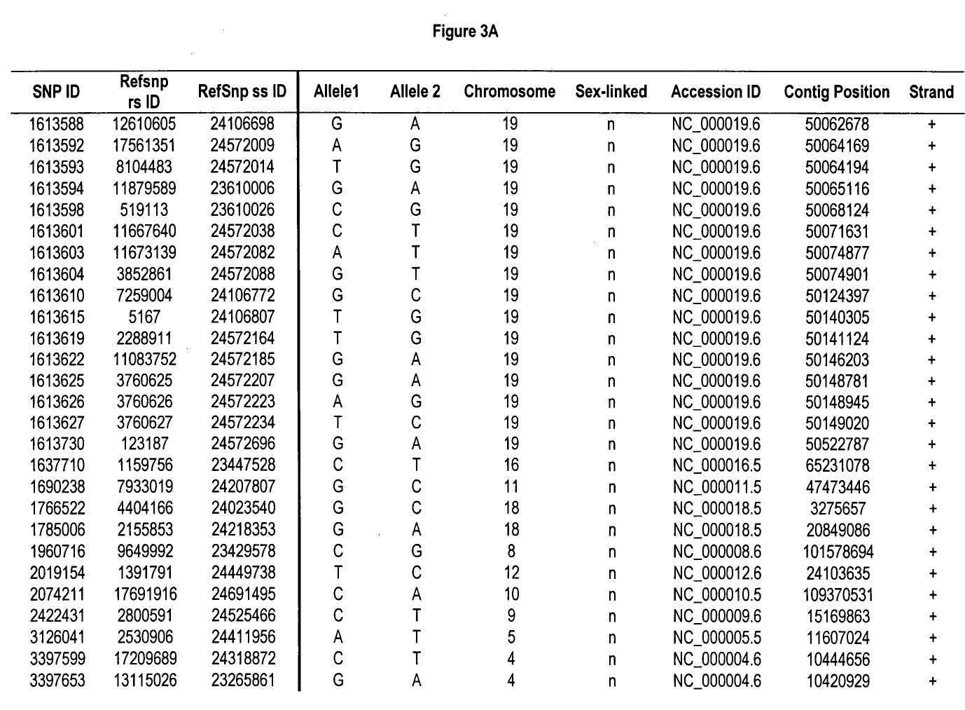

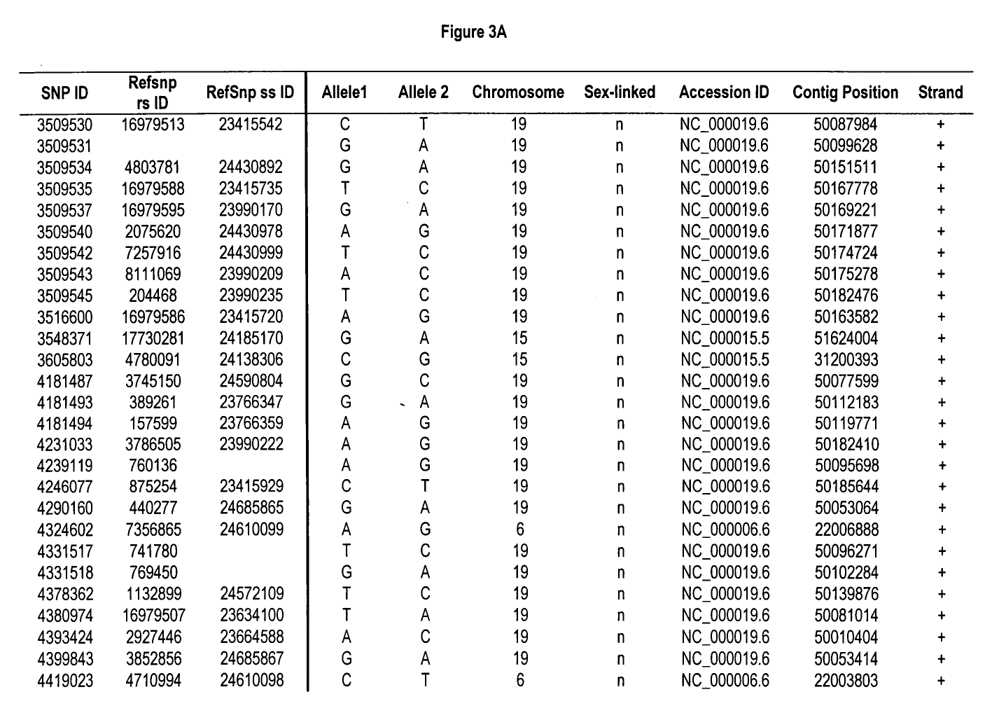

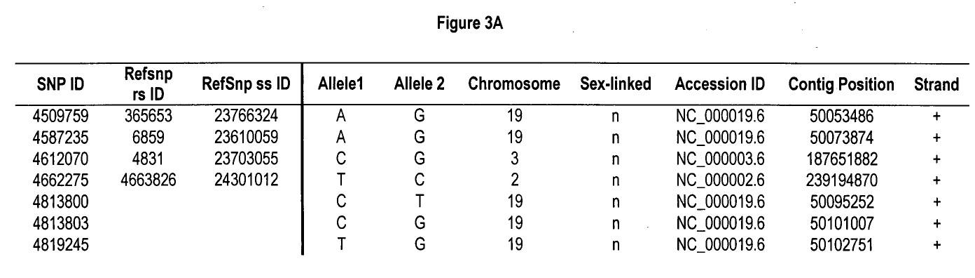

FIG. 3 provides additional information pertaining to the variant sites shown in FIGS. 1 and 2. having variant forms associated with resistance or susceptibility to Alzheimer's disease

FIG. 3, column 1, entitled “SNP_ID”, provides an internal Perlegen number that identifies a single variant position. This same numbering is used in FIGS. 1 and 2.

FIG. 3, column 2, entitled “RefSnp_rsID” provides the RefSNP rsID from NCBI, if available. This same numbering is used in FIG. 1

FIG. 3, column 3, entitled “RefSnp_ssID” provides the RefSnp ssID for SNPs that Perlegen submitted to dbSNP, if available. This same numbering is used in FIG. 1. If a SNP has an RS_ID but not an SS_ID, this means that Perlegen Sciences has not submitted this SNP to dbSNP, but an existing SNP in dbSNP maps (in the Perlegen alignment process) to the same location as the Perlegen SNP.

FIG. 3, column 4, entitled “Allele 1” provides the nucleotide code for reference alleles and FIG. 3, column 5, entitled “Allele 2” provides the nucleotide code for alternate allele. The designation of reference and alternate alleles is arbitrary as far as resistance or susceptibility to AD is concerned. However, such can be determined by looking up the DELTA P value for a given SNP in FIG. 2, column 2. If DELTA P is positive, the reference allele is associated with susceptibility to AD, and if DELTA P is negative, the reference allele is associated with resistance to AD. Conversely, if DELTA P is positive, the alternate allele is associated with resistance to AD, and if DELTA P is negative, the alternate allele is associated with susceptibility to AD.

FIG. 3, column 6, entitled “Chromosome” provides the chromosome number of the NCBI Build 34 contig on which the best alignment was found. X symbolizes the X chromosome. Y symbolizes the Y chromosome. U symbolizes sequences not assigned to any chromosome on Build 34. This field may be null if a SNP could not be placed on any contig.

FIG. 3, column 7, entitled “sex-linked” provides information on sex-linkage of the SNP. “n” represents an autosomal SNP; “P” represents a pseudoautosomal SNP (e.g., on X or Y chromosomes in the pseudoautosomal region); “S” represents a sex-linked SNP (either on the X or Y chromosome, but not in the pseudoautosomal region); and U represents an unassigned (or unknown pseudoautosomal status for X and Y).

FIG. 3, column 8, entitled “accession ID” represents the accession number from NCBI Build 34 of the contig to which the SNP aligns. This may be null.

FIG. 3, column 9, entitled “Contig Position” represents the nucleotide position in NCBI Build 34 contig of the reference base in the alignment. This may be null.

FIG. 3, column 10, entitled “Strand” is a + or −, based on the strand for Allele 1 on NCBI Build 34. This may be null.

FIG. 3, column 11, entitled “Assayed sequence” is the 29-mer that was used to assay the SNP on a microarray, with an ambiguity character representing the SNP at the middle base (base 15).

The genes showing the strongest associations with resistance or susceptibility to Alzheimer's disease are shown in Table 9.

TABLE 9

| Gene ID in NCBI Gene Database | Gene Name |
|---|---|
| 341 | APOC1 |
| 348 | APOE |
| 351 | APP |
| 775 | CACNA1C |
| 1158 | CKM |
| 10667 | FARS1 |
| 57624 | KIAA1486 |
| 147468 | LOC147468 |
| 166522 | LOC166522 |
| 169166 | MGC39715 |
| 140739 | NCE2 |
| 54039 | PCBP3 |
| 50940 | PDE11A |
| 5208 | PFKFB2 |
| 5819 | PVRL2 |
| 6396 | SEC13L1 |
| 10452 | TOMM40 |
| 83887 | TTLL2 |

The preferred polymorphic sites in or around these genes are shown in Table 10.

| Genomic Location | | | Location with | | |
|---|---|---|---|---|---|
| (NCBI Build 35) | | | respect to gene | | |
| Chr | Position | Gene | transcript | Gene Description | Synonymous |
| 1 | 203638695 | PFKFB2 | down | 6-phosphofructo-2-kinase/ | |
| | | | | fructose-2,6-biphosphatas | |
| 2 | 238690120 | NCE2 | intron | NEDD8-conjugating enzyme | |
| 2 | 178490014 | PDE11A | intron | phosphodiesterase 11A | |
| 2 | 226328835 | KIAA1486 | intron | KIAA1486 protein | |
| 3 | 10321421 | SEC13L1 | intron | SEC13-like 1 (S. cerevisiae) | |
| 4 | 10370042 | LOC166522 | up | similar to MIST | |
| 4 | 10346315 | LOC166522 | intron | similar to MIST | |
| 6 | 5452181 | FARS1 | intron | phenylalanine-tRNA synthetase 1 | |
| | | | | (mitochondrial) | |
| 6 | 167724102 | TTLL2 | exon | tubulin tyrosine ligase-like | yes |
| | | | | family, member 2 | |
| 6 | 5458490 | FARS1 | intron | phenylalanine-tRNA synthetase 1 | |
| | | | | (mitochondrial) | |
| 8 | 101691287 | MGC39715 | intron | hypothetical protein MGC39715 | |
| 12 | 2404255 | CACNA1C | intron | calcium channel, voltage- | no |
| | | | | dependent, L type, alpha | |
| 18 | 20851097 | LOC147468 | exon | similar to WW domain binding | |
| | | | | protein 2 | |
| 19 | 50087984 | TOMM40 | exon | translocase of outer mitochondrial | yes |
| | | | | membrane 40 hom | |
| 19 | 50099628 | TOMM40 | down | translocase of outer mitochondrial | |
| | | | | membrane 40 hom | |
| 19 | 50102284 | APOC1 | up | apolipoprotein C-I | |
| 19 | 50096271 | APOE | up | apolipoprotein E | |
| 19 | 50095698 | APOE | up | apolipoprotein E | |
| 19 | 50053064 | PVRL2 | intron | poliovirus receptor-related 2 | |
| | | | | (herpesvirus entry m | |
| 19 | 50522787 | CKM | up | creatine kinase, muscle | |
| 21 | 46117896 | PCBP3 | intron | poly(rC) binding protein 3 | |
| 21 | 26370641 | APP | intron | amyloidbeta (A4) precursor | |
| | | | | protein (protease nexi | |
| 21 | 26387468 | APP | intron | amyloid beta (A4) precursor | |
| | | | | protein (protease nexi | |

The polymorphisms, alleles and associated genomic regions identified herein can be used to identify, isolate and amplify nucleic acids associated with resistance or susceptibility to AD. Such nucleic acids can be used for prognostics, diagnostics, theranostics, prevention, treatment and further study of AD.

For example, in one embodiment, an AD nucleic acid is a nucleotide sequence from the human genome that comprises a nucleic acid in a position identified in FIG. 1, 2 or 3 or a nucleotide acid in haplotype block or pattern with a nucleic acid position identified in FIG. 1, 2 or 3. Such AD nucleic acids can include coding sequence and/or non-coding sequence. It can comprise, consist essentially of, or consist of the exon or intron encompassing such position. It can be of variable length. In some embodiments such a nucleic acid can be less than 500,000, 100,000, 50,000, 10,000, 5,000, 1,000, 500, 100, 10 or 5 nucleotides in length. In some embodiments such a nucleic acid can be greater than 5, 10, 50, 100, 300, 600, 900, 1,000, 3,000, 6,000, 9,000, 10,000, 30,000, 60,000, 90,000, 100,000, 300,000, 600,000, or 900,000 nucleotides in length.

In one embodiment, an AD nucleic acid is one that can specifically hybridize to an associated genomic region encompassing a nucleic acid position identified in FIG. 1, 2 or 3 or an associated genomic region comprising a nucleic acid in a haplotype block or pattern with a position identified in FIG. 1, 2 or 3. In one embodiment, nucleic acids disclosed herein that can specifically hybridize to a genomic region associated with an AD-related disease, are identified in FIG. 3. Due to the duplex nature of DNA, sequences complementary to those provided in Figure can also specifically hybridize to a genomic region associated with PD-related disease and are contemplated to be part of the instant invention. Thus, nucleic acids provided herein or complementary sequences thereto can, in some embodiments, specifically hybridize to a genomic sequence having one or more polymorphisms identified in FIG. 1, 2 or 3 and/or other polymorphisms in the same haplotype blocks as the polymorphisms in FIG. 1 2 and/or 3. Methods for identifying polymorphisms in a haplotype block and in haplotype patterns within a haplotype block are provided in U.S. Ser. No. 10/106,097 entitled “Methods For Genomic Analysis,” filed Mar. 26, 2002; U.S. Ser. No. 10/284,444, filed Oct. 31, 2002, entitled “Human Genomic Polymorphisms”; in US 20040023275; in U.S. patent application Ser. No. 10/367,558, filed Feb. 14, 2003, entitled “Identifying SNP Patterns” (all of which are assigned to the same assignee as the present application); and in Patil, et al. (2001) “Blocks of Limited Haplotype Diversity Revealed by High-Resolution Scanning of Human Chromosome 21”, Science 294:1719-1723.

In some embodiments, the nucleic acids herein are associated with AD. Nucleic acids associated with resistance to AD comprise at least one allele associated with resistance to AD or an allele in a haplotype pattern with an allele associated with resistance to AD. Nucleic acids associated with susceptibility to AD comprise at least one allele associated with susceptibility to AD or an allele in a haplotype pattern with an allele associated with susceptibility to AD. In some embodiments, a nucleic acid associated with resistance to AD is one that is expressed differently in individuals having a phenotype of resistance to AD as compared to individuals having who do not have a phenotype of resistance to AD, or a nucleic acid having one or more alleles associated with resistance to AD. For example, a nucleic acid associated with resistance to AD is one that can specifically hybridize to a genomic region having one or more alleles of polymorphisms identified in FIG. 1 or 2 as being associated with resistance to AD, or one or more alleles in a haplotype pattern therewith. In other embodiments, a nucleic acid associated with susceptibility to AD is one that is expressed differently in individuals having a phenotype of susceptibility to AD as compared to individuals having who do not have a phenotype of susceptibility to AD, or a nucleic acid having one or more alleles associated with susceptibility to AD. For example, a nucleic acid associated with susceptibility to AD is one that can specifically hybridize to a genomic region having one or more alleles of polymorphisms identified in FIG. 1 or 2 as being associated with susceptibility to AD, or one or more alleles in a haplotype pattern therewith.

In certain embodiments, a set of nucleic acids is provided that can specifically hybridize to at least 2 polymorphisms, preferably at least 3 polymorphisms, at least 4 polymorphisms, at least 5 polymorphisms, at least 6 polymorphisms, at least 7 polymorphisms, at least 8 polymorphisms, or at least 9 polymorphisms associated with AD-related disease such as those identified in FIG. 1 or 2, and/or polymorphisms in haplotype blocks therewith, or complementary sequences thereto. In other embodiments, a set of nucleic acids is provided that can specifically hybridize to at least 2 alleles, preferably at least 3 alleles, at least 4 alleles, at least 5 alleles, at least 6 alleles, at least 7 alleles, at least 8 alleles, or at least 9 alleles associated with resistance to AD-related disease, and/or alleles in haplotype patterns therewith, or complementary sequences thereto. Similarly, a set of nucleic acids may be provided that can specifically hybridize to at least 2 alleles, preferably at least 3 alleles, at least 4 alleles, at least 5 alleles, at least 6 alleles, at least 7 alleles, at least 8 alleles, or at least 9 alleles associated with susceptibility to AD-related disease, and/or alleles in haplotype patterns therewith, or complementary sequences thereto.

A nucleic acid can be single stranded or double stranded. It can also comprise coding (e.g., exon) or non-coding sequence (e.g., introns, 3′ or 5′ untranslated regions, and regulatory regions) or a combination of coding and non-coding nucleic acids. In a preferred embodiment, a coding AD nucleic acid is one that can specifically hybridize to at least a portion of the coding region of an associated gene, or to one or more exons of an associated gene, or to one or more open reading frames of an associated gene.

A nucleic acid provided herein can be fused to at least one other nucleic acid (e.g., a tag sequence or reporter gene) to create a construct for producing a specific protein product, such as a fusion protein. A tag sequence encodes a polypeptide that can assist in isolation or purification of the protein product (e.g., glutathione S transferase (GST) fusion protein or a hemagglutinin A (HA) polypeptide). A reporter gene also encodes an easily assayed protein and is often used to replace other coding regions whose protein products are difficult to assay. A fusion protein is formed by the expression of a hybrid nucleic acid made by combining two nucleic acid sequences.

Conditions for nucleic acid hybridization vary depending on the buffers used, length of nucleic acids, ionic strength, temperature, etc. The term “stringent conditions” for hybridization refers to the incubation and wash conditions (e.g., conditions of temperature and buffer concentration) that permit hybridization of a first nucleic acid to a second nucleic acid. The first nucleic acid may be perfectly (e.g. 100%) complementary to the second or may share some degree of complementarity, which is less than perfect (e.g., more than 70%, 75%, 85%, or 95%). For example, certain high stringency conditions can be used which distinguish perfectly complementary nucleic acids from those less complementary, even those having only a single base mismatch. High stringency, moderate stringency and low stringency conditions for nucleic acid hybridization are known in the art. Ausubel, F. M. et al., “Current Protocols in Molecular Biology” (John Wiley & Sons 1998), pages 2.10.1-2.10.16; 6.3.1-6.3.6. The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90% or at least about 95% or more identical to each other remain hybridized to one another. By varying hybridization conditions from a level of stringency at which no hybridization occurs to a level at which hybridization is observed, conditions which will allow a given sequence to hybridize (e.g., selectively) with the most similar sequences in the sample can be determined. Exemplary conditions are described in Krause, et al., Methods in Enzymology, (1991) 200:546-556 and in Ausubel, et al., “Current Protocols in Molecular Biology”, (John Wiley & Sons 1998), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatches among the sequences that hybridize. Generally, doubling the concentration of SSC results in an increase in TM of ˜17° C. Using these guidelines, the washing temperature can be determined empirically for high, moderate or low stringency, depending on the level of mismatch sought. For example, a low stringency wash can comprise washing in a solution containing 0.2×SSC/0.1% SDS for 10 min at room temperature; a moderate stringency wash can comprise washing in a prewarmed solution (42° C.) solution containing 0.2×SSC/0.1% SDS for 15 min at 42° C.; and a high stringency wash can comprise washing in prewarmed (68° C.) solution containing 0.1×SSC/0.1% SDS for 15 min at 68° C. Furthermore, washes can be performed repeatedly or sequentially to obtain a desired result as known in the art. Equivalent conditions can be determined by varying one or more of the parameters given as an example, as known in the art, while maintaining a similar degree of identity or similarity between the target nucleic acid and the primer or probe used.

Furthermore, a nucleic acid is preferably isolated. Various nucleic acid isolation techniques are well known in the art, such as those described in Sambrook, et al., Molecular Cloning: A Laboratory Manual (Cold Spring Harbor Laboratory, New York) (1989), and Ausubel, et al., Current Protocols in Molecular Biology (John Wiley and Sons, New York) (1997). For example, an isolated nucleic acid is one that is separated from the nucleic acids that normally flank it or from other biological materials (e.g., other nucleic acids, proteins, lipids, cellular components, etc.) in a sample.

Nucleic acids may also be amplified using polymerase chain reaction (PCR) and other techniques known in the art. See Erlich, H. A., “PCR Technology: Principles and Applications for DNA Amplification” (ed. Freeman Press, NY, N.Y., 1992); Innis M. A., et al., “PCR Protocols: A Guide to Methods and Applications” (Eds. Academic Press, San Diego, Calif., 1990). In addition to PCR, other suitable isolation and amplification methods include, for example, the ligase chain reaction (LCR) (see Wu and Wallace, Genomics, 4:560 (1989), Landegren et al., Science, 241:1077 (1988), transcription amplification (Kwoh et al., Proc. Natl. Acad. Sci. USA, 86:1173 (1989)), self-sustained sequence replication (Guatelli et al., Proc. Natl. Acad. Sci. USA, 87:1874 (1990)) and nucleic acid based sequence amplification (NASBA). The latter two amplification methods involve isothermal reactions based on isothermal transcription that produces both single-stranded RNA (ssRNA) and double-stranded DNA (dsDNA) as the amplified products in a ratio of approximately 30-100 fold more ssRNA than dsDNA. Amplification methods may result in a subset of sequences being selected from a complex sample, such as those described in U.S. Ser. No. [unassigned], docket no. 100/1066-10, filed Feb. 14, 2005, entitled “Selection Probe Amplification”.

Further, homologues of the AD nucleic acids presented herein may be present in other species, and may be identified and readily isolated without undue experimentation by molecular biological techniques well known in the art using the polymorphisms, alleles and associated genomic regions identified herein. Further, there may exist nucleic acids at other locations within the genome that encode proteins that have extensive homology to one or more domains of the AD polypeptides herein. These nucleic acids may be identified via similar techniques.

For example, an AD nucleic acid may be labeled and used to screen a genomic or cDNA library constructed from mRNA obtained from the organism of interest. Hybridization conditions will be of a lower stringency when the cDNA library was derived from an organism different from the type of organism from which the labeled nucleic acid was derived. Such lower stringency conditions vary predictably depending on the specific organisms from which the library and the labeled nucleic acids are derived. For guidance regarding such conditions see, for example, Sambrook et al. (1989) Molecular Cloning, A Laboratory Manual, Cold Spring Harbor Press, N.Y.; and Ausubel et al. (1989) Current Protocols in Molecular Biology, Green Publishing Associates and Wiley Interscience, N.Y.

1. Probes and Primers

The nucleic acids herein can be used as probes and primers in various assays. The terms “probe(s)” and “primer(s)” refer to nucleic acids that hybridize, in whole or in part, in a sequence-specific manner to a complementary strand. Probes and primers include polypeptide nucleic acids, such as those described in Nielsen et al. (1991) Science 254:1497-1500.

In certain embodiments, the term “primer” refers to a single-stranded nucleic acid that can act as a point of initiation of template-directed DNA synthesis, such as in PCR. PCR reactions can be designed based on the human genome sequence and the associated genomic regions or polymorphisms identified in Table 1. For example, where a polymorphism is located in an exon, the exon can be isolated and amplified using primers that are complementary to the nucleotide sequences at both ends of the exon. Similarly, where a polymorphism is located in an intron, the entire intron can be isolated and amplified using primers that are complementary to the nucleotide sequences at both ends of the intron. Short- or long-range PCR primers may be designed to amplify the associated genomic regions or polymorphisms identified in Table 1 using methods known in the art and further described in U.S. Pat. No. 6,898,531, US 120030108919, and Ser. No. 10/341,832, filed Jan. 14, 2003, entitled “Apparatus and Methods for Selecting PCR Primer Pairs”.

In some embodiments, a probe or primer contains a region of at least about 10 contiguous nucleotides, preferably at least about 15 contiguous nucleotides, more preferably about 20 or about 30 or about 50 contiguous nucleotides, that can specifically hybridize to a complementary nucleic acid sequence. In addition, a probe or primer is preferably about 100 or fewer nucleotides, more preferably between 6 and 50 nucleotides, and more preferably between 12 and 30 nucleotides in length. In certain embodiments, a first portion of a probe or primer is perfectly complementary to a target nucleic acid, and a second portion of the probe or primer is not perfectly complementary to the target nucleic acid. In some aspects, the portion that is not perfectly complementary contains a binding site, e.g., for a polypeptide or another probe or primer.

To isolate, amplify and/or detect the presence of an AD nucleic acid, a probe or primer or set of such probes or primers or a combination thereof may include at least 1 polymorphism, preferably at least 2 polymorphisms, more preferably at least 3 polymorphisms, or more preferably at least 4 polymorphisms associated with AD-related disease as shown in FIG. 1 or 2, complementary sequences thereto, or polymorphisms that are genetically linked to the polymorphisms in FIG. 1 or 2 (e.g. in the same haplotype block). To isolate, amplify and/or detect the presence of a nucleic acid associated with resistance to AD-related disease, a probe or primer or set of such probes or primers may include at least 1 allele, preferably at least 2 alleles, more preferably at least 3 alleles, or more preferably at least 4 alleles associated with resistance to AD-related disease as shown in FIG. 1 or 2, complementary sequences thereto, or alleles that are genetically linked to the alleles in FIG. 1 or 2 (e.g. in the same haplotype pattern). To isolate, amplify and/or detect the presence of a nucleic acid associated with susceptibility to AD-related disease, a probe or primer or set thereof preferably includes at least 1 allele, preferably at least 2 alleles, more preferably at least 3 alleles, or more preferably at least 4 alleles associated with susceptibility to AD-related disease as shown in FIG. 1 or 2, complementary sequences thereto, or alleles that are genetically linked to the alleles in FIG. 1 or 2 (e.g. in the same haplotype pattern).

In one embodiment, a probe or primer is at least about 70% identical to at least a portion of a nucleotide sequence (or complement thereof) that is being screened for the presence of an associated genomic region, preferably at least about 80% identical, more preferably at least about 90% identical, even more preferably about 95% identical, or even 100% identical. In any embodiment, a probe or primer may be optionally labeled with, for example, a radioactive, fluorescent, biotinylated or chemiluminescent label (e.g., radioisotope, fluorescent compound, enzyme, or enzyme co-factor.) Labeled nucleic acids are useful for detection of a hybridization complex and can be used as probes for diagnostic and screening assays.

Labeled probes can be used in cloning of full-length cDNA or genomic DNA by screening cDNA or genomic DNA libraries. Classical methods of constructing cDNA libraries are taught in Sambrook et al., supra. These methods provide for the production of cDNA from mRNA and the insertion of the cDNA into viral or other expression vectors. Typically, libraries of mRNA comprising poly(A) tails can be produced with poly(T) primers. Similarly, cDNA libraries can be produced using the nucleic acids herein as primers. Libraries of cDNA can be made either from selected tissues (e.g., normal or diseased tissue), or from tissues of a mammal treated with, for example, a pharmaceutical agent. Alternatively, many cDNA libraries are available commercially. In a preferred embodiment, the cDNA library is made from diseased or healthy human neuronal tissues or cells. In another preferred embodiment, members of the cDNA library are larger than a nucleic acid hybridization probe, and preferably contain the whole cDNA native sequence.

Genomic DNA can be isolated in a manner similar to the isolation of full-length cDNA. Briefly, the nucleic acids herein, or fragments, derivatives or complements thereof, can be used to probe a library of genomic DNA. Preferably, a genomic DNA library is obtained from neuronal tissue or cells but this is not essential. Such libraries can be in vectors suitable for carrying large segments of a genome, such as P1 or YAC, as described in detail in Sambrook et al., 9.4-9.30. In addition, genomic sequences can be isolated from human BAC libraries, which are commercially available from Research Genetics, Inc., Huntsville, Ala., USA, for example. As an alternative, full-length cDNA, genomic DNA, or any nucleic acid, fragment, derivative or complement thereof, can be obtained by synthesis.

2. Antisense and RNAi

Antisense nucleic acids, or mimetics thereof that are complementary, in whole or in part, to one or more AD nucleic acids are provided. Antisense nucleic acids can be used in diagnostics, prognostics, theranostics and/or treatment of AD-related disease. Antisense nucleic acids hybridize under high stringency conditions to target nucleic acids (e.g., associated genomic regions or RNA derivatives thereof such as mRNA). An antisense nucleic acid can bind RNA to form a duplex or a double-stranded DNA to form a triplex, which may be assayed.

Preferably, hybridization of an antisense nucleic acid can act directly to block the translation of mRNA associated with susceptibility to AD-related disease by hybridizing to targeted mRNA and preventing protein translation. Absolute complementarity, although preferred, is not required. Antisense nucleic acids complementary to non-coding target nucleic acids associated with susceptibility to AD-related disease may also be used to inhibit translation of endogenous mRNA associated with susceptibility to AD-related disease by hybridizing to DNA regions involved in the transcription of the mRNA (e.g., regulatory regions, promoters, enhancers, etc.). While antisense nucleic acids complementary to a coding region sequence could be used, those complementary to the transcribed, untranslated region are most preferred. Antisense nucleic acids are preferably at least 10 nucleotides in length, more preferably at least 20 nucleotides, even more preferably at least 40 nucleotides in length, or more preferably at least 80 nucleotides in length. An antisense nucleic acid can be labeled for convenient detection, such as by using a radioisotope, fluorescent compound, enzyme or an enzyme co-factor.

Regardless of the choice of target sequence, it is preferred that in vitro studies be first performed to quantitate the ability of the antisense nucleic acid to inhibit mRNA expression. It is preferred that these in vitro studies utilize controls that distinguish between antisense inhibition and nonspecific biological effects of nucleic acids in a sample. Additionally, it is envisioned that results obtained using the antisense nucleic acid be compared with those obtained using a control nucleic acid. A control nucleic acid is preferably of approximately the same length as the test antisense nucleic acid and differs from the antisense nucleic acid sequence no more than is necessary to prevent specific hybridization to the target sequence.

The antisense nucleic acids herein can be modified at the base moiety, sugar moiety or phosphate backbone to improve stability of the molecule. Furthermore, the antisense nucleic acids may be hybridized or conjugated to another molecule (e.g., a peptide, hybridization triggered cross-linking agent, cleavage agent or transport agent) for targeting in a host cell or to facilitate the transport across the cell membrane (see, e.g., Letsinger et al. (1989) Proc. Natl. Acad. Sci. USA 86:6553-6556; Lemaitre et al., (1987), Proc. Natl. Acad. Sci. USA 84:648-652); for blood-brain barrier (see, e.g., PCT Publication No. WO89/10134); to facilitate the hybridization-triggered cleavage agents (see, e.g., Krol et al. (1988) BioTechniques 6:958-976) or intercalating agents (see, e.g., Zon, (1988), Pharm. Res. 5:539-549).

The antisense nucleic acids may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

The antisense nucleic acid may also comprise at least one modified sugar moiety selected from the group including but not limited to arabinose, 2-fluoroarabinose, xylulose, and hexose. In yet another embodiment, the antisense nucleic acid comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense nucleic acid is an α-anomeric oligonucleotide. An α-anomeric oligonucleotide forms specific double-stranded hybrids with complementary RNA in which, contrary to the usual β-units, the strands run parallel to each other (Gautier, et al., (1987) Nucl. Acids Res. 15:6625-6641). The oligonucleotide is a 2′-0-methylribonucleotide (Inoue, et al., (1987) Nucl. Acids Res. 15:6131-6148), or a chimeric RNA-DNA analogue (Inoue, et al., (1987) FEBS Lett. 215:327-330).

Antisense nucleic acids (as well as other nucleic acids) herein may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein, et al. (1988) Nucl. Acids Res. 16:3209, and methylphosphonate oligonucleotides can be prepared by use of controlled pore glass polymer supports Sarin, et al., (1988) Proc. Natl. Acad. Sci. USA 85:7448-7451, etc. Alternately, an antisense nucleic acid can be produced biologically by placing a target nucleic acid in an expression vector in an antisense orientation or by using reverse transcriptase along with other reagents to construct the complementary DNA stand.

Antisense nucleic acids should be delivered to cells that express the target nucleic acid in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies which specifically bind receptors or antigens expressed on the target cell surface) can be administered systemically.

A preferred approach to achieve intracellular concentrations of an antisense molecule sufficient to suppress translation of endogenous mRNAs utilizes a recombinant DNA construct in which the antisense oligonucleotide is placed under the control of a strong promoter (e.g., pol III or pol II). The use of such a construct to transfect target cells in a patient will result in the transcription of sufficient amounts of single stranded RNAs which will form complementary base pairs with the endogenous sequence transcripts and thereby prevent translation of the mRNA sequence. For example, a vector can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, (1981) Nature 290:304-310), the promoter contained in the 3′-long terminal repeat of Rous sarcoma virus (Yamamoto, et al., (1980) Cell 22:787-797), the herpes thymidine kinase promoter (Wagner, et al., (1981) Proc. Natl. Acad. Sci. USA. 78:1441-1445), and the regulatory sequences of the metallothionein gene (Brinster, et al., (1982) Nature 296:39-42). Any type of plasmid, cosmid, YAC or viral vector can be used to prepare the recombinant DNA construct that can be introduced directly into the tissue site. Alternatively, viral vectors can be used that selectively infect the desired tissue, in which case administration may be accomplished by another route (e.g., systemically).

In any of the embodiments herein, it may be necessary to compare the nucleotide sequence of the nucleic acid obtained, isolated, amplified, or cloned with that of a control. The percent identity of two nucleotide sequences can be determined, for example, by aligning the sequences for optimal comparison purposes. The nucleotides at corresponding positions are compared and the percent identity between the two sequences is a function of the number of identical positions shared by the sequences (e.g., percent identity=[(the number of identical positions/total number of positions)×100]. In some embodiments, the length of a sequence aligned for comparison purposes is at least 30%, preferably at least 40%, more preferably at least 60%, and even more preferably at least 70%, 80%, or 90% of the length of the reference sequence or a full sequence gene. An actual comparison of two nucleic acid sequences can be accomplished by well-known methods, for example, using a mathematical algorithm. In one example, such a mathematical algorithm is described in Karlin et al., (1993) Proc. Natl. Acad. Sci. USA, 90:5873-5877. In another example, such mathematical algorithm is the algorithm of Myers and Miller, (1989) CABIOS. Additional algorithms for sequence analysis are known in the art and include ADVANCE and ADAM as described in Torellis and Robotti (1994) Comput. Appl. Biosci., 10:3-5 and FASTA described in Pearson and Lipman (1988) Proc. Natl. Acad. Sci. USA, 85:2444-8.

RNAi, or “RNA interference” is a technique in which exogenous, double-stranded RNA complementary to a known target mRNA are introduced into a cell to cause the degradation of the target mRNA, thereby reducing or silencing gene expression. This method of gene regulation has been demonstrated in Drosophila, Coenorhabditis elegans, plants, and in mammalian cell cultures. In mammalian cells, siRNAs (“small-interfering RNAs” that are double-stranded) are transfected into cells. siRNAs can be created using a phage enzyme known as “DICER” and a multi-protein siRNA complex termed “RISC” (RNA induced silencing complex). Briefly, duplexes of short (˜19 nucleotides in length) RNAs with symmetric 2-nucleotide 3′-overhangs (siRNAs) are introduced into a cell where they associate with specific proteins in a ribonucleoprotein complex, which scans the mRNA in the cell and degrades the mRNA target that is homologous to the siRNA, thereby preventing translation of the mRNA message and, therefore, synthesis of the protein encoded therein. For a review of RNAi techniques, see, e.g., Huppi, et al. (2005) “Defining and Assaying RNAi in Mammalian Cells”, Molecular Cell 17(1):1-10; Grimm, et al. (2005) “Adeno-associated virus vectors for short hairpin RNA expression”, Methods Enzymol 392:381-405; Bantounas, et al. (2004) “RNA interference and the use of small interfering RNA to study gene function in mammalian systems”, J Molec Endocrin 33:545-557; Genc, et al. (2004) “RNA interference in neuroscience”, Brain Res Mol Brain Res 132(2):260-270; and Campbell, et al. (2005) “RNA interference: past, present and future”, Curr Issues Mol Biol 7(1):1-6.

3. Ribozymes, Knock-Outs and Triple Helices

Ribozyme molecules designed to catalytically cleave target mRNA transcripts can also be used to prevent translation of such mRNA. See, e.g., WO 90/11364; Sarver, et al., (1990) Science 247: 1222-1225.

Ribozymes are enzymatic RNA molecules capable of catalyzing the specific cleavage of RNA. See Rossi, (1994) Current Biology 4:469-471. The mechanism of ribozyme action involves sequence specific hybridization of the ribozyme to complementary target RNA, followed by an endonucleolytic cleavage event. The composition of ribozyme molecules must have one or more sequences complementary to the target mRNA and must include the well known catalytic sequence responsible for mRNA cleavage. See, e.g., U.S. Pat. No. 5,093,246.

While ribozymes that cleave mRNA at site-specific recognition sequences can be used to destroy target mRNAs, the use of hammerhead ribozymes is preferred. Hammerhead ribozymes cleave mRNAs at locations dictated by flanking regions which form complementary base pairs with the target mRNA. The sole requirement is that the target mRNA have the following sequence of two bases: 5′-UG-3′. The construction and production of hammerhead ribozymes are well known in the art and are described in Myers, “Molecular Biology and Biotechnology: A Comprehensive Desk Reference,” (VCH Publishers, New York, 1995) page 833; and in Haseloff and Gerlach, (1988), Nature 334:585-591.

Preferably a ribozyme is engineered so that the cleavage recognition site is located near the 5-end of the target mRNA, i.e., to increase efficiency and minimize the intracellular accumulation of non-functional mRNA transcripts.

The ribozymes herein may further include RNA endoribonucleases, also known as “Cech-type ribozymes,” such as the one which occurs naturally in Tetrahymena thermophila (known as the IVS, or L-19 IVS RNA) and which has been extensively described in Zaug, et al., (1984) Science 224:574-578; Zaug and Cech, (1986) Science 231:470-475; Zaug, et al., (1986) Nature 324:429-433; PCT Publication No. WO 88/04300; Been and Cech, (1986) Cell 47:207-216.

As in the antisense approach, ribozymes can be composed of modified nucleic acids (e.g., for improved stability, targeting, etc.) and are preferably delivered to cells that express the target gene in vivo. A preferred method of delivery involves using a DNA construct encoding the ribozyme under the control of a strong constitutive promoter (e.g., pol III or pol II), so that transfected cells will produce sufficient quantities of the ribozyme to destroy endogenous target mRNA and inhibit translation. Because ribozymes, unlike antisense molecules, are catalytic, a lower intracellular concentration is required for efficiency.

Endogenous target gene expression can also be reduced by inactivating or “knocking out” the target nucleic acid (e.g., coding regions or regulatory regions of the target gene) using targeted homologous recombination. See Smithies, et al., (1985) Nature 317:230-234; Thomas and Capecchi, (1987) Cell 51:503-512; Thompson, et al., (1989) Cell 5:313-321. For example, a non-functional nucleic acid (or a completely unrelated DNA sequence) flanked by DNA homologous to the endogenous target nucleic acid can be used, with or without a selectable marker and/or a negative selectable marker, to transfect cells which express the target gene in vivo. Insertion of the DNA construct, via targeted homologous recombination, results in inactivation of the target gene. Such approaches can be used in humans provided the recombinant DNA constructs are directly administered or targeted to the required site in vivo using appropriate viral vectors.

Alternatively, endogenous expression of a target gene can be reduced by targeting deoxyribonucleotide sequences complementary to the regulatory region of the target gene (i.e., the target gene promoter and/or enhancers) to form triple helical structures which prevent transcription of the target gene in target cells in the body. See generally, Helene, (1991), Anticancer Drug Des., 6(6):569-584; Helene, et al., (1992), Ann. N.Y. Acad. Sci., 60:27-36; and Maher, (1992), Bioassays 14(12):807-815.

Nucleic acids to be used in triple helix formation for the inhibition of transcription should be single-stranded and composed of deoxyribonucleotides. The base composition of these oligonucleotides must be designed to promote triple helix formation via Hoogsteen base pairing rules, which generally require sizable stretches of either purines or pyrimidines to be present on one strand of a duplex. Nucleic acids may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen which are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so-called “switchback” nucleic acid. Switchback nucleic acids are synthesized in an alternating 5′-3′, 3′-5′ manner, such that they base pair with first one strand of a duplex and then the other, eliminating the necessity for a sizable stretch of either purines or pyrimidines to be present on one strand of a duplex.

In instances wherein the antisense, ribozyme, “knock-out,” and/or triple helix molecules described herein are utilized to inhibit gene expression (e.g., expression of nucleic acids associated with susceptibility to AD-related disease), it is possible that the technique may so efficiently reduce or inhibit the transcription (triple helix; knock-out) and/or translation (antisense, ribozyme) of mRNA that it may cause severe negative side effects. In such cases, to ensure that substantially normal levels of target gene products or desired gene products are maintained, nucleic acids which encode and polypeptides exhibiting a desired target gene activity (e.g., polypeptides associated with resistance to AD-related disease) may, be introduced into cells via gene therapy methods. The desired gene product should not contain sequences susceptible to antisense, ribozyme or triple helix treatments that are being utilized.

The antisense, ribozyme and triple helix molecules herein may be prepared by any method known in the art for the synthesis of DNA and RNA molecules.

4. Expression Vectors and Vectors

In certain embodiments, the nucleic acids herein are used to over-express polypeptides associated with resistance to AD-related disease. In another embodiment, the nucleic acids herein are used to underexpress polypeptides associated with susceptibility to AD-related disease. To overexpress a polypeptide, for example, a nucleic acid encoding the polypeptide of interest can be ligated to a regulatory sequence that can drive the expression of the polypeptide in the animal cell type of interest at a level that is higher than expression in the absence of such a construct. Such regulatory regions are well known. In another example, a non-coding nucleic acid (e.g., an intron or a regulatory nucleic acid) may be introduced to increase the production of a polypeptide of interest. To underexpress an endogenous polypeptide, a nucleic acid encoding a transcription factor or antisense RNA that down-regulates the polypeptide or a nucleic acid that produces, e.g., a variant or inactive polypeptide may be introduced into the genome of an animal such that the endogenous expression will be reduced or inactivated. In addition to, or in the alternative, a non-coding nucleic acid herein (e.g., an intron or a regulatory nucleic acid) may be introduced to override a native regulatory nucleic acid.

Any one or more of the nucleic acids herein can be inserted into a vector. A vector can be used, for example, to transfer nucleic acids or to express the inserted nucleic acids. In one embodiment, nucleic acids comprising an exon associated gene region of lu, pvrl2, tomm40, apoE, apoC1, apoC2, apoC4, clptm1 or a homolog or fragment thereof can be inserted into an expression vector to express a partial or complete AD gene product. In another embodiments an exon associated gene region of a2bp1, ahsg, apoE, app, c9orf52, cacna1c, ckm, ctnnd2, cugbp1, dkfzp566k1924, fars1, fgl2, flj14442, flj36760, flj38736, kiaa1486, kiaa1862, laptm4a, lnx2, loc147468, loc166522, loc283867, loc387711, loc388110, loc401237, lrp1B, matn3, mgc3971, mrlc2, nce2, pcbp3, pde11A, pfkfb2, ppp1r12b, psen1, pvrl2, sec13L1, sox5, tgds, tomm40, ttll2 or homologs or fragments thereof can be inserted into an expression vector to express a partial or complete AD gene product.

An exonic associated genomic region can be in the coding region or outside the coding region. Expression vectors may be constructed using methods known in the art. Such methods include in vitro recombinant DNA techniques, synthetic techniques, in vivo genetic recombination, and other techniques described in Sambrook, J. et al. “Molecular Cloning, A Laboratory Manual,” (Cold Spring Harbor Press, Plainview, N.Y. 1989), and Ausubel, F. M. et al. “Current Protocols in Molecular Biology”, (John Wiley & Sons, New York, N.Y., 1989). A vector may also comprise one or more regulatory elements that direct the expression of a coding sequence in a host cell. Regulatory elements include but are not limited to inducible and non-inducible promoters, enhancers, operators, and other elements that drive and regulate expression.

There are numerous types of expression vectors. One type of expression vector is a plasmid, which refers to a circular double stranded DNA molecule into which additional DNA segments can be ligated. Another type of vector is a viral vector, wherein additional DNA segments can be ligated into a viral genome. Viral vectors include replication defective retroviruses, adenoviruses and adeno associated viruses. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, e.g., expression vectors, are capable of directing the expression of genes to which they are operably linked. A preferable expression vector is a plasmid, an artificial chromosome, a cosmid or a viral vector.

The expression vectors herein can include one or more regulatory sequences, selected on the basis of the host cells to be used and the level of expression desired. The regulatory sequences can be operably linked to the nucleic acid sequence to be expressed. The term operably linked refers to a nucleic acid of interest that is linked to one or more regulatory sequences in a manner that allows for the expression of the nucleic acid of interest. The term regulatory sequence includes promoters, enhancers and other expression control elements (e.g., polyadenylation signals). Such regulatory sequences are described, for example, in Goeddel, “Gene Expression Technology: Methods in Enzymology” (1990) 185, Academic Press, San Diego, Calif. Regulatory sequences include those that direct constitutive expression of a nucleotide sequence in many types of host cells and those that direct expression of the nucleotide sequence only in certain host cells (e.g., tissue-specific regulatory sequences).

In another embodiment, a coding region of an associated genomic region can be inserted into an expression vector with or without a non-coding region of interest. The difference in expression or activity between a vector comprising both the non-coding and coding sequence can be detected using methods known in the art.

The vectors herein can be inserted into a host cell. The term “host cell” refers not only to a particular subject cell but also to the progeny or potential progeny of such a cell. Because certain modifications may occur in succeeding generations due to either mutations or environmental influences, such progeny may not, in fact, be identical to cells, but are still included within the scope of the term as used herein.

Vectors can be introduced into prokaryotic or eukaryotic cells via conventional transformation or transfection techniques. For example, expression systems in bacteria include those described in Chang et al., (1978) Nature 275:615, and Siebenlist et al., (1980) Cell 20:269; expression systems in yeast include those described in Kelly and Hynes, EMBO J. (1985) 4:475-479; expression systems in insect cells include those described in Maeda et al., (1985) Nature 315:592-594 and expression in mammalian cells include those described, for example, in Dijkema et al., (1985) EMBO J. 4:761. Vector constructs can comprise either sense or antisense sequences, or both.

As used herein, the terms transformation and transfection refer to a variety of art-recognized techniques for introducing a foreign nucleic acid molecule (e.g., DNA) into a host cell, including calcium phosphate or calcium chloride co-precipitation, DEAF-dextran-mediated transfection, lipofection, or electroporation. Suitable methods for transforming or transfecting host cells can be found in Sambrook, et al. and other laboratory manuals. For stable transfection of mammalian cells, it is known that, depending upon the expression vector and transfection technique used, only a small fraction of cells may integrate the foreign DNA into their genome. To identify and select these integrants, a gene that encodes a selectable marker is generally introduced into the host cells along with the gene of interest. Preferred selectable markers include those that confer resistance to drugs. Nucleic acid molecules encoding a selectable marker can be introduced into a host cell on the same vector as the nucleic acids or can be introduced on a separate vector. Cells stably transfected with the introduced nucleic acid molecule can be identified by drug selection (e.g., cells that have incorporated the selectable marker gene will survive, while the other cells die).

A variety of host-expression vector systems may be utilized to express the AD coding nucleic acids of the invention. Such host expression systems represent not only the vectors by which the coding sequences may be expressed and their encoded RNAs or polypeptides purified, but also represent the cells containing these vectors. These include, but are not limited to bacteria (e.g., E. coli, B. subtilis) transformed with recombinant bacteriophage DNA, plasmid DNA or cosmid DNA expression vectors; yeast (e.g., Saccharomyces, Pichia) transformed with recombinant yeast expression vectors, insect cell systems transformed with recombinant viral expression vectors (e.g., baculovirus), plant cell systems transformed with recombinant viral expression vectors (e.g., cauliflower mosaic virus, tobacco mosaic virus) or transformed with recombinant plasmid expression vectors (e.g., Ti plasmid), and mammalian cell systems (e.g., COS, CHO, BHK, 293, 3T3 cell lines) transformed with recombinant expression constructs containing promoters derived from the genome of mammalian cells (e.g., metallothionein promoter) or from mammalian viruses (e.g., the adenovirus late promoter, vaccinia virus 7.5K promoter). Such vectors and host-expression vector systems are well known in the art and are further described in, e.g., Ruther et al. (1983), EMBO J. 2:1791; Inouye & Inouye (1985) Nucleic Acids Res. 13:3101-3109; Van Heeke & Schuster (1989) J. Biol. Chem. 264:5503-5509; Smith et al. (1983) J. Virol. 46:584; Smith, U.S. Pat. No. 4,215,051; Logan & Shenk (1984) Proc. Natl. Acad. Sci. USA 81:3655-3659; Bittner et al. (1987) Methods in Enzymol. 153:516-544; Alam (1990) Anal. Biochem. 188:245-254; MacGregor & Caskey (1989) Nucl. Acids Res. 17:2365; and Norton & Corrin (1985) Mol. Cell. Biol. 5: 281.

In addition, a host cell may be chosen that modulates the expression of a vector-encoded nucleic acid sequence, or that modifies and processes an encoded RNA or polypeptide in a specific manner. Such modifications (e.g., glycosylation, phosphorylation) and processing (e.g., cleavage, folding) of polypeptides may be important for the function of the polypeptide. Different host cells have characteristic and specific mechanisms for the post-translational modification of polypeptides. Appropriate host cells or systems can be chosen to ensure that the correct modification and processing of an encoded protein. As such, in some situations, it may be desirable to express a eukaryotic gene in a eukaryotic cell where the gene will benefit from native folding and posttranslational modifications. Such mammalian host cells include, but are not limited to CHO, VERO, BHK, HeLa, COS, MDCK, 293, 3T3, W138, etc.

Host cells can be used to produce polypeptides encoded by any of the nucleic acids herein. Suitable host cells and methods for producing polypeptides using such host cells are discussed in Goeddel, supra. For large scale protein production, a unicellular organism such as E. coli, baculovirus vectors, or cells of higher organisms such as vertebrates, particularly mammals, e.g. COS7 cells, may be useful. Host cells into which an expression vector has been introduced may be cultured in suitable medium such that the polypeptide is produced. The polypeptide herein may be isolated from the medium or from the host cell.

Host cells can also be used to produce nonhuman transgenic animals. For example, in one embodiment, a host cell is a fertilized oocyte or an embryonic stem cell into which a nucleic acid (e.g., an exogenous AD gene or a nucleic acid encoding a polypeptide herein) has been introduced. Such host cells can then be used to create non-human transgenic animals in which exogenous nucleotide sequences have been introduced into the genome or homologous recombinant animals in which endogenous nucleotide sequences have been altered. Such animals are useful for studying the function and/or activity of the nucleotide sequence and polypeptide encoded by the sequence and for identifying and/or evaluating modulators of their activity. As used herein, a “transgenic animal” is a non-human animal, preferably a mammal, more preferably a rodent such as a rat or mouse, in which one or more of the cells of the animal include a transgene. Other examples of transgenic animals include, for example, non-human primates, sheep, dogs, cows, goats, chickens and amphibians. A transgene is an exogenous DNA which is integrated into the genome of a cell from which a transgenic animal develops and which remains in the genome of the mature animal, thereby directing the expression of an encoded gene product in one or more cell types or tissues of the transgenic animal. As used herein, an homologous recombinant animal is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

Methods for generating transgenic animals via embryo manipulation and microinjection, particularly animals such as mice, are conventional in the art and are described, for example, in U.S. Pat. Nos. 4,736,866, 4,870,009, 4,873,191 and in Hogan, “Manipulating the Mouse Embryo,” (Cold Spring Harbor Laboratory Press, Cold Spring Harbor, N.Y., 1986). Methods for constructing homologous recombination vectors and homologous recombinant animals are described further in Bradley (1991) Current Opinion in BioTechnology, 2:823-829. Clones of the non-human transgenic animals described herein can also be produced according to the methods described in Wilmut et al. (1997) Nature 385:810-813; and PCT Publication Nos. WO 97/07668 and WO 97/07669.

III Polypeptides

AD polypeptides such as those encoded by or regulated by associated genomic regions comprising the variants, preferably, the susceptibility variants, identified in FIG. 1, 2 or 3. For example, AD polypeptides include those encoded by a TOMM40 exon encompassing a “BEST_POSITION” of 50087984 or any fragment, complement, derivative, homolog, or analog thereof. In other embodiments, an AD polypeptide is one that is regulated by an intronic TOMM40 region that encompasses a “BEST_POSITION” of 50095698 and/or 50096271. In other embodiments, an AD polypeptide herein is regulated by an intron of APOE that encompasses a “BEST_POSITION” of 50102284. In other embodiments, an AD polypeptide herein is regulated by an intron of PVRL2 that encompasses a “BEST_POSITION” of 50053064.

The AD polypeptides herein may be naturally occurring or recombinantly produced using methods known in the art.

An AD polypeptide can be associated with resistance or susceptibility to AD. A polypeptide associated with resistance to AD may be one that is expressed differently in individuals having a phenotype of resistance to AD as compared to individuals who do not have a phenotype of resistance to AD, or one that is regulated or encoded in whole or in part by a nucleic acid associated with resistance to AD. In one example, a polypeptide associated with AD can be recombinantly produced using an expression vector having a non-coding regulatory region associated with resistance to AD, operably linked to an AD polypeptide. The expression vector is introduced into a host cell under conditions appropriate for expression. The polypeptide can then be isolated from the host cell using standard protein purification techniques.

Similarly, a polypeptide associated with susceptibility to AD may be one that is expressed differently in individuals having a phenotype of susceptibility to AD as compared to individuals who do not have a phenotype of susceptibility to AD, or one that is regulated or encoded, in whole or in part, by nucleic acids associated with susceptibility to AD. For example, a polypeptide associated with AD can be recombinantly produced by introducing an expression vector with a coding nucleic acid associated with susceptibility to AD into a host cell. The host cell is maintained under conditions suitable for expression. The polypeptide is then isolated from the host cell.

In one embodiment, a polypeptide associated with resistance to AD can be produced by inserting a non-coding nucleic acid or nucleic acid outside coding region which is associated with resistance to AD, operably linked to an associated genomic region coding sequence, into a host cell under conditions appropriate for protein synthesis, and then purifying the polypeptide expressed by the host cell

A similar method can be used to produce a polypeptide associated with susceptibility to AD. For example, a non-coding nucleic acid or nucleic acid outside coding region which is associated with susceptibility to AD, operably linked to an associated genomic region coding sequence, can be inserted into a host cell under conditions appropriate for protein synthesis. The resulting polypeptide associated with susceptibility to AD is then collected and purified.

In a preferred embodiment, a polypeptide associated with susceptibility to AD can be produced by inserting a vector comprising a coding nucleic acid associated with susceptibility or resistance to AD and then purifying the polypeptide expressed by the host cell.

In preferred embodiments, the polypeptides are purified. There are various degrees of purity. While a polypeptide can be purified to homogeneity, preparations in which a polypeptide is not purified to homogeneity are also useful where the polypeptide retains a desired function even in the presence of considerable amount of other components. In some embodiments, polypeptides are substantially free of cellular material which includes preparations of a polypeptide having less than about 30% (dry weight) other polypeptides (e.g., contaminating polypeptides), less than about 20% other polypeptides, less than about 10% other polypeptides, or less than about 5% other polypeptides.

When a polypeptide is recombinantly produced, it can also be substantially free of culture medium. In preferred embodiments, culture medium represents less than about 20% of the volume of the polypeptide preparation, preferably less than about 10% of the volume of the polypeptide preparation or more preferably less than about 5% of the volume of the polypeptide preparation. Polypeptides that are substantially free of chemical precursors or other chemicals generally include those that are separated from chemicals that are involved in its synthesis. In one embodiment, the polypeptides are substantially free of chemical precursors or other chemicals such that a preparation of the polypeptides has less than about 30% (dry weight) chemical precursors or other chemicals, preferably less than about 20% chemical precursors or other chemicals, more preferably less than about 10% chemical precursors or other chemicals or more preferably less than about 5% chemical precursors or other chemicals.

As used herein, two polypeptides are substantially homologous when their amino acid sequences are at least about 45% homologous, or preferably at least about 75% homologous, or more preferably at least about 85% homologous, or even more preferably greater than about 95% homologous. To determine the percent homology of two polypeptides, the amino acid sequences are aligned for optimal comparison purposes. The amino acid residues at corresponding positions are compared. The percent homology between two amino acid sequences is a function of the number of identical positions shared by the sequences (e.g., percent homology equals the number of identical positions/total number of positions times 100).

Some polypeptides (e.g., synonymous or conservative variants) may have a lower degree of sequence homology but are still able to perform one or more of the same functions. Conservative substitutions that can maintain the same function include replacements among aliphatic amino acids methionine, valine, leucine and isoleucine; interchange of the hydroxyl residues serine and threonine; exchange of acidic residues aspartic and glutamic acids; substitution between amide residues asparagine and glutamine, exchange between basic residues lysine and arginine, and replacements among aromatic residues phenylalanin, tyrosine and tryptophan. Alanine and glycine may also result in conservative substitutions.

Other polypeptides that may not be able to perform one or more of the same functions may be variants containing one or more non-conservative amino acid substitutions or deletions, insertions, inversions or substitution of one or more amino acid residues. Amino acids that are essential for function of a polypeptide can be identified by various methods known in the art, such as site-directed mutagenesis or alanine-scanning mutagenesis. See Cunningham et al., (1989) Science, 244:1081-1085. The latter procedure can introduce a single alanine mutation at every residue in the molecule. The resulting variants are then tested for biological activity in vitro or in vivo. Residues that are critical for polypeptide activity or inactivity are identified by comparing the two variants (with and without the alanine mutation). Polypeptide activity can also be determined by structural analysis such as crystallization, nuclear magnetic resonance or photoaffinity labeling. See Smith et al, (1992) J. Mol. Biol., 224:899-904; and de Vos et al. (1992) Science, 255:306-312.

1. Fusion Proteins

Any polypeptides herein can be made part of a fusion protein. The term “fusion protein” or “fusion polypeptide” refers to an AD polypeptide (a polypeptide associated with resistance or susceptibility to AD) operatively linked to a non-AD polypeptide or a heterologous polypeptide having an amino acid sequence not substantially homologous to an AD amino acid sequence. “Operatively linked” indicates that the polypeptide and the heterologous protein are fused, for example, the non-AD polypeptide can be fused to the N-terminus or C-terminus of the AD polypeptide. In a preferred embodiment, the fusion polypeptide does not affect the function of the AD polypeptide. Examples of fusion polypeptide that do not affect the function of a polypeptide include a GST-fusion polypeptides in which the AD polypeptide sequences are fused to the C-terminus of the GST sequences. Other types of fusion polypeptides include enzymatic fusion polypeptides, for example β-galactosidase fusions, yeast two-hybrid GAL fusions, poly-His fusions and Ig fusions. Fusion polypeptides, especially poly-His fusions, can facilitate the purification of recombinant polypeptide. In some host cells, such as mammalian cells, expression and secretion of an AD polypeptide can be increased using a heterologous signal sequence. Therefore, in a preferred embodiment, an AD polypeptide may be fused to a heterologous signal sequence at its N-terminus. In another embodiment, a fusion protein may comprise of an AD polypeptide and various portions of immunoglobulin constant regions such as the Fc portion. Fc portions are useful in therapy and diagnosis and may result in improved pharmacokinetic properties. Fc portions can also be used in high-throughput screening assays to identify binding molecules, agonists and antagonists. See, e.g., Bennett et al.; J. of Molec. Recog., (1995) 8:52-58 and Johanson et al., (1995) J. of Biol. Chem., 270,16:9459-9471. In a preferred embodiment, soluble fusion proteins comprise of an AD polypeptide and one or more of the constant regions of heavy or light chains of immunoglobulins (e.g. IgG, IgM, IgA, IgD, IgE).

A fusion protein can be produced by standard recombinant DNA techniques as described herein. For example, DNA fragments coding for the different polypeptide sequences are ligated together in accordance with conventional techniques. The fusion gene can be synthesized by conventional techniques such as automated DNA synthesizers. Alternatively, PCR amplification of nucleic acid fragments can be carried out using anchor primers which give rise to complementary overhangs between two consecutive nucleic acid fragments that can subsequently be annealed and reamplified to generate a chimeric nucleic acid sequence. Moreover, many expression vectors are commercially available that already encode a fusion moiety (e.g., a GST protein). A nucleic acid encoding a polypeptide herein can be cloned into such an expression vector such that the fusion moiety is linked in-frame to the polypeptide.

2. Antibodies

Any of the polypeptides herein, or fragments, derivatives, or complements thereof, can be used as an immunogen (e.g. epitope) to generate polypeptide-specific antibodies. Antibodies can be used to detect, isolate and inhibit the activity of one or more AD polypeptides.

To generate AD antibodies, an AD polypeptide or a fragment thereof is used as an epitope. In preferred embodiments, an epitope is at least 6 amino acids, at least 9 amino acids, at least 20 amino acids, at least 40 amino acids, or at least 80 amino acids in length. The epitope or polypeptide fragment preferably comprises a domain, segment or motif that can be identified by analysis using well-known methods, for example, signal polypeptides, extracellular domains, transmembrane segments or loops, ligand binding regions, zinc finger domains, DNA binding domains, acylation sites, glycosylation sites or phosphorylation sites.

Examples of antibodies contemplated by the present invention include polyclonal, monoclonal, humanized, chimeric, single chain antibodies, antibody fragments such as Fab fragments, F(ab′)2 fragments, fragments produced by Fab expression library, anti-idiotypic (anti-Id) antibodies and epitope-binding fragments of any of the above.

Polyclonal antibodies are prepared by immunizing a suitable subject (e.g., goats, rabbits, rats, mice or humans) with a desired antigen. The antibody titer in the immunized subject can be monitored over time using methods known in the art, such as by using an enzyme linked immunosorbent assay (ELISA). The antibodies can then be isolated from the subject (e.g., from blood) and further purified using techniques, such as protein A chromatography, to obtain the IgG fraction.

At an appropriate time after immunization, such as when the antibody titers are highest, antibody-producing cells can be obtained from the subject and used for the preparation of monoclonal antibodies. Monoclonal antibodies are populations of antibodies that contain only one species of an antigen-binding site and are capable of immunoreacting with only one particular epitope of AD polypeptides. A monoclonal antibody composition, therefore, typically displays a single binding affinity for a particular polypeptide with which it immunoreacts.

There are numerous methods known in the art for producing monoclonal antibodies. In one example, monoclonal antibodies can be obtained by fusing individual lymphocytes (typically splenocytes) from an immunized animal (typically a mouse or a rat) with cells derived from an immortal B lymphocyte tumor (typically a myeloma) to produce a hybridoma. The culture supernatants of the resulting hybridoma cells are screened to identify a hybridoma producing a monoclonal antibody that specifically binds to a polypeptide of interest. Other techniques for producing hybridoma include the human B cell hybridoma technique described in Kozbor et al. (1983) Immunol. Today, 4:72; the EBV-hybridoma technique and the trioma techniques.

Alternatively, monoclonal antibodies can be identified and isolated by screening a combinatorial immunoglobulin library, such as an antibody phage display library. The library can be screened with one or more of the polypeptides herein. Identified members are then isolated using techniques known in the art. Kits for generating and screening phage display libraries are commercially available. See for example, the Pharmacia Recombinant Phage Antibody System, Catalog No. 27-9400-01, and the Stratagene SurjZAPTM Phage Display Kit, Catalog No. 240612. Other methods and reagents for generating and screening antibody display libraries are disclosed in PCT Publication No. WO 92/01047; PCT Publication No. WO 90/02809; Fuchs et al. (1991) Bio/Technology, 9:1370-1372; Hay et al. (1992) Hum. Antibod. Hybridomas, 3:81-85; Huse et al. (1989) Science 246:1275-1281; Griffith et al. (1993) EMBO J. 12:725-734.

The monoclonal antibodies can be chimeric and humanized. Humanized monoclonal antibodies can be obtained using standard recombinant DNA techniques in which the variable region genes (e.g., of a rodent antibody), are cloned into a mammalian expression vector containing the appropriate human light change and heavy chain region genes. In this example, the resulting chimeric monoclonal antibodies has the antigen-binding capacity from the variable region of the rodent but is significantly less immunogenic because of the humanized light and heavy chain regions. See, e.g., Surender K. Vaswani, Ann. (1998) Allergy Asthma. Immunol. 81:105-119.

Any of the antibodies can further be coupled to a substance (label) for detection of a polypeptide-antibody binding complex. Examples of labels include, enzymes, prosthetic groups, fluorescent materials, luminescent materials, bioluminescent materials, or radioactive materials. Examples of suitable enzymes include, for example, horseradish peroxidase, alkaline phosphatase, β-galactosidase, or acetylcholinesterase. Examples of suitable prosthetic group complexes include, for example, streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin. An example of a luminescent material is luminol. Examples of bioluminescent materials include luciferase, luciferin and aequorin. Examples of suitable radioactive material include 1251, 1311, 35S or 3H.

The antibodies can be used to isolate one or more AD polypeptides using standard techniques such as affinity chromatography or immunoprecipitation. The antibodies can also be used to detect the presence or absence of a particular polypeptide (e.g., a polypeptide associated with resistance or susceptibility to AD) in a cell, cell lysate, cell supernatant, tissue sample or elsewhere. Preferably, the antibodies can further be used to inhibit or suppress the activity of such polypeptides by specifically binding to the polypeptides.

Some antibodies of the invention specifically bind to one variant form a protein encoded by a selected from the group consisting of: APOE, APOC, APP, C9orf52, CTNND2, CUGBP1, DKFZP566K1924, FARS1, FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC388110, MGC39715, NCE2, PFKFB2, PPP1R12B, PSEN1, TGD5, and TTLL2, without specifically binding to another. That is, antibodies specifically bind to a variant form having an amino acid encoded by a codon including a resistance allele shown in FIG. 3 or Table 11 without binding to a variant form having an amino acid encoded by a codon including a susceptibility allele shown in FIG. 3 or Table 11, (or vice versa). Such antibodies are useful, for example, in assays described below to detect variant forms of the above proteins.

IV Diagnostic And Prognostic Assays

The nucleic acids, polypeptides, antibodies and other compositions herein may be utilized as reagents (e.g., in pre-packaged kits) for prognosis and diagnosis of susceptibility or resistance to AD, and in particular LOAD. The methods can be practiced on subjects known to have one or more symptoms of an Alzheimer's related disease, such as dementia, as part of a differential diagnosis or prognosis of other diseases. The methods can also be practiced on subjects having a known susceptibility to an Alzheimer's disease. The polymorphic profile of such an individual can increase or decrease the assessment of susceptibility. For example, an individual having two siblings with Alzheimer's disease is known to be at increased susceptibility to the disease compared with the general population. A finding of additional factors favoring susceptibility increases the risk whereas finding factor favoring resistance decreases the risk.

The invention provides methods of determining the polymorphic profiling of an individual at one or more of SNPs of the invention. The SNPs includes those shown in FIG. 3 and Table 11, and those in linkage disequilibrium with them. Those in linkage disequilibrium with them usually occur in the same genes or within 10 or 40 kb of the same genes. SNPs in linkage disequilibrium with the SNPs in FIG. 3 or Table 11 can be determined by haplotype mapping. Haplotypes can be determined by fusing diploid cells from different species. The resulting cells are partially haploid, allowing determination of haplotypes on haploid chromosomes (see US 20030099964). Alternatively, SNPs in linkage disequilibrium with exemplified SNPs can be determined by similar association studies to those described in the examples below.

The polymorphic profile means the polymorphic forms occupying the various polymorphic sites in an individual. In a diploid genome, two polymorphic forms, the same or different from each other, usually occupy each polymorphic site. Thus, the polymorphic profile at sites X and Y can be represented in the form X(x1, x1), and Y (y1, y2), wherein x1, x1 represents two copies of allele x1 occupying site X and y1, y2 represent heterozygous alleles occupying site Y.

The polymorphic profile of an individual can be scored by comparison with the polymorphic forms associated with resistance or susceptibility to Alzheimer's disease occurring at each site as shown in FIG. 3. The comparison can be performed on at least 1, 2, 5, 10, 25, 50, or all of the polymorphic sites, and optionally, others in linkage disequilibrium with them. The polymorphic sites can be analyzed in combination with other polymorphic sites. However, the total number of polymorphic sites analyzed is usually fewer than 10,000, 1000, 100, 50 or 25.

The polymorphic profile is preferably determined at one or more polymorphic sites in each of at least 2, 5, 10, 15 or 20 of the following genes: APOE, APP, APOC1, C9orf52, CTNND2, CUGBP1, DKFZP566K1924, FARS1, FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC388110, MGC39715, NCE2, PFKFB2, PPP1R12B, PSEN1, TGD5, and TTLL2. In some methods, the polymorphic profile includes at least one site in at least 2, 5, 10, 15 or 20 sites in each of he following genes: APOE, APP, APOC1, CACNA1C, CKM, FARS1, KIAA1486, LOC147468, LOC166522, MGC39715, NCE2, PCBP3, PDE11A, PFKFB2, PVRL2, SEC13L1, TOMM40 and TTLL2. In some methods, the polymorphic profile includes at least 2, 5, 10, 15 or 20 sites in each of the following genes: APOC1, CACNA1C, CKM, FARS1, KIAA1486, LOC147468, LOC166522, MGC39715, NCE2, PCBP3, PDE11A, PFKFB2, PVRL2, SEC13L1, TOMM40 and TTLL2. Some methods determine a polymorphic profile in at least one site not within the APOE gene. Some methods determine a polymorphic profile in at least one polymorphic site not within any of the APP, PSEN1 or APOE genes. Some methods determine a polymorphic profile in at least one polymorphic site not within any of the APP, PSEN1, APOE, TOMM40 or APOC1 genes. Some method determine a polymorphic profile in at least one polymorphic site that does not occur within 40 kb of APP, PSEN1, APOE, TOMM40 or APOC1. Some methods detect susceptibility alleles at polymorphic sites identified by SNP ID's 42182, 42256, 42263 and/or 201401 in APP and PSEN1, or SNPs in linkage disequilibrium with any of these. Presence of one or more such susceptibility alleles is an indication of presence or susceptibility to LOAD.

The number of resistance or susceptibility alleles present in a particular individual can be combined additively or as ratio to provide an overall score for the individual's genetic propensity to Alzheimer's and related diseases (see U.S. Ser. No. 60,566,302, filed Apr. 28, 2004, U.S. Ser. No. 60/590,534, filed Jul. 22, 2004, U.S. Ser. No. 10/956,224 filed Sep. 30, 2004, and PCT US05/07375 filed Mar. 3, 2005. Resistance alleles can be arbitrarily each scored as +1 and susceptibility alleles as −1 (or vice versa). For example, if an individual is typed at 100 polymorphic sites of the invention and is homozygous for resistance at all of them, he could be assigned a score of 100% genetic propensity to resistance to Alzheimer's disease or 0% propensity to susceptibility to Alzheimer's disease. The reverse applies if the individual is homozygous for all susceptibility alleles. More typically, an individual is homozygous for resistance alleles at some loci, homozygous for susceptibility alleles at some loci, and heterozygous for resistance/susceptibility alleles at other loci. Such an individual's genetic propensity for Alzheimer's disease can be scored by assigning all resistance alleles a score of +1, and all susceptibility alleles a score of −1 (or vice versa) and combining the scores. For example, if an individual has 102 resistance alleles and 204 susceptibility alleles, the individual can be scored as having a 33% genetic propensity to resistance and 67% genetic propensity to susceptibility. Alternatively, homozygous resistance alleles can be assigned a score of +1, heterozygous alleles a score of zero and homozygous susceptibility alleles a score of −1. The relative numbers of resistance alleles and susceptibility alleles can also be expressed as a percentage. Thus, an individual who is homozygous for resistance alleles at 30 polymorphic sites, homozygous for susceptibility alleles at 60 polymorphic sites, and heterozygous at the remaining 63 sites is assigned a genetic propensity of 33% for resistance. As a further alternative, homozygosity for susceptibility can be scored as +2, heterozygosity, as +1 and homozygosity for resistance as 0.

The individual's score, and the nature of the polymorphic profile are useful in prognosis or diagnosis of an individual's susceptibility to Alzheimer's and related disease. Optionally, a patient can be informed of susceptibility to an Alzheimer's disease indicated by the genetic profile. Presence of a high genetic propensity to Alzheimer's disease can be treated as a warning to commence prophylactic or therapeutic treatment. Presence of a high propensity to disease also indicates the utility of performing secondary testing, such as testing brain activity by a psychometric test, such as the mini-mental exam, or taking a biopsy.

Polymorphic profiling is useful, for example, in selecting agents to effect treatment or prophylaxis of Alzheimer's in a given individual. Individuals having similar polymorphic profiles are likely to respond to agents in a similar way. Several drugs such as tacrine (Cognex), donepezil (Aricept), rivastigmine (Exelon), or galantamine (Reminyl), memantine (Namenda) (Forrest Laboratories), NSAIDs, statins, and Vitamin E have been reported to have some beneficial effect. Several other drugs are in clinical trials, such as, antigonadotropin-leuprolide (Voyager Pharmaceuticals), lecozotan SR (Wyeth), rasagiline mesylate (Eisai), TTP448 (TransTech Pharma), Ketasyn (Accera), Atomoxetine (Eli Lilly), AAB-001, an antibody to Aβ (Elan/Wyeth).

Polymorphic profiling is also useful for stratifying individuals in clinical trials of agents being tested for capacity to treat Alzheimer's or related conditions. Such trials are performed on treated or control populations having similar or identical polymorphic profiles (see EP99965095.5). Use of genetically matched populations eliminates or reduces variation in treatment outcome due to genetic factors, leading to a more accurate assessment of the efficacy of a potential drug. Computer-implemented algorithms can be used to identify more genetically homogenous subpopulations in which treatment or prophylaxis has a significant effect notwithstanding that the treatment or prophylaxis is ineffective in more heterogeneous larger populations. In such methods, data are provided for a first population with an Alzheimer's related disease treated with an agent, and a second population also with the disease but treated with a placebo. The polymorphic profile of individuals in the two populations is determined in at least one polymorphic site in or within 40 kb or preferably 10 kb of a gene selected from the group consisting of APOE, APOC, APP, C9orf52, CTNND2, CUGBP1, DKFZP566K1924, FARS1, FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC388110, MGC39715, NCE2, PFKFB2, PPP1R12B, PSEN1, TGD5, and TTLL2. Genes are preferably selected from the group consisting of APOE, APP, APOC1, CACNA1C, CKM, FARS1, KIAA1486, LOC147468, LOC166522, MGC39715, NCE2, PCBP3, PDE11A, PFKFB2, PVRL2, SEC13L1, TOMM40 and TTLL2 Data are also provided as to whether each patient in the populations reaches a desired endpoint indicative of successful treatment or prophylaxis. Subpopulations of each of the first and second populations are then selected such that the individuals in the subpopulations have greater similarity of polymorphic profiles with each other than do the individuals in the original first and second populations. There are many criteria by which similarity can be assessed. For example, one criterion is to require that individuals in the subpopulations have at least one susceptibility allele at each of at least ten of the above genes. Another criterion is that individuals in the subpopulations have at least 75% susceptibility alleles for each of the polymorphic sites at which the polymorphic profile is determined. Regardless of the criteria used to assess similarity, the endpoint data of the subpopulations are compared to determine whether treatment or prophylaxis has achieved a statistically significant result in the subpopulations. As a result of computer implementation, billions of criteria for similarity can be analyzed to identify one or a few subpopulations showing statistical significance.

Polymorphic profiling is also useful for excluding individuals not having Alzheimer's disease from clinical trials. Diagnosis of Alzheimer's disease is usually based on psychometric tests which are subject to identifying false positives. Including such individuals in the trial increases the size of the population needed to achieve a statistically significant result. Individuals not having Alzheimer's disease can be identified by determining the numbers of resistances and susceptibility alleles in a polymorphic profile as described above. For example, if a subject is genotyped at ten sites in ten genes of the invention associated with Alzheimer's disease, twenty alleles are determined in total. If over 50% and preferably over 60% or 75% percent of these are resistance genes, the individual is unlikely to have Alzheimer's disease and can be excluded from the trial regardless of the presence of symptoms that may appear to resemble Alzheimer's disease.

Polymorphic profiles can also be used after the completion of a clinical trial to elucidated differences in response to a given treatment. For example, the set of polymorphisms can be used to stratify the enrolled patients into disease sub-types or classes. It is also possible to use the polymorphisms to identify subsets of patients with similar polymorphic profiles who have unusual (high or low) response to treatment or who do not respond at all (non-responders). In this way, information about the underlying genetic factors influencing response to treatment can be used in many aspects of the development of treatment (these range from the identification of new targets, through the design of new trials to product labeling and patient targeting). Additionally, the polymorphisms can be used to identify the genetic factors involved in adverse response to treatment (adverse events). For example, patients who show adverse response may have more similar polymorphic profiles than would be expected by chance. This allows the early identification and exclusion of such individuals from treatment. It also provides information that can be used to understand the biological causes of adverse events and to modify the treatment to avoid such outcomes.

Polymorphic profiles can also be used for other purposes, including paternity testing and forensic analysis as described by U.S. Pat. No. 6,525,185. In forensic analysis, the polymorphic profile from a sample at the scene of a crime is compared with that of a suspect. A match between the two is evidence that the suspect in fact committed the crime, whereas lack of a match excludes the suspect. The present polymorphic sites can be used in such methods, as can other polymorphic sites in the human genome.

Polymorphic profiles can be used in further association studies of traits related to Alzheimer's disease including the Alzheimer's related diseases described above.

Although polymorphic profiling can be done at the level of individual polymorphic sites as described above, a more sophisticated analysis can be performed by analyzing haplotype blocks containing SNPs of the invention and/or others in linkage disequilibrium with them (see US 20040220750). In some instances, the boundaries of a haplotype block can be approximated by the length of a gene in which a polymorphic site occurs plus ten kb of flanking genomic sequence at either end. Each haplotype block can be characterized by two or more haplotypes (i.e., combinations of polymeric forms). In some instances, a haplotype can be determined by detecting a single haplotype-determining polymorphic form within a haplotype block. In other instances, multiple polymorphic forms are determined within the block (see Patil et al., Science Nov. 23, 2001;294(5547):1719-23). The haplotype at each of the haplotype blocks containing SNPs of the invention in an individual is a factor in determining resistance or susceptibility to an Alzheimer's related disease in an individual, and can be characterized as associating with resistance or susceptibility as can individual polymorphic forms. The number of haplotype blocks occupied by haplotypes associated with resistance and the number associated with susceptibility in a particular individual can be combined additively as for individual polymorphic forms to arrive at a percentage representing genetic propensity to resistance or susceptibility to an Alzheimer's related disease. The measure is more accurate than simply combining individual polymorphic forms because it gives the same weight to haplotype blocks containing multiple polymorphic sites as haplotype blocks within a single polymorphic site. The multiple polymorphic forms within the same block are associated with the same propensity for resistance or susceptibility to an Alzheimer's related disease, and should not be given the same weight as multiple polymorphic forms in different haplotype blocks, which indicate independent resistance or susceptibility to an Alzheimer's related disease.

The haplotype blocks used in polymorphic profiling preferably include one or more of the following genes: APOE, APP, APOC1, C9orf52, CTNND2, CUGBP1, DKFZP566K1924, FARS1, FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC388110, MGC39715, NCE2, PFKFB2, PPP1R12B, PSEN1, TGD5, and TTLL2. In some methods, each haplotype includes a different one of the above genes. In some methods, each haplotype group includes a different one of the following genes: APOE, APP, APOC1, CACNA1C, CKM, FARS1, KIAA1486, LOC147468, LOC166522, MGC39715, NCE2, PCBP3, PDE11A, PFKFB2, PVRL2, SEC13L1, TOMM40 and TTLL2, and in some methods APOC1, CACNA1C, CKM, FARS1, KIAA1486, LOC147468, LOC166522, MGC39715, NCE2, PCBP3, PDE11A, PFKFB2, PVRL2, SEC13L1, TOMM40 and TTLL2. Some methods determine a polymorphic profile in at least one polymorphic site in at least one haplotype block of the invention that does not include APOE. Some methods determine a polymorphic profile in at least one polymorphic site in at least one haplotype block of the invention that does not include APP, PSEN1 or APOE. Some methods determine a polymorphic profile in at least one polymorphic site in at least one haplotype block that does not include APP, PSEN1, APOE, TOMM40 or APOC1. Some method determine a polymorphic profile in at least one polymorphic site that does not occur within 40 kb of APP, PSEN1, APOE, TOMM40 or APOC1.

The methods of the invention detect haplotypes in at least 1, 2, 5, 10, 25, 50 or all of the haplotype blocks of the invention, preferably selected from the group consisting of A2BP1, C9orf52, CTNND2, CUGBP1, DKFZP566K1924, FARS1, FGL2, FLJ14442, FLJ36760, KIAA1486, KIAA1862, LNX2,LOC147468, LOC283867, LOC401237, LRP1B, MATN3, MRLC2, PCBP3, PDE11A, PVRL2, SEC13L1, SOX5 and TOMM40, and more preferably a group selected from APOE, APP, APOC1, CACNA1C, CKM, FARS1, KIAA1486, LOC147468, LOC166522, MGC39715, NCE2, PCBP3, PDE11A, PFKFB2, PVRL2, SEC13L1, TOMM40 and TTLL2. The haplotypes can be detected in combination with haplotypes at haplotype blocks other than those of the invention. However, the number of haplotype blocks is typically fewer than 1000 and often fewer than 100 or 50.

The invention also provides methods of expression profiling by determining levels of expression profiling of one or more genes of the invention i.e., APOE, APP, APOC1, C9orf52, CTNND2, CUGBP1, DKFZP566K1924, FARS1,FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC388110, MGC39715, NCE2, PFKFB2, PPP1R12B, PSEN1, TGD5, and TTLL2. The methods preferably determine expression levels of at least 2. 5. 10, 15, 20 or all of the above genes. Preferably, the methods determine expression levels of at least 2, 5, 10, 15 or 20 genes selected from the group consisting of: APOE, APP, APOC1, CACNA1C, CKM, FARS1, KIAA1486, LOC147468, LOC166522, MGC39715, NCE2, PCBP3, PDE11A, PFKFB2, PVRL2, SEC13L1, TOMM40 and TTLL2. Some methods determine the expression levels of at least one gene other than APP, PSEN1, and APOE. Some methods determine the expression levels of at least one gene other than APP, PSEN1, APOE1, TOMM40 and APOC1. Optionally, expression levels of other genes beyond those associated with Alzheimer's disease in the present application are also determined.

The expression levels of one or more genes in discrete sample (e.g., from a particular individual or cell line) are referred to as an expression profile. Typically, the expression profile is compared with an expression profile of the same genes in a control sample. The control sample can be a negative control (e.g., an individual (or population of individuals) not having or susceptible to an Alzheimer's related disease) or a positive control (e.g., an individual (or population of individuals) having or susceptible to an Alzheimer's related disease). The controls can be contemporaneous or historical. Individual expression levels in both the test and control samples can be normalized before comparison, e.g., by reference to the levels of a house keeping genes to avoid differences unrelated to the disease. The relative similarity of the expression profile of a test individual to the negative and positive control expression profiles is a measure of the individual's resistance or susceptibility to an Alzheimer's related disease. For example, if an expression profile is determined for ten genes of the invention, and the expression levels in the test subject are more similar to the positive control than the negative control for nine of the gene, one can conclude that the test individual has or is susceptible to an Alzheimer's related disease. The analysis can be performed at a more sophisticated level by weighting expression level according to where they lie between negative and positive controls. For example, if there is a large difference between negative and positive controls, and an expression level of a particular gene in a test individual lies close to the positive control that expression level is accorded greater weight than an expression level in a gene in which there is a smaller difference in expression levels between negative and positive controls, and the expression level of the test individual lies only slightly above the midpoint of the negative and positive control expression levels.

A variety of methods may be used to prognosticate and diagnose susceptibility or resistance to AD. The following methods are provided as examples and not as limitations of means to diagnose AD.

1. Detection of AD Nucleic Acids

Detection of presence or increased level of one or more nucleic acids, or fragments, derivatives, variants or complements thereof, associated with resistance to AD is a prognostic and diagnostic for resistance to AD. On the other hand, detection of presence or increased level of one or more nucleic acids, or fragments, derivatives, variants or complements thereof, associated with susceptibility to AD is a prognostic and diagnostic for susceptibility to AD. Similarly, detection of the presence of a genetic variant (e.g., SNP) associated with AD may be used to diagnose the disease, while detection of a variant correlated with resistance may be indicative of a healthy state.

Detection of nucleic acids and genetic variations in an individual may be made using any method known in the art. Examples of such methods include, for instance, Southern or Northern analyses, in situ hybridizations analyses, single stranded conformational polymorphism analyses, polymerase chain reaction analyses and nucleic acid microarray analyses. Such analyses may reveal both quantitative and qualitative aspects of the expression pattern of AD polypeptides. In particular, such analyses may reveal expression patterns or polypeptides associated with resistance or susceptibility to AD.

In one example, a diagnosis or prognosis is made using a test sample containing genomic DNA or RNA obtained from the individual to be tested. The individual can be an adult, child or fetus. The individual is preferably a human. The test sample can be from any source which contains genomic DNA or RNA including, e.g., blood, amniotic fluid, cerebrospinal fluid, skin, muscle, buccal or conjunctival mucosa, placenta, gastrointestinal tract or other organs. A test sample of DNA from fetal cells or tissue can be obtained by appropriate methods such as by amniocentesis or chorionic villus sampling. The test sample is subjected to one or more tests to identify the presence or absence of a nucleic acid of interest or a genetic variant of interest.

In one embodiment, Southern blot, northern blot or similar analyses methods are used to identify the presence or absence of a nucleic acid of interest or a genetic variant of interest using complementary nucleic acid probes associated with AD. The nucleic acid probes are preferably labeled before contacted with the sample.

In hybridization analysis, the sample is maintained under conditions sufficient to allow for specific hybridization of the nucleic acid probe to the target nucleic acid. In a preferred embodiment, the labeled nucleic acid probe and target nucleic acid specifically hybridize with no mismatches. Specific hybridization can be performed under stringent conditions disclosed herein and can be detected using standard methods. Hybridization is indicative of the presence or absence of a target nucleic acid. Specific hybridization to a nucleic acid or variant associated with resistance to AD is a diagnostic for resistance to AD. Specific hybridization to a nucleic acid or variant associated with susceptibility to AD is a diagnostic for susceptibility to AD. More than one probe can be used concurrently.

In a preferred embodiment, a nucleic acid probe is an allele-specific probe. See Saild, R. et al., (1986) Nature 324:163-166. Allele-specific probes can used to identify the presence or absence of one or more variants in a test sample of DNA obtained from an individual. A target nucleic acid is amplified using any method herein. Flanking sequences may also be amplified. In the case of Southern analysis, the amplified target nucleic acid is dot-blotted, using standard methods and the blot is then contacted with an allele specific nucleic acid probe. See Ausubel, F. et al., “Current Protocols in Molecular Biology” (eds. John Wiley & Sons). Detection of specific hybridization of an allele-specific probe to a target nucleic acid associated with resistance to AD is a diagnostic for resistance to AD. Detection of specific hybridization of an allele-specific probe to a target nucleic acid associated with susceptibility to AD is a diagnostic for susceptibility to AD.

Allele-specific probes are nucleic acids, mimetics, or a combination thereof, of approximately 10-50 base pairs or more preferably approximately 15-30 base pairs that specifically hybridize to one or more target nucleic acids. Target nucleic acids are any of the nucleic acids herein.

In one example, a target nucleic acid is a nucleic acid associated with resistance to AD. Nucleic acid probes that may be useful in identifying such target can be complementary to 1 or more, 2 or more, 3, or more, 4 or more, or 5 or more variants associated with resistance to AD. In another example, a target nucleic acid is a nucleic acid associated with susceptibility to AD. Nucleic acid probes that may be useful in identifying such target can be complementary to 1 or more, 2 or more, 3, or more, 4 or more, or 5 or more variants associated with susceptibility to AD. Such nucleic acid probes may be part of a set or in a kit (e.g., for use in Southern analysis or other techniques). Such nucleic acid probes can be allele-specific. Methods for preparing allele specific probes are known in the art.

One method for detecting nucleic acids associated with resistance or susceptibility to AD is northern analysis. Northern analysis can be used to identify gene expression patterns (e.g., levels of mRNA expression in different cell types or tissues, or during different developmental stages) of AD nucleic acids. See Ausubel, F. et al., “Current Protocols in Molecular Biology” (eds. John Wiley & Sons 1999). For northern analysis, a test sample of RNA is obtained from an individual by appropriate means. Specific hybridization of the test sample of RNA to a nucleic acid probe that is complementary to an RNA sequence associated with resistance to AD (e.g., encoding a polypeptide associated with resistance to AD) is a diagnostic or prognostic for resistance to AD. Specific hybridization of the test sample of RNA to a nucleic acid probe that is complementary to an RNA sequence associated with susceptibility to AD (e.g., encoding a polypeptide associated with susceptibility to AD) is a diagnostic or prognostic for susceptibility to AD. A nucleic acid probe is preferably labeled for northern blot analysis. A nucleic acid probe is preferably an allele-specific probe complementary to one or more of the variants (or polymorphisms) described in FIG. 1 or 2, or may include kits or collections of probes with more than one of such probes.

Alternative diagnostic and prognostic methods employ amplification of target nucleic acids associated with resistance or susceptibility to AD, e.g., by PCR. This is especially useful for target nucleic acids present in very low quantities. In one embodiment, amplification of target nucleic acids associated with resistance to AD indicates their presence and is a prognostic and diagnostic of resistance to AD. In a related embodiment, amplification of target nucleic acids associated with susceptibility to AD indicates their presence and is a prognostic and diagnostic of susceptibility to AD.

In another embodiment, cDNA is obtained from test sample RNA nucleic acids by reverse transcription. Nucleic acid sequences within the cDNA may be used as templates for amplification reactions. Nucleic acids used as primers in the reverse transcription and amplification reaction steps can be chosen from any of the nucleic acids herein. For detection of amplified products, the nucleic acid amplification may be performed using labeled primers or labeled nucleotides. Alternatively, enough amplified product may be made such that the product may be visualized by standard ethidium bromide staining or by utilizing other suitable nucleic acid staining methods. Alternatively, the amplified product may be labeled subsequent to the amplification reaction by conventional methods (e.g., end-labeling).

The above-described methods for determining expression patterns of AD genes may also be performed on an isolated cell population of a particular cell type derived from a given tissue. Additionally, in situ hybridization techniques may be utilized to provide information regarding which cells within a given tissue express an AD nucleic acid. Such analyses may provide information regarding a specific biological function of an AD nucleic acid, and any genes or genomic regions in linkage equilibrium therewith.

Microarrays can also be utilized for diagnosis and prognosis of resistance or susceptibility to AD. Microarrays comprise probes that are complementary to target nucleic acid sequences from an individual. A microarray probe is preferably allele-specific. In one embodiment, the microarray comprises a plurality of different probes, each coupled to a surface of a substrate in different known locations and each, capable of binding complementary strands. See, e.g., U.S. Pat. No. 5,143,854 and PCT Publication Nos. WO 90/15070 and WO 92/10092. These microarrays can generally be produced using mechanical synthesis methods or light directed synthesis methods that incorporate a combination of photolithographic methods and solid phase oligonucleotide synthesis methods. See Fodor et al., (1991) Science 251:767-777; and U.S. Pat. No. 5,424,186. Techniques for the mechanical synthesis of microarrays are described in, for example, U.S. Pat. No. 5,384,261.

Once a microarray is prepared, one or more target nucleic acids are hybridized to the microarray before the microarray is scanned. Typical hybridization and scanning procedures are described in PCT Publication Nos. WO 92/10092 and WO 95/11995, and U.S. Pat. No. 5,424,186. Briefly, target nucleic acid sequences that include one or more previously identified variants or polymorphisms are amplified and labeled by well-known techniques, such as attachment of a fluorescent moiety or using labeled primers during amplification (e.g. PCR). Primers that are complementary to both strands of the target sequence (one primer complementary to one strand upstream and the other primer complementary to the other strand downstream from a variant or polymorphism) may be used to amplify the target region. Asymmetric PCR techniques may be used. An amplified target, preferably incorporating a label, is then hybridized with the microarray under appropriate conditions. Upon completion of hybridization and washing of the microarray, the microarray is scanned to determine the position on the microarray to which the target sequence hybridizes. The hybridization data obtained from the scan is typically in the form of fluorescence intensities as a function of location on the microarray.

Although primarily described in terms of a single detection block, such as for the detection of a single polymorphism, microarrays can include multiple detection blocks, and thus be capable of analyzing multiple specific polymorphisms. In an alternative arrangement, detection blocks may be grouped within a single microarray or in multiple separate microarrays so that varying optimal conditions may be used during the hybridization of the target to the microarray. For example, it may be desirable to provide for the detection of polymorphisms that fall within G-C rich stretches of a genomic sequence separately from those that fall in A-T rich segments for optimization of hybridization conditions. Additional description of use of nucleic acid microarrays for detection of polymorphisms can be found, for example, in U.S. Pat. Nos. 5,858,659 and 5,837,832, the entire teachings of which are incorporated by reference herein.

Other methods to detect variant (or polymorphic) nucleic acids include, for example, direct manual sequencing (Church and Gilbert, (1988) Proc. Natl. Acad. Sci. USA 81:1991-1995; Sanger, F. et al. (1977) Proc. Natl. Acad. Sci. USA 74:5463-5467; and U.S. Pat. No. 5,288,644); automated fluorescent sequencing; single-stranded conformation polymorphism assays; clamped denaturing gel electrophoresis; denaturing gradient gel electrophoresis (Sheffield, V. C. et al. (1981) Proc. Natl. Acad. Sci. USA 86:232-236), mobility shift analysis (Orita, M. et al. (1989) Proc. Natl. Acad. Sci. USA 86:2766-2770), restriction enzyme analysis (Flavell et al. (1978) Cell 15:25; Geever, et al. (1981) Proc. Natl. Acad. Sci. USA 78:5081); heteroduplex analysis; Tm-shift genotyping (Germer et al. (1999) Genome Research 9:72-78); kinetic PCR (Germer et al. (2000) Genome Research 10:258-266); chemical mismatch cleavage (Cotton et al. (1985) Proc. Natl. Acad. Sci. USA 85:4397-4401); RNase protection assays (Myers, R. M. et al. (1985) Science 230:1242); and use of polypeptides which recognize nucleotide mismatches, such as E. coli mutS protein.

2. Detection of AD Polypeptides

Detecting the presence, level of expression, activity and location of AD polypeptides may be used as a diagnostic or prognostic for resistance or susceptibility to AD. Briefly, detection of the presence, level of expression or enhanced activity of polypeptides associated with resistance to AD is a diagnostic and prognostic for resistance to AD. Detection of the presence, level of expression or enhanced activity of polypeptides associated with susceptibility to AD is a diagnostic and prognostic for susceptibility to AD.

Proteins may be analyzed from any tissue or cell type, and in some specific embodiments neuronal tissues are used. Analyses can be made in vivo or in vitro. In a preferred embodiment a biopsy (or tissue sample) is obtained from brain tissue (e.g., from the hippocampus, or the neocortex, or the parietal or temporal lobes) of an individual to be tested.

Methods to detect and isolate polypeptides include, for example, enzymes linked immunosorbent assays (ELISAs), immunoprecipitations, immunofluorescence, immunoblotting, Western blotting, spectroscopy, colorimetry, electrophoresis and isoelectric focusing. See U.S. Pat. No. 4,376,110; see also Ausubel, F. et al., “Current Protocols in Molecular Biology” (Eds. John Wiley & Sons, chapter 10). Protein detection and isolation methods employed may also be those described in Harlow and Lane (Harlow, E. and Lane, D., “Antibodies: A Laboratory Manual,” Cold Spring Harbor Laboratory Press, Cold Spring Harbor, N.Y., 1998).

In one embodiment, the presence, amount and location of polypeptides associated with resistance to AD can be determined using a probe or an antibody that specifically binds one or more polypeptides associated with resistance to AD. In another embodiment, the presence, absence, amount or location of a polypeptide associated with susceptibility to AD can be determined using a probe or antibody that specifically bind one or more polypeptides associated with susceptibility to AD.

Antibodies, such as those described herein may be used to determine the presence of a polypeptide associated with resistance or susceptibility to AD.

In a preferred embodiment, a probe or antibody is labeled directly or indirectly. Direct labeling involves coupling (physically linking) a detectable substance to an antibody or a probe. Indirect labeling involves the reactivity of the probe with another reagent that is directly labeled. Examples of indirect labeling include, for example, detection of a primary antibody using a fluorescently labeled secondary antibody and end labeling of a DNA probe with biotin such that it can be detected with fluorescently labeled streptavidin.

A solid support may be utilized to immobilize either the antibody or probe or the sample (e.g., AD polypeptide). In one example, a sample may be immobilized onto a solid support such as nitrocellulose, which is capable of immobilizing cells, cell particles, or soluble proteins. The support may then be washed with suitable buffers followed by treatment with a detectably labeled antibody. The amount of bound labeled antibody on the solid support may then be detected by conventional means. Well known supports include glass, polystyrene, polypropylene, polyethylene, dextran, nylon, amylases, natural and modified celluloses, polyacrylamides, gabbros, and magnetite.

The antibodies herein can be linked to an enzyme and used in an enzyme immunoassay. See Voller, “The Enzyme Linked Immunosorbent Assay (ELISA)”, Diagnostic Horizons 2:1-7 (Microbiological Associates Quarterly Publication, Walkersville, Md. 1978); Maggio, “Enzyme Immunoassay” (CRC Press, Boca Raton, Fla. 1980); Ishikawa, et al., “Enzyme Immunoassay” (Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes that can be used to label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Detection can be accomplished by calorimetric methods which employ a chromogenic substrate for the enzyme. Detection can also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect wild type or mutant peptides through the use of a radioimmunoassay. See Weintraub, B., “Principles of Radioimmunoassays, Seventh Training Course on Radioligand Assay Techniques” (The Endocrine Society, March, 1986). The radioactive isotope can be detected by such means as the use of a gamma counter or a scintillation counter or by autoradiography.

It is also possible to label the antibody with a fluorescent compound. When the fluorescently labeled antibody is exposed to light of the proper wavelength, its presence can be detected my measuring emitted fluorescence. Among the most commonly used fluorescent labeling compounds are fluorescein isothiocyanate, rhodamine, phycoerythrin, phycocyanin, allophycocyanin, o-phthaldehyde and fluorescamine. The fluorescently labeled antibody can be coupled with light microscopic, flow cytometric or fluorimetric detection. In one example, antibodies, or fragments thereof, may be employed histological, as in immunofluorescence or immunoelectron microscopy, for in situ detection of a polypeptide associated with resistance or susceptibility to AD. In situ detection may be accomplished by removing a histological specimen from a patient, such as by biopsy. The specimen is then contacted with a labeled antibody described herein. The antibody or fragment is preferably contacted by overlaying the labeled antibody or fragment onto the sample. This procedure allows for the determination of the presence, absence, amount and location of a polypeptide of interest.

The antibody can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibodies herein. Bioluminescence is a type of chemiluminescence found in biological systems in which a catalytic protein increases the efficiency of the chemiluminescent reaction. The presence of a bioluminescent protein is determined by detecting the presence of luminescence. Preferred bioluminescent compounds for purposes of labeling antibodies are luciferin, luciferase and aequorin.

In one embodiment, the presence (or absence) of a polypeptide associated with AD in a sample (e.g., a cell, cell lysate, tissue, whether in vivo or in vitro) can be established by contacting the sample with an antibody and then detecting a binding complex. The presence of a polypeptide associated with resistance to AD is a diagnostic and prognostic of resistance to AD or more particularly LOAD. The presence of a polypeptide associated with susceptibility to AD is a prognosis and diagnosis of susceptibility to AD.

In another embodiment, the level of expression or sequence of a polypeptide associated with AD in a test sample is compared with the level of expression or sequence of the same polypeptide in a control sample. A control sample may have a known level of expression of the polypeptide, and/or can be a sample from a healthy individual or from a different tissue or organ from the test individual.

Alterations in the level of expression or sequence of an AD polypeptide may be indicative of susceptibility or resistance to AD. In one example, a test sample from an individual is assessed for a change in expression (e.g., level of transcription or translation) and/or sequence (e.g., splicing variants, polymorphisms) of a polypeptide associated with susceptibility to AD. Detection of an increased level of expression of a polypeptide associated with susceptibility to AD may be a prognosis or diagnosis of, for example, an onset of AD or an increased susceptibility to related disease. On the contrary, detection of a reduced level of a polypeptide associated with susceptibility to related disease may be indicative of, for example, a reduced susceptibility to AD or an effective treatment against AD (e.g., if the test sample is from an individual after treatment and the control sample is from the same individual before treatment). Detection of an increased level of a polypeptide associated with resistance to AD may be a prognosis or diagnosis of, for example, increased immunity to AD or an effective treatment regimen against AD. On the other hand, detection of a reduced level of a polypeptide associated with resistance to AD may be a prognosis or diagnosis of, for example, decreased immunity to AD or an ineffective treatment regimen against AD. Similarly, detection of an increase in compositions (including, e.g., peptides, derivatives, variants, splicing variants) associated with susceptibility to AD is a prognosis or diagnosis of an earlier onset or more severe symptoms of AD while detection of an increase in compositions associated with resistance to AD is a prognosis or diagnosis for immunity or reduced risk for developing AD.

Further, it may be useful to compare the level of expression of a reference AD polypeptide to the level of expression of an alternate or variant AD polypeptide in a cell or tissue that is heterozygous for a nonsynonymous polymorphism in a coding region. Such a cell or tissue may be expected to produce equivalent amounts of both the reference and alternate polypeptides encoded by the coding region. However, if measurement of the amounts of these two polypeptides indicates that one is produced at a statistically higher level than the other, then this is an indication that there is another regulatory mechanism at play. For example, it may be in indication that the coding region is exhibiting differential allelic expression, expressing one allele at a higher level than the other; that the RNA from one allele is being processed differently than the RNA for the other allele (e.g., via degradation, splicing, translation, etc.); or that the reference polypeptide is being processed differently than the alternate polypeptide (e.g., via degradation, post-translational modification, etc.)

Kits useful in diagnosis and prognosis include reagents comprising, for example, instructions for use and analysis; means for collecting a tissue or cell sample; nucleic acid probes or primers (e.g., for amplification, reverse transcriptase and detection); labels (e.g., for nucleic acids or proteins); microarrays, gels, membranes or other detection apparati; restriction enzymes (e.g., for RFLP analysis); allele-specific probes; antisense nucleic acids; antibodies; and other protein binding probes, any of which may be labeled.

V Screening Assays and Agents

The invention provides methods to identify agents potentially useful in diagnosis, prognosis, prophylaxis or treatment of an Alzheimer's related disease. Agents are tested for their capacity to modulate expression or activity of a gene selected from the group consisting of APOE, APP, APOC1, C9orf52, CTNND2, CUGBP1, DKFZP566K1924, FARS1, FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC388110, MGC39715, NCE2, PFKFB2, PPP1R12B, PSEN1, TGD5, and TTLL2, preferably a gene shown in Table 9. In some methods, the gene is other than APOE, APP and PSEN1. In some methods, the gene is selected from the group: APOC1, CACNA1C, CKM, FARS1, KIAA1486, LOC147468, LOC166522, MGC39715, NCE2, PCBP3, PDE11A, PFKFB2, PVRL2, SEC13L1, TOMM40 and TTLL2. Expression assays are usually performed in cell culture, but can also be performed in animal models or in an in vitro transcription/translation system. The cell culture can be of primary cells, particularly, those known or suspected to have a role in Alzheimer's disease, such as neurons or cells transfected with a gene of the above group. In the latter case, the coding portion of the gene is typically transfected with its naturally associated regulatory sequences, so as to permit expression of the gene in the transfected cell. However, the coding portion of the gene can also be operably linked to regulatory sequences from other (i.e., heterologous) genes. Optionally, the protein encoded by the gene is expressed fused to a tag or marker to facilitate its detection. The compound to be screened is introduced into the cell, usually in the form of a DNA molecule that can be expressed or directly as an RNA or protein. Expression of the gene can be detected either at the mRNA or protein level. Expression at the mRNA level can be detected by a hybridization assay, and at the protein level by an immunoassay. Detection of the protein level is facilitated by the presence of a tag. Similar screens can be performed in an animal, either natural or transgenic, or in vitro. Expression levels in the presence of an agent under test are compared with those in a control assay in the absence of compound, an increase or decrease in expression indicating that the compound modulates activity of the gene.

Assays to detect modulation of a protein encoded by a gene of the invention can also be performed. In some instances, a preliminary assay is performed to detect specific binding between an agent and a protein encoded by a gene of the invention. A binding assay can be performed between the agent and a purified protein, of if the protein is expressed extracellularly, between the agent and the protein expressed from a cell. Optionally, either the agent or protein can be immobilized before or during the assay. Such an assay reduces the pool of candidate agents for an activity assay. The nature of the activity assay depends on the activity of the gene.

Agents that modulate expression or activity of the genes of the invention can then be tested in animal models for Alzheimer's or related diseases. The animal models can be transgenic (as described below) or nontransgenic. Agents are tested in comparison with otherwise similar control assays except for the absence of the compound being tested. A reduction or inhibition of a sign or symptom of disease by an agent relative to a control indicates an agent has pharmacological activity potentially useful in treating the disease. The animal models used for such testing can be the novel animal models described below or conventional animal models of Alzheimer's disease of which many are known. Such models include, for example, mice bearing a 717 (APP770 numbering) mutation of APP described by Games et al., supra, and mice bearing a 670/671 (APP770 numbering)Swedish mutation of APP such as described by McConlogue et al., U.S. Pat. No. 5,612,486 and Hsiao et al., Science, 274, 99 (1996).

Agents that modulate expression or activity of the genes of the invention can also be screened in similar fashion in animal models of other diseases, particularly other Alzheimer's related diseases. For example, use of an animal model of Parkinson's disease is described by Hashimoto et al., Ann. N.Y. Acad. Sci. 991:171-88 (2003), and of prion disease by Barrett et al., J. Virol. 77(15):8462-9 (2003), and of ALS by Kilveny et al. Nature Medicine 5, 347-350 (1999).

Examples of agents include, but are not limited to: transcription factors, binding molecules, antisense nucleic acids, PNAs, mimetics, small or large organic or inorganic molecules, polypeptides (e.g., soluble peptides, or Ig-tailed fusion peptides), antibodies, as described above, (e.g., monoclonal, polyclonal, humanized, anti-idiotypic, chimeric or single chain antibodies, Fab, F(ab′)2, Fab expression library fragments, and epitope-binding fragments thereof), fusion proteins, prodrugs, drugs in trials, previously approved drugs for AD, drugs developed for indications other than AD, and any fragments, derivatives, variants or complements of any of the above. Such agents can be used separately or in combination.

Agents can be obtained from natural sources, such as, e.g., marine microorganisms, lgae, plants, and fungi. Alternatively, agents can be from combinatorial libraries of agents, including peptides or small molecules, or from existing repertories of chemical compounds synthesized in industry, e.g., by the chemical, pharmaceutical, environmental, agricultural, marine, cosmeceutical, drug, and biotechnological industries. Agents can include, e.g., pharmaceuticals, therapeutics, environmental, agricultural, or industrial agents, pollutants, cosmeceuticals, drugs, organic compounds, lipids, glucocorticoids, antibiotics, peptides, proteins, sugars, carbohydrates, and chimeric molecules.

Combinatorial libraries can be produced for many types of agents that can be synthesized in a step-by-step fashion. Such agents include polypeptides, proteins, nucleic acids, beta-turn mimetics, polysaccharides, phospholipids, hormones, prostaglandins, steroids, aromatic compounds, heterocyclic compounds, benzodiazepines, oligomeric N-substituted glycines and oligocarbamates. Large combinatorial libraries of compounds can be constructed by the encoded synthetic libraries (ESL) method described in Affymax, WO 95/12608, Affymax WO 93/06121, Columbia University, WO 94/08051, Pharmacopeia, WO 95/35503 and Scripps, WO 95/30642 (each of which is incorporated herein by reference in its entirety for all purposes). Peptide libraries can also be generated by phage display methods. See, e.g., Devlin, WO 91/18980. Compounds to be screened can also be obtained from governmental or private sources, including, e.g., the National Cancer Institute's (NCI) Natural Product Repository, Bethesda, Md., the NCI Open Synthetic Compound Collection, Bethesda, Md., NCI's Developmental Therapeutics Program, or the like.

The compounds also include several categories of molecules known to regulate gene expression, such as zinc finger proteins, ribozymes, siRNAs and antisense RNAs. Zinc finger proteins can be engineered or selected to bind to any desired target site within a gene of the invention. An exemplary motif characterizing one class of these proteins (C₂H₂ class) is -Cys-(X)₂₄-Cys-(X)₁₂-His-(X)₃₋₅-His (where X is any amino acid). A single finger domain is about 30 amino acids in length, and several structural studies have demonstrated that it contains an alpha helix containing the two invariant histidine residues and two invariant cysteine residues in a beta turn co-ordinated through zinc. In some methods, the target site is within a promoter or enhancer. In other methods, the target site is within the structural gene. In some methods, the zinc finger protein is linked to a transcriptional repressor, such as the KRAB repression domain from the human KOX-1 protein (Thiesen et al., New Biologist 2, 363-374 (1990); Margolin et al., Proc. Natl. Acad. Sci. USA 91, 4509-4513 (1994); Pengue et al., Nucl. Acids Res. 22:2908-2914 (1994); Witzgall et al., Proc. Natl. Acad. Sci. USA 91, 4514-4518 (1994)). In some methods, the zinc finger protein is linked to a transcriptional activator, such as VIP16. Methods for selecting target sites suitable for targeting by zinc finger proteins, and methods for design zinc finger proteins to bind to selected target sites are described in WO 00/00388. Methods for selecting zinc finger proteins to bind to a target using phage display are described by EP.95908614.1. The target site used for design of a zinc finger protein is typically of the order of 9-19 nucleotides.

Agents identified via these assays can be utilized to prevent, treat, diagnose and prognosticate AD. For example, when AD results from an overall lower level or activity of RNAs or polypeptides associated with resistance to AD, agents that enhance or stimulate the expression or activity of such RNAs or polypeptides may be used to treat or prevent AD. When AD results from the an overall higher level or activity of RNAs or polypeptides associated with susceptibility to AD, agents that inhibit or diminish the expression or activity of such RNAs or polypeptides may be used to treat or prevent AD. Optionally, an agent that modulates expression of a gene can be combined with an agent that modulates activity of a protein encoded by the gene. Optionally, agents that modulate expression of different genes which in combination result in Alzheimer's disease can be combined.

1. Screening Assays for Agents that Modulate the Expression of Coding Nucleic Acids

In one embodiment, agents that modulate (enhance, inhibit, or otherwise change) the level of expression of an AD polypeptide can be identified by comparing the level of expression of such coding nucleic acid in the presence of a test agent and in a control. A modulation of expression may occur at the DNA level (e.g., a transcription factor, etc.) or at the RNA level (antisense RNA, splicing, RNA-binding protein, etc.) A control can be in the absence of the test agent or a previously established level of expression. A solution or sample (e.g., cell or tissue culture) containing nucleic acids encoding an AD polypeptide can be contacted with a test agent. A solution can comprise, for example, cells or cell lysates containing the AD gene as well as other elements necessary for transcription/translation. Cells not suspended in solution as well as animal models may also be used. In addition, complexes of nucleic acid and protein agents may be detected by methods well known in the art. For example, such methods may utilize chromatography, microarrays, fluorescent labeling, and other methods further described in the section entitled Immobilization Assays herein.

If the level of expression of the AD coding nucleic acid is greater by an amount that is statistically significant from the level of expression in the control, then the test agent is an agonist of AD gene expression or activity. If the level of expression in the presence of the test agent is less by an amount that is statistically significant from the level of expression in the control, then the test agent is an antagonist of the expression of associated gene. The level of expression coding nucleic acids can be evaluated, for example, by determining the level of mRNA or polypeptides that are expressed, and/or any other method herein or known in the art, including but not limited to Northern analysis, Western blotting and antibodies.

Using a similar method, agents that modulate the expression of associated gene variants associated with resistance or with susceptibility to AD can be identified. Preferably an agent is an agonist to the expression of associated genomic region variants associated with resistance to AD or an antagonist to the expression of associated genomic region variants associated with susceptibility to AD. More preferably, an agent is both an agonist to the expression of associated genomic region variants associated with resistance to AD and an antagonist of associated genomic region variants associated with susceptibility to AD.

2. Screening Assays for Agents that Modulate the Expression of Coding Nucleic Acids by Interacting with Regulatory Regions

In another embodiment, agents that modulate the expression of coding AD nucleic acids by interacting with an AD regulatory region (e.g., enhancers, introns, 5′ and 3′ untranslated regions (e.g., promoters) and uORF's) are provided. For example, agents that modulate transcription or translation of nucleic acids herein (e.g., transcription factors) can be identified by contacting a solution containing non-coding nucleic acids associated with AD operably linked to a reporter gene with a test agent. After contact with the test agent, the level of expression of the reporter gene (e.g., the level of mRNA or polypeptide expressed) is assessed and compared with the level of expression in a control (e.g., the level of expression in the absence of a test agent or a level of expression that has previously been established). If the level of expression in the test sample is greater than the level of expression in the control sample by a statistically significant amount, then the test agent is an agonist of expression. If the level of expression in the test sample is less than the level of expression in a control sample by a statistically significant amount, then the test agent is an antagonist of the expression.

In some embodiments, an agent is an antagonist to the expression of associated genomic region variants associated with susceptibility to AD. In other embodiments, an agent is an agonist to the expression of associated genomic region variants associated with resistance to AD. In further embodiments, an agent is both an antagonist to the expression of AD variants associated with susceptibility to AD and an agonist to the expression of AD variants associated with resistance to AD. In particular embodiments, the agent increases the resistance and/or decreases the susceptibility of an organism (e.g., human) to AD by interacting with one or more AD regulatory nucleic acids.

3. Screening Assays for Agents that Enhance/Inhibit Polypeptide Activity

In another embodiment, agents that modulate (enhance, inhibit, or otherwise alter) the activity of polypeptides associated with AD (e.g., enhance the presence of certain splicing variants, or modulate one or more functions of the polypeptide (e.g., binding activity)) are identified by contacting a test agent with a cell, cell lysate or a solution containing nucleic acids and/or polypeptides associated with AD and comparing the activity of the polypeptides with their activity in a control (in absence of the test agent or a previously established level activity). If the activity of polypeptides associated with AD is enhanced by an amount that is statistically significant from the level of activity of the same polypeptides in a control, then the agent is an agonist of the activity of such polypeptides. If the activity of polypeptides associated with AD is inhibited by an amount that is statistically significant from the level of activity of the same polypeptides in a control, then the agent is an antagonist of the activity of such polypeptides. The activity of AD polypeptides may be modulated, e.g., by enhancing or inhibiting the expression of such polypeptides (i.e., increasing or decreasing the production of the polypeptides); by enhancing or inhibiting the activity of one or more such polypeptides (e.g., by altering the enzyme kinetics, binding affinity, etc. of the polypeptides); or by changing the cellular localization of one or more of such polypeptides.

In a preferred embodiment, an agent is an agonist of the activity of polypeptides associated with resistance to AD. In another preferred embodiment, an agent is an antagonist of the activity of polypeptides associated with susceptibility to AD. Preferably, an agent is both an agonist of the activity of polypeptides associated with resistance to AD and an antagonist of the activity of polypeptides associated with susceptibility to AD.

4. Protein Agents that Bind AD Polypeptides

In another embodiment, assays can be used to identify protein agents that interact or bind one or more of the polypeptides herein, e.g., an AD polypeptide. Any method suitable for detecting protein-protein interactions may be employed for identifying protein agents that interact with or bind to AD polypeptides. Among the traditional methods that may be employed are co-immunoprecipitation, crosslinking, and co-purification through gradients or chromatographic columns.

In one embodiment, a yeast two-hybrid system, such as that described by Fields and Song (Fields, S. and Song, O., (1989) Nature 340:245-246), can be used to identify polypeptides that interact with one or more AD variants. A yeast two-hybrid system employs two vectors. The first vector has a DNA binding domain; the second, a transcription activation domain. Each domain is fused to a sequence encoding a different polypeptide. If the polypeptides interact with one another, transcriptional activation can be achieved, and transcription of specific markers can be used to identify the presence of interaction and transcriptional activation. In one example, a first vector contains a nucleic acid encoding a DNA binding domain and an AD polypeptide, and a second vector contains a nucleic acid encoding a transcription activation domain and test polypeptide which may potentially interact with the AD polypeptide (e.g., a binding agent). Incubation of yeast containing the first vector and the second vector under appropriate conditions (e.g., mating conditions such as those used in the Matchmaker system from Clontech (Palo Alto, Calif.)) allows for the identification of colonies that express the markers of interest. These colonies can be examined to identify the polypeptide(s) that interact with the AD polypeptide tested. The binding molecules may be use as agents to alter the activity or expression of an AD polypeptide as described above.

In another embodiment, a protein microchip may be used to identify polypeptides that bind to AD polypeptides or any other polypeptide herein. A protein microchip or microarray is provided having one or more protein complexes and/or antibodies selectively immunoreactive with a polypeptide of interest. Protein microarrays are becoming increasingly important in both proteomics research and protein-based detection and diagnosis of diseases. The protein microarrays in accordance with this embodiment are be useful in a variety of applications including, e.g., large-scale or high-throughput screening for compounds capable of binding to the protein complexes or modulating the interactions between the interacting protein members in the protein complexes.

Protein microarrays can be prepared in a number of methods known in the art. An example of a suitable method is that disclosed in MacBeath and Schreiber, (2002) Science, 289:1760-1763. Essentially, glass microscope slides are treated with an aldehyde-containing silane reagent (SuperAldehyde Substrates purchased from TeleChem International, Cupertino, Calif.). Nanoliter volumes of protein samples in a phosphate-buffered saline with 40% glycerol are then spotted onto the treated slides using a high-precision contact-printing robot. After incubation, the slides are immersed in a bovine serum albumin (BSA)-containing buffer to quench the unreacted aldehydes and to form a BSA layer that functions to prevent non-specific protein binding in subsequent applications of the microchip. Alternatively, as disclosed in MacBeath and Schreiber, proteins or protein complexes of the present invention can be attached to a BSA-NHS slide by covalent linkages. BSA-NHS slides are fabricated by first attaching a molecular layer of BSA to the surface of glass slides and then activating the BSA with N,N′-disuccinimidyl carbonate. As a result, the amino groups of the lysine, aspartate, and glutamate residues on the BSA are activated and can form covalent urea or amide linkages with protein samples spotted on the slides. See MacBeath and Schreiber, Science, 289:1760-1763 (2000).

Another example of a useful method for preparing a protein microchip is disclosed in PCT Publication Nos. WO 00/4389A2 and WO 00/04382. First, a substrate or chip base is covered with one or more layers of thin organic film to eliminate any surface defects, insulate proteins from the base materials, and to ensure uniform protein array. Next, a plurality of protein-capturing agents (e.g., antibodies, peptides, etc.) are arrayed and attached to the base that is covered with the thin film. Proteins or protein complexes can then be bound to the capturing agents forming a protein microarray. The protein microchips are kept in flow chambers with an aqueous solution.

The protein microarrays herein can also be made by the method disclosed in PCT Publication No. WO 99/36576, which is incorporated herein by reference. For example, a three-dimensional hydrophilic polymer matrix, i.e., a gel, is first dispensed on a solid substrate such as a glass slide. The polymer matrix gel is capable of expanding or contracting and contains a coupling reagent that reacts with amine groups. Thus, proteins and protein complexes can be contacted with the matrix gel in an expanded aqueous and porous state to allow reactions between the amine groups on the protein or protein complexes with the coupling reagents thus immobilizing the proteins and protein complexes on the substrate. Thereafter, the gel is contracted to embed the attached proteins and protein complexes in the matrix gel.

The protein microchips of the present invention can also be prepared with other methods known in the art, e.g., those disclosed in U.S. Pat. Nos. 6,087,102, 6,139,831, 6,087,103; PCT Publication Nos. WO 99/60156, WO 99/39210, WO 00/54046, WO 00/53625, WO 99/51773, WO 99/35289, WO 97/42507, WO 01/01142, WO 00/63694, WO 00/61806, WO 99/61148, WO 99/40434, all of which are incorporated herein by reference.

5. Agents that Interfere with AD Interaction with Binding Agents

The polypeptides herein may interact in vivo with one or more cellular or extracellular binding agents (e.g., polypeptides, nucleic acids, etc.) to form a complex. Agents that disrupt such an interaction may be used to regulate the activity or function of the AD polypeptides herein. Such agent may include, but are not limited to molecules such as antibodies, peptides, and the like. Assays that assess the impact of a test agent on the activity of an AD polypeptide in relation to a cellular or extracellular binding agent are provided. These assays involve the preparation of a reaction mixture containing an AD polypeptide and a cellular or extracellular binding agent and a time sufficient to allow the two products to interact and bind thus forming a complex.

To test an agent for inhibitory activity, reaction mixtures are prepared in the presence and absence of the test agent. The test agent can be initially included in the reaction mixture or added at a time subsequent to the addition of the AD polypeptide and/or its cellular or extracellular binding agent. Control reaction mixtures can be incubated without the test agent or with a placebo agent. Formation of complexes between AD polypeptides and cellular or extracellular binding agents is measured in both in the control and test reaction mixtures. A difference in the formation of a complex in the control reaction and the test reaction mixture indicates that the compound affects the interaction of the AD polypeptide and the cellular or extracellular binding agent. For example, the agent may enhance or inhibit binding between the PD-related disease polypeptide and the binding agent. Additionally, complex formation in a reaction mixture containing a test agent and AD polypeptide may be compared to complex formation in a reaction mixture containing the test agent and a second AD polypeptide that is encoded by a different nucleic acid sequence than the first AD polypeptide. In certain embodiments, the first and second AD polypeptides are encoded by different alleles of the same gene. This comparison can be important in those cases in which it is desirable to identify agents that disrupt interaction of a particular AD polypeptide.

The screening assays for test agents that interfere with AD polypeptide interaction with binding agents may be conducted in a heterogeneous or homogeneous format. Heterogeneous assays involve anchoring one of the binding partners onto a solid phase and detecting complexes anchored on the solid phase at the end of the reaction. In homogeneous assays, the entire reaction is carried out in a liquid phase. In either approach, the order of addition of reactants can be varied to obtain different information about the agents being tested. In either example, test agents that affect the interaction between the AD polypeptides and the cellular or extracellular binding agents can be tested, for example, by competition by adding the test agent to the reaction mixture prior to, post, or simultaneously within the AD polypeptide and cellular or extracellular binding agents and assessing the difference in complex formation. Alternatively, test agents that disrupt or otherwise affect formed complexes, (e.g., compounds (e.g., with higher binding constants) that displace one of the components from the complex) can be tested by adding the test agent to the reaction mixture after the complexes have been formed.

The ability or effectiveness of a test agent to bind to an AD polypeptide, a cellular or extracellular binding agent, or a complex thereof can be assessed, for example, by coupling a test agent with a radioisotope or enzymatic label such that binding of the test agent to the AD polypeptide, binding agent, or complex thereof can be determined by detecting the label (e.g., 125I, 35S, 14C, or 3H) either directly or indirectly (e.g., by direct counting of radio emission or by scintillation counting). Alternatively, test agents can be enzymatically labeled with, for example, horseradish peroxidase, alkaline phosphatase or luciferase and the enzymatic label can be detected by determination of conversion of an appropriate substrate to a product.

In another embodiment, the ability of a test agent to interact with an AD polypeptide, binding agent, or complex thereof can be assessed without the labeling of any of the interactants. For example, a microphysiometer can be used to detect the interaction of a test agent with an AD polypeptide or a binding agent without the labeling of either the test agent, the AD polypeptide or the binding agent. See McConnell, H. M. et al. (1992) Science 257:1906-1912. As used herein, a “microphysiometer” (e.g., Cytosensor (Molecular Devices, Sunnyvale, Calif.)) is an analytical instrument that measures the rate at which a cell acidifies its environment using a light-addressable potentiometric sensor (LAPS). Changes in this acidification rate can be used as an indicator of the interaction between the binding agent and the AD polypeptide.

6. Screening for Small Molecules

Agents that enhance or inhibit the expression, function, and/or activity of AD nucleic acids or polypeptides can be obtained using any of the numerous approaches in combinatorial library methods known in the art, including: biological libraries; natural products libraries; spatially addressable parallel solid phase or solution phase libraries; synthetic library methods requiring deconvolution; the ‘one-bead one-compound’ library method; and synthetic library methods using affinity chromatography selection. The biological library approach is largely limited to polypeptide libraries, while the other four approaches are applicable to polypeptide, non-peptide oligomer or small molecule libraries of compounds. See Lam, K. S. (1997) Anticancer Drug Des. 12:145.

Non-peptide agents or small molecules are generally preferred because they are more readily absorbed after oral administration and have fewer potential antigenic determinants. Small molecules are also more likely to cross the blood brain barrier than larger protein-based pharmaceuticals. Methods for screening small molecule libraries for candidate protein-binding molecules are well known in the art and may be employed to identify molecules that modulate (e.g., through direct or indirect interaction) one or more of the AD polypeptides herein. Briefly, AD polypeptides may be immobilized on a substrate and a solution including the small molecules is contacted with the AD polypeptide under conditions that are permissive for binding. The substrate is then washed with a solution that substantially reflects physiological conditions to remove unbound or weakly bound small molecules. A second wash may then elute those compounds that are bound strongly to the immobilized polypeptide. Alternatively, the small molecules can be immobilized and a solution of AD polypeptides can be contacted with the column, filter or other substrate on which the small molecules are immobilized. The ability to detect binding of an AD polypeptide to a small molecule may be facilitated by labeling (e.g., radio-labeling or chemiluminescence) the polypeptide or small molecule.

In another embodiment, electronic molecular modeling applies an algorithm to screen small molecule databases for ligands and molecules that interact or bind with AD polypeptides or those in pathways therewith. See Meng et al., (1992) J. Comp. Chem. 15:505. In one example the DOCK3.5 is used to screen for small molecules that interact with AD polypeptides, preferably the binding pocket of an AD polypeptide. A “negative image” of the binding pocket on a protein surface is created. The image is created by the computational equivalent of placing atom-sized spheres into the binding pocket. A representative set of spheres are identified by DOCK3.5 that fit extremely well into the binding pocket. The generated spheres constitute an irregular grid that is matched to the atomic centers of potential ligands. The list of atom centers, or more conveniently the matrix of interatomic distances linking these atom centers forms a useful description of the binding site. The matrix of interatomic distances for the putative ligand is also made. The best mutual overlap of the two matrices is sought. This alignment specifies the orientation of the ligand relative to the negative image of the protein and thus docks the ligand into the protein's binding pocket.

Non-peptide agents or small molecule libraries can be prepared by a synthetic approach, but recent advances in biosynthetic methods using enzymes may enable one to prepare chemical libraries that are otherwise difficult to synthesize chemically. Small molecule libraries can also be obtained from various commercial entities, for example, SPECS and BioSPEC B.V. (Rijswijk, the Netherlands), Chembridge Corporation (San Diego, Calif.), Comgenex USA Inc., (Princeton, N.J.), Maybridge Chemical Ltd. (Cornwall, U.K.), and Asinex (Moscow, Russia). These small molecule libraries can be screening in a high throughput manner to identify one or more agents. For example, a high throughput screening assay for small molecules that was disclosed in Stockwell, B. R. et al., Chem. & Bio., (1999) 6:71-83, is a miniaturized cell-based assay for monitoring biosynthetic processes such as DNA synthesis and post-translational processes.

7. Immobilization Assays

In any embodiment herein, it may be desirable to immobilize either the AD polypeptides, the test agent or other components of the assay (e.g., binding agents) on a substrate to facilitate the separation of bound polypeptides from unbound polypeptides, as well as to accommodate automation of the assay. A substrate can be any vessel suitable for containing the reactants. Examples of substrates include: microtiter plates, test tubes, and micro-centrifuge tubes. In one example, agents that bind a polypeptide of interest can be detected by anchoring either the polypeptide of interest (e.g., any polypeptide herein) or the test agent (e.g., antibody) to a substrate (e.g., microtiter plates) and then detecting complexes of the polypeptide of interest and test agent anchored to the substrate at the end of the reaction. Where the polypeptide of interest is anchored and the test agent is not anchored, the test agent can be labeled, either directly or indirectly. In other embodiments, the polypeptide or other components of the assay maybe labeled, either directly or indirectly.

In a preferred embodiment, microtiter plates are used as the solid phase, and the anchored component can be immobilized by non-covalent or covalent attachments. Non-covalent attachments can be achieved by simply coating the solid surface with a solution of the protein and drying. In another preferred embodiment, an immobilized antibody (preferably a monoclonal antibody) specific for the polypeptide to be immobilized can be used to anchor the polypeptide to the solid surface. The surface can be prepared in advance and stored.

In another embodiment, a fusion protein (e.g., a glutathione-S-transferase fusion protein) can be provided which adds a domain that allows the polypeptides, binding agents or test agents to be bound to a matrix or other solid support. A non-immobilized component is then added to the coated surface containing the anchored component. After the reaction is complete, unreacted components are removed (e.g., by washing) and complexes anchored on the solid surface are detected. Where the non-immobilized component is pre-labeled, the detection of label immobilized on the surface indicates that the complexes were formed. Where the non-immobilized component is not pre-labeled, an indirect label can be used to detect complexes anchored on the surface, such as by using a labeled antibody specific for the non-immobilized component. The antibody can then be labeled or indirectly labeled, e.g., with an anti-Ig antibody.

Alternatively, this reaction can be conducted in a liquid phase, the reaction products separated from unreacted components, and complexes detected using, for example, an immobilized antibody specific for a polypeptide of interest or test agent to anchor the complexes formed in solution and a labeled antibody specific for the other component of the possible complex to detect anchored complexes.

In another embodiment, an assay performed in liquid phase has the pre-formed complexes of the AD polypeptides and the cellular or extracellular binding agents prepared such that either the polypeptide or the binding agents are labeled, but the signal generated from the label is eliminated or diminished due to complex formation. The addition of a test agent that competes with and displaces one of the species from the pre-formed complex results in the generation of a signal above background.

In one particular embodiment, the AD polypeptide is prepared using recombinant DNA techniques described herein and is fused to a glutathione-S-transferase (GST) gene using a fusion factor such as pGEX-5X-1, such that its binding activity is maintained in the resulting fusion product. The cellular or extracellular binding agent is purified and used to raise a monoclonal antibody, using methods routinely practiced in the art. This antibody can be labeled with the radioactive isotope 125I, for example by methods known in the art. In a substrate binding assay, the GST-AD polypeptide fusion product is anchored, for example, to glutathione-agarose beads. The cellular or extracellular binding agent is then added in the presence or absence of the test agent in a manner that allows interaction and binding to occur. At the end of the reaction period, unbound material is washed away, and the labeled monoclonal antibody can be added to the system and allowed to bind to the complexed components. The interaction between the AD polypeptide and the cellular or extracellular binding agents is detected by measuring the amount of radioactivity that remains associated with the beads. A successful inhibition of the interaction by the test agent will result in a decrease in measured radioactivity.

Alternatively, the GST bound AD polypeptide fusion product and the interactive cellular or extracellular binding agent can be mixed together in liquid in the absence of the solid glutathione-agarose beads. The test agent is added either during or after the binding agent is allowed to interact with the GST-fusion polypeptide. This mixture is then added to the glutathione-agarose beads and unbound material is washed away. The extent of inhibition of the binding agent interaction can be detected by adding the labeled antibody and comparing the radioactivity associated with the beads to that of a control reaction (e.g., lacking test agent).

The same techniques can also be employed using polypeptide fragments, derivatives, or variants that correspond to the binding domains of either the AD polypeptides (e.g., BH3) or the cellular or extracellular binding agents, or both. Binding sites can be identified and isolated using any one of a number of methods known in the art, including for example site directed mutagenesis.

Alternatively, an AD polypeptide can be anchored to a solid substrate using methods disclosed herein and allowed to interact with and bind its labeled binding agent, which has been previously treated with a proteolytic enzyme (e.g., trypsin). After washing, a short-labeled peptide comprising the binding domain (e.g., BH3) remains associated with the solid material, which can be isolated and identified by amino acid sequencing. Also, once the gene coding for the cellular or extracellular binding agent is obtained, short gene segment can be engineered to express binding fragments, which can then be tested for binding activity, purified and/or synthesized.

8. Agents that Enhance/Inhibit Genes in the AD Pathways

AD may further be prevented or treated by administering to a patient an agent that enhances or inhibits the expression or activity of genes in the associated gene pathways. Genes in the associated gene pathways are those that act upstream or downstream of the associated genomic regions in an AD-related pathway, and whose gene products may interact with, bind to, compete with, induce, enhance, or inhibit, directly or indirectly, the activity, expression, or function of genes in the associated genomic regions, or any gene whose gene products are downstream of associated genomic regions, wherein the associated genomic region induces, enhances or inhibits the expression of activity of such gene products, directly or indirectly. Genes in the pathways of LU, PVRL2, TOMM40, APOE, APOC2, APOC4, and CLPTM1 are particularly contemplated by the present invention. Also contemplated by the present invention are genes in the pathway of A2BP1, AHSG, APOE, APP, C9ORF52, CACNA1C, CKM, CTNND2, CUGBP1, DKFZP566K1924, FARS1, FGL2, FLJ14442, FLJ36760, FLJ38736, KIAA1486, KIAA1862, LAPTM4A, LNX2, LOC147468, LOC166522, LOC283867, LOC387711, LOC388110, LOC401237, LRP1B, MATN3, MGC39715, MRLC2, NCE2, PCBP3, PDE11A, PFKFB2, PPP1R12B, PSEN1, PVRL2, SEC13L1, SOX5, TGDS, TOMM40, TTLL2 and homologs thereof.

9. Potential Agents and Binding Sites

Agents that modulate the expression or activity of AD polypeptides include: nucleic acids, transcription factors, antisense nucleic acids, polypeptides, fusion proteins, PNAs, mimetics (e.g., soluble peptides or Ig-tailed fusion peptides), antibodies (e.g., monoclonal, polyclonal, humanized, anti-idiotypic, chimeric or single-chain antibodies, Fab, F(ab′)2, Fab expression library fragments, and epitope-binding fragments thereof), binding molecules, prodrugs, drugs in trials, previously approved drugs, drugs developed for indications other than AD, small and large organic or inorganic molecules, and any fragments, derivatives, variants or complements of any of the above. Such agents may be used separately or in combination.

Any of the agents herein can also serve as “lead agents” in the design and development of new pharmaceuticals. For example, sequential modification of small molecules (e.g., amino acid residue replacement with peptides, functional group replacement with peptide or non-peptide compounds) is a standard approach in the pharmaceutical industry for the development of new pharmaceuticals. Such development generally proceeds from a lead agent, which is shown to have at least some of the activity of the desired pharmaceutical. In particular, when one or more agents having at least some activity of interest are identified, structural comparison of the molecules suggest portions of the lead agents that should be conserved and portions that may be varied in the design of new candidate compounds. This embodiment also encompasses means of identifying lead agents that may be sequentially modified to produce new candidate agents for use in the treatment of AD. These new agents may be tested for therapeutic efficacy (e.g., in the cell-based or animal models described herein). This procedure may be iterated until compounds having the desired therapeutic activity and/or efficacy are identified.

10. Cell Based Assays and Animal Models

The invention provides transgenic animals having a genome comprising a transgene comprising an exogenous nucleic acid encoding a protein encoded by a gene selected from the group consisting of APOE, APP, APOC1, C9orf52, CTNND2, CUGBP1, DKFZP566K1924, FARS1, FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC388110, MGC39715, NCE2, PFKFB2, PPP1R12B, PSEN1, TGD5, and TTLL2. In some animals, the gene is selected from the group consisting of: APOC1, CACNA1C, CKM, FARS1, KIAA1486, LOC147468, LOC166522, MGC39715, NCE2, PCBP3, PDE11A, PFKFB2, PVRL2, SEC13L1, TOMM40 and TTLL2. The exogenous nucleic acid can be genomic, cDNA or minigene. The exogenous nucleic is usually from another species, particularly human, but can also be from the same species, in which case it occupies a different genomic location than the corresponding endogenous gene.

The nucleic acid preferably includes the susceptibility allele at a polymorphic site shown in FIG. 3, and more preferably a susceptibility allele shown in Table 11. The coding sequence of the gene is in operable linkage with regulatory element(s) required for its expression. Such regulatory elements can include a promoter, enhancer, one or more introns, ribosome binding site, signal sequence, polyadenylation sequence, 5′ or 3′ UTR and 5′ or 3′ flanking sequences. The regulatory sequence can be from the gene being expressed or can be heterologous. If heterologous, the regulatory sequences are usually obtained from a gene known to be expressed in the intended tissue in which the gene of the invention is to be expressed (e.g., the CNS). For example, regulatory sequences from a prion gene, PDGF or Thy-1 are suitable. The transgenic animals are disposed to develop at least one sign or symptom of an Alzheimer's related disease, particularly LOAD.

Transgenic animals having transgenes comprising susceptibility alleles of APP or PSEN1 of the invention (i.e., susceptibility alleles at SNP IDS 42182, 42256, 42263 and 201402, differ from previous transgenic animals containing these transgenes in that the susceptibility alleles are associated with LOAD rather than early-onset disease.

The invention also provides transgenic animals in which a nonhuman homolog of one of the human genes of the invention is disrupted or enhanced so as to so as to reduce, eliminate or increase its expression relative to a nontransgenic animal of the same species. Disruption can be achieved either by genetic modification of the nonhuman homolog or by functional disruption by introducing an inhibitor of expression of the gene (as discussed above) into the nonhuman animal. Enhancement of expression can be achieved either by genetically modifying the regulatory element(s) associated with an endogenous gene (e.g., introducing a stronger promoter), or by using a zinc finger protein linked to an appropriate activating domain, as discussed above.

Some transgenic animals have a plurality of transgenes respectively comprising a plurality of genes of the invention. Some transgenic animals have a plurality of disrupted nonhuman homologs of genes of the invention. Some transgenic animals combine both the presence of transgenes expressing one or more genes of the invention and one or more disruptions of nonhuman homologs of other genes of the invention.

Transgenic animals of the invention are preferably rodents, such as mice or rats, or insects, such as Drosophila. Other transgenic animals such as primates, ovines, porcines, caprines and bovines can also be used. The transgene in such animals is integrated into the genome of the animal. The transgene can be integrated in single or multiple copies. Multiple copies are generally preferred for higher expression levels. In a typical transgenic animal all germline and somatic cells include the transgene in the genome with the possible exception of a few cells that have lost the transgene as a result of spontaneous mutation or rearrangement.

For some animals, such as mice and rabbits, fertilization is performed in vivo and fertilized ova are surgically removed. In other animals, particularly bovines, it is preferable to remove ova from live or slaughterhouse animals and fertilize the ova in vitro. See DeBoer et al., WO 91/08216. Methods for culturing fertilized oocytes to the pre-implantation stage are described by Gordon et al., Methods Enzymol. 101, 414 (1984); Hogan et al., Manipulation of the Mouse Embryo: A Laboratory Manual, C.S.H.L. N.Y. (1986) (mouse embryo); Hammer et al., Nature 315, 680 (1985) (rabbit and porcine embryos); Gandolfi et al. J. Reprod. Fert. 81, 23-28 (1987); Rexroad et al., J. Anim. Sci. 66, 947-953 (1988) (ovine embryos) and Eyestone et al. J. Reprod. Fert. 85, 715-720 (1989); Camous et al., J. Reprod. Fert. 72, 779-785 (1984); and Heyman et al. Theriogenology 27, 5968 (1987) (bovine embryos) (incorporated by reference in their entirety for all purposes). Sometimes pre-implantation embryos are stored frozen for a period pending implantation. Pre-implantation embryos are transferred to the oviduct of a pseudopregnant female resulting in the birth of a transgenic or chimeric animal depending upon the stage of development when the transgene is integrated. Chimeric mammals can be bred to form true germline transgenic animals.

Alternatively, transgenes can be introduced into embryonic stem cells (ES). These cells are obtained from preimplantation embryos cultured in vitro. Bradley et al., Nature 309, 255-258 (1984) (incorporated by reference in its entirety for all purposes). Transgenes can be introduced into such cells by electroporation or microinjection. ES cells are suitable for introducing transgenes at specific chromosomal locations via homologous recombination. Transformed ES cells are combined with blastocysts from a non-human animal. The ES cells colonize the embryo and in some embryos form or contribute to the germline of the resulting chimeric animal. See Jaenisch, Science, 240, 1468-1474 (1988) (incorporated by reference in its entirety for all purposes).

Alternatively, transgenic animals can be produced by methods involving nuclear transfer. Donor nuclei are obtained from cells cultured in vitro into which a human alpha synuclein transgene is introduced using conventional methods such as Ca-phosphate transfection, microinjection or lipofection. The cells are subsequently been selected or screened for the presence of a transgene or a specific integration of a transgene (see WO 98/37183 and WO 98/39416, each incorporated by reference in their entirety for all purposes). Donor nuclei are introduced into oocytes by means of fusion, induced electrically or chemically (see any one of WO 97/07669, WO 98/30683 and WO 98/39416), or by microinjection (see WO 99/37143, incorporated by reference in its entirety for all purposes). Transplanted oocytes are subsequently cultured to develop into embryos which are subsequently implanted in the oviducts of pseudopregnant female animals, resulting in birth of transgenic offspring (see any one of WO 97/07669, WO 98/30683 and WO 98/39416).

For production of transgenic animals containing two or more transgenes, the transgenes can be introduced simultaneously using the same procedure as for a single transgene. Alternatively, the transgenes can be initially introduced into separate animals and then combined into the same genome by breeding the animals. Alternatively, a first transgenic animal is produced containing one of the transgenes. A second transgene is then introduced into fertilized ova or embryonic stem cells from that animal. Optionally, transgenes whose length would otherwise exceed about 50 kb, are constructed as overlapping fragments. Such overlapping fragments are introduced into a fertilized oocyte or embryonic stem cell simultaneously and undergo homologous recombination in vivo. See Kay et al., WO 92/03917 (incorporated by reference in its entirety for all purposes).

Nonhuman homologs of human genes of the invention can be disrupted by gene targeting. Gene targeting is a method of using homologous recombination to modify a mammalian genome, can be used to introduce changes into cultured cells. By targeting a gene of interest in embryonic stem (ES) cells, these changes can be stably introduced into the germline of laboratory animals. The gene targeting procedure is accomplished by introducing into tissue culture cells a DNA targeting construct that has a segment that can undergo homologous combination with a target locus and which also comprises an intended sequence modification (e.g., insertion, deletion, point mutation). The treated cells are then screened for accurate targeting to identify and isolate those which have been properly targeted. A common scheme to disrupt gene function by gene targeting in ES cells is to construct a targeting construct which is designed to undergo a homologous recombination with its chromosomal counterpart in the ES cell genome. The targeting constructs are typically arranged so that they insert additional sequences, such as a positive selection marker, into coding elements of the target gene, thereby functionally disrupting it. Similar procedures can also be performed on other cell types in combination with nuclear transfer. Nuclear transfer is particularly useful for creating knockouts in species other than mice for which ES cells may not be available Polejaeva et al., Nature 407, 86-90 (2000)). Breeding of nonhuman animals which are heterozygous for a null allele may be performed to produce nonhuman animals homozygous for said null allele, so-called “knockout” animals (Donehower et al. (1992) Nature 256: 215; Science 256: 1392, incorporated herein by reference).

Any of the compositions herein can be tested for their ability to prevent, ameliorate or treat symptoms associated with AD, especially dementia and memory loss. Cell-based systems can be useful for identifying agents that ameliorate symptoms associated with AD. Cell-based systems include cells that express one or more of the AD polypeptides herein and exhibit cellular phenotypes associated with resistance or susceptibility to AD. Cell-based systems include recombinant transgenic cell lines derived from animals containing one or more cells expressing one or more of the nucleic acids herein. Preferably, such cells provide a continuous cell line. Cell-based systems also include non-recombinant cell lines preferably from primary tissues of patients having AD or resistance to AD.

A cell-based system having a phenotype of AD can be exposed to an agent suspected of ameliorating phenotypic states associated with susceptibility to AD at a sufficient concentration and for a time sufficient to elicit such an amelioration response in the exposed cells. After exposure, the cells can be examined to determine whether the phenotypic states have been altered such that the phenotype has been eliminated and the cells resemble normal phenotypes or phenotypes of resistance to AD.

Animal models can be used to determine toxicity, efficacy and/or mechanism of action of the agents identified herein. Animal models for AD include both non-recombinant and recombinant transgenic animals. Non-recombinant animal models for AD include, for example, dog and murine models. Murine models can be created, for example, by administering to an animal an effective amount of alcohol or a drug to elicit a response or symptom associated with AD. Such animal models can then be exposed to an agent suspected of ameliorating AD.

Additionally, recombinant animal models exhibiting phenotypic states of AD or resistance thereto can be engineered, for example, by introducing nucleic acids associated with susceptibility or resistance, respectively. In one embodiment, an engineered sequence includes at least part of the target nucleic acid sequence and disrupts the endogenous target sequence upon integration of the engineered target gene sequence into the animal's genome. Techniques for making a transgenic animal are known in the art. For example, target gene sequences may be introduced into, and overexpressed in, the genome of the animal of interest, or, if endogenous AD-related gene sequences are present, they may either be overexpressed or, alternatively, be disrupted to underexpress or inactivate AD-related gene expression, such as described for the disruption of apoE in mice (Plump et al. (1992) Cell 71: 343-353). Other techniques include, for example, pronuclear microinjection disclosed in U.S. Pat. No. 4,873,191; retrovirus mediated gene transfer into germ-lines disclosed in Van der Putten et al., (1985) Proc. Natl. Acad. Sci. USA, 82:6148-6152; gene targeting in embryonic stem cells disclosed in Thomson et al., (1989) Cell 56:313-321; electroporation of embryos disclosed in Lo, (1983) Mol. Cell. Biol. (3) 1803-1814; and sperm-mediated gene transfer disclosed in Lavitrano et al, (1989) Cell 57:717-723; etc. For a review of such techniques, see Gordon (1989) Transgenic Animals, Intl. Rev. Cytol. 115:171-229. Nucleic acids can also be introduced into some, but not all cells of an animal to create a mosaic animal. Selective introduction into and activation of a particular cell type is discussed, for example, in Lasko et al. (1992) Proc. Natl. Acad. Sci. USA 89:6232-6236. An engineered sequence includes preferably at least part of the target nucleic acid sequence. This disrupts the endogenous target sequence upon integration of the engineered target gene sequence into the animal's genome.

In a preferred embodiment, the nucleic acids herein are used to over-express polypeptides associated with resistance to AD. In another preferred embodiment, the nucleic acids herein are used to underexpress polypeptides associated with susceptibility to AD. To overexpress a polypeptide, for example, a nucleic acid encoding the polypeptide of interest can be ligated to a regulatory sequence that can drive the expression of the polypeptide in the animal cell type of interest. Such regulatory regions are well known. In another example, a non-genic nucleic acid (e.g., an intron or a regulatory sequence) may be introduced alone to drive the production of a polypeptide of interest. To underexpress an endogenous polypeptide, a nucleic acid encoding a transcription factor that down-regulates the polypeptide or a nucleic acid that produces a variant or inactive polypeptide may be introduced into the genome of an animal such that the endogenous expression will be inactivated. In addition to, or in the alternative, a non-genic nucleic acid herein (e.g., an intron nucleic acid) may be introduced separately to override native regulatory region.

Any of the animal models disclosed herein can be used to identify agents capable of ameliorating, treating or preventing symptoms associated with susceptibility to AD. For example, animal models can be exposed to a compound suspected of exhibiting an ability to ameliorate one or more symptoms associated with AD at a sufficient concentration and for a time sufficient to elicit an ameliorating response in the exposed animal. The response of the exposed animal can be monitored by assessing change in symptoms. Any treatments that diminish one or more symptoms associated with AD or susceptibility thereto may be considered a candidate for human therapy. Dosages of test agents can be determined by deriving dose-response curves, which are well-known and commonly used in the art.

VI Pharmaceutical Compositions

Any of the agents and compositions identified herein may be produced in quantities sufficient for pharmaceutical administration and/or testing.

Pharmaceutical compositions can be formulated in accordance with the routine procedures adapted for administration to human beings. Often, pharmaceutical compositions are formulated with an acceptable carrier or excipient. See Remington's Pharmaceutical Sciences, Gennaro, A., (ed., Mack Publishing Co. 1990).

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof.

Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2 ethylamino ethanol, histidine, procaine, etc.

The pharmaceutical compositions can include, if desired, auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The pharmaceutical compositions, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides.

The pharmaceutical compositions and their physiologically acceptable salts and solvates can be formulated for administration by inhalation or insufflation (either through the mouth or the nose, or oral, buccal, parenteral, or rectal administration). For administration by inhalation, the compositions are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

For oral administration, the pharmaceutical compositions can take the form of tablets or capsules prepared by conventional means with pharmaceutically acceptable excipients such as binding agents, fillers, disintegrants, or wetting agents, sweeteners, including, pregelatinised maize starch, polyvinylpyrrolidone, hydroxypropyl methylcellulose, fillers, lactose, microcrystalline cellulose, calcium hydrogen phosphate, lubricants, magnesium stearate, talc, silica, potato starch or sodium starch glycolate, sodium lauryl sulfate, mannitol, lactose, starch, magnesium stearate, polyvinyl pyrollidone, sodium saccharine, cellulose and magnesium carbonate. The tablets can be coated by methods well known in the art. Preparations for oral administration can be suitably formulated to give controlled release of the active compound.

Liquid preparations for oral administration can take the form of solutions, syrups, or suspensions, or they can be presented as a dry product for constitution with water or other suitable vehicle before use. Such liquid preparations can be prepared by conventional means with pharmaceutically acceptable additives such as suspending agents, e.g., sorbitol syrup, cellulose derivatives, or hydrogenated edible fats; emulsifying agents, e.g., lecithin or acacia; non-aqueous vehicles, e.g., almond oil, oily esters, ethyl alcohol, or fractionated vegetable oils; and preservatives, e.g., methyl or propyl-p-hydroxybenzoates or sorbic acid. The preparations can also contain buffer salts, flavoring, coloring, and/or sweetening agents as appropriate.

In particular, the liquid preparations can be administered in a beverage. Such beverage can be alcoholic, non-alcoholic beverage or a health beverage. Such beverage may comprise one or more of the agents or compositions herein as well as, optionally, any one or more of the following: alcohol fructose, vitamins, electrolyte substitutes, caffeine, amino acids, minerals, artificial and natural sweeteners, milk or dry-milk powder and other additives and preserving agents.

Examples of vitamins that may be included are components of the vitamin B complex, such as vitamin B1, B2, B6, B12, biotin, niacin, pantothenic acid, folic acid, adenine, choline, adenosine phosphate, orotic acid, pangamic acid, carnitine, 4-aminobenzoic acid, myo-inositol, liponic acid and/or amygdaline. In the body, vitamin B1, also known as thiamin, is converted into thiamin-pyrophosphate, a coenzyme in a number of reactions in which C—C bonds are cleaved. It can also be added as thiamin hydrochloride. Vitamin B2, also known as riboflavin, is reabsorbed in the small intestines, converted into FMN (flavin mononucleotide) and, in the liver, into FAD (flavin-adenine-dinucleotide), both of which are coenzymes in redox reactions. Vitamin B6, also known as pyridoxal, pyrodoxin and pyridoxamine, is a component of pyridoxal-5-phosphate, which is a cofactor in glycogen degradation and in amino acid metabolism, e.g. as a coenzyme of decarboxylases. Preferably, this substance is admixed into the beverage in the form of pyridoxin hydrochloride. Vitamin B12, also known as cyanocobalamine, has a complex structure and is a component of cobalamine-coenzymes, with methyl-cobalamine and cobalamide, e.g., being involved in rearrangements with hydrogen migration. Biotin, also known as vitamin B7, is covalently bound to carboxylases. Niacin, also known as B3, is a generic name for nicotinic acid and nicotinamide. Niacin is a component of NAD and its phosphate, NADP, and is one of the most important hydrogen transmitters in the cell having a protective and anabolic effect on the body. Pantothenic acid, also known vitamin B3 or B5, has a precursor function for coenzyme A which assumes a central position in metabolism. Folic acid, or vitamin B9, is a component of the coenzyme tetrahydrofolate. Vitamin C may further be provided.

Preferably, the beverage composition comprises components of the vitamin B complex in the following parts by weight, based on a total of 15,000-20,000 parts by weight of the dry substance: vitamin B 1, 0.1-10 parts by weight, preferably 1 part by weight; vitamin B2, 0.1-10 parts by weight, preferably 1.5 parts by weight; vitamin B6, 0.1-10 parts by weight, preferably 1.5 parts by weight; biotin, 0.01-1 parts by weight, preferably 0.1 parts by weight; niacin, 0.1-100 parts by weight, preferably 10-30 parts by weight; pantothenic acid, 0.1-100 parts by weight, preferably 1-10 parts by weight; vitamin B 12, 0.0001-0.1 parts by weight, preferably 0.001-0.01 parts by weight; folic acid, 0.01-10 parts by weight, preferably 0.1 parts by weight, and/or vitamin C, 0.1-500 parts by weight, preferably 50 parts by weight.

It is advantageous for the beverage to comprise amino acids, in particular L-glutamine and/or L-arginine. Amino acids play an important role in the various metabolic processes of the human body. In particular, L-glutamine and L-arginine may be admixed in the beverage according to the following parts by weight, based on a total of 15,000-20,000 parts by weight of dry substance: L-arginine, 20-2,000 parts by weight, preferably 200 parts by weight; and/or L-glutamine, 10-1,000 parts by weight, preferably 100 parts by weight.

Caffeine is optionally added at 0.1-100 parts by weight, preferably 25 parts by weight, based on a total of 15,000-20,000 parts by weight.

Examples of minerals that may be used include magnesium, potassium, zinc and calcium. In particular, potassium and magnesium play an important role in metabolism and are involved in many ATP-catalyzed enzyme reactions. A mineral may be added separately, in combination, and/or in combination with other food additives, e.g. as magnesium glycerophosphate, potassium citrate (acid regulator), zinc gluconate (fruit acid) and calcium pantothenate. Minerals are preferably added at the following parts by weights, based on a total of 15,000-20,000 parts by weight of the dry substance: magnesium, 10-1,000 parts by weight, preferably 100 parts by weight; potassium 10-1,000 parts by weight, preferably 100 parts by weight; zinc, 0.1-100 parts by weight, preferably 10 parts by weight; calcium 10-1,000 parts by weight, preferably 100 parts by weight.

A tastier beverage may further include sugars and/or artificial sweeteners. Both artificial and natural sweeteners may be added to sweeten the compositions herein. Besides fructose, any other sugar may be admixed, such as glucose, galactose, lactose, etc. Artificial sweeteners include, for example, aspartame, saccharine and cyclamate as well as any other commercially available artificial sweeteners.

Furthermore, the compositions herein may comprise of further additives, in particular flavoring agents, preserving agents, coloring agents, antioxidants, electrolytes, enzymes, plant extracts, glycerolphosphates, acid regulators and/or acidifiers, in particular fruit acids.

A beverage may be carbonated or non-carbonated, and may be combined or based on liquids such as fruit juices, milk, tea, coffee, water etc. Moreover, alcohol may be admixed to the beverage herein.

The compositions can be formulated for intravenous administration. Compositions used for intravenous administration are typically solutions in sterile isotonic aqueous buffer. Where necessary, the compositions may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule indicating the quantity of active agent. Where the composition is to be administered by infusion, it can be dispensed with an infusion bottle containing sterile pharmaceutical grade water, saline or dextrose/water. Where the compositions are administered by injection, an ampule of sterile water for injection or saline can be provided so that the ingredients may be mixed prior to administration.

The compositions can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions, or emulsions in oily or aqueous vehicles, and can contain formulatory agents such as suspending, stabilizing, and/or dispersing agents. Alternatively, the active ingredient can be in powder form for constitution with a suitable vehicle, e.g., sterile pyrogen-free water, before use.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carver material, is packaged in a squeeze bottle or in admixture with a pressurized volatile, normally gaseous propellant, e.g., pressurized air.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration. Pharmaceutical packs or kits comprising one or more containers filled with one or more of the ingredients of the pharmaceutical compositions disclosed herein are also provided. Optionally, associated with such containers can be a notice in the form prescribed by a governmental agency regulating the manufacture, use or sale of pharmaceuticals or biological products, which notice reflects approval by the agency of manufacture, use of sale for human administration. The packs or kits can be labeled with information regarding mode of administration, sequence of drug administration (e.g., separately, sequentially or concurrently) or the like. The packs or kits may also include means for reminding the patient to take the therapy. The packs or kits can comprise of a single unit dosage of the combination therapy or a plurality of unit dosages. In particular, the compositions can be separated, mixed together or present in a single vial or tablet. Compositions assembled in a blister pack or other dispensing means are preferred. Unit dosages provided are preferably dependent on the pharmacodynamics of each agent and administered in FDA approved dosages in standard time courses.

VII Methods for Treatment

The agents and pharmaceutical compositions herein can be used as prophylactic or therapeutic treatment of AD. AD may result from excessive levels of certain gene products (e.g., AD polypeptides) or deficient levels of other gene products (e.g., polypeptides associated with resistance to AD).

1. Indications for Treatment

Some indications that may be used to diagnose AD include memory loss for simple things like friends' names, commonly used phone numbers, or what month it is and how to get to a familiar place; misplacement of things more often than usual; loss of train of thought when speaking; repeating things often; feeling more suspicious, cautious, or anxious; loss of interest in things that used to be enjoyable; and feeling of stress when making decisions. A more definitive indication of AD may require a biopsy.

2. Methods for Administration

The agents and pharmaceutical compositions herein can be administered separately or in combination, in an amount effective to treat an indication of interest. For example, a patient diagnosed with or afflicted by AD may be administered a therapeutically effective amount of an inhibitor of polypeptides associated with susceptibility to AD to reduce the level of activity and/or expression of such polypeptides. In the alternative, a patient diagnosed with or afflicted by an AD may be administered a therapeutically effective amount of an agonist of polypeptides associated with resistance to AD to reduce the level of activity and/or expression of such polypeptides. More preferably, a patient diagnosed with or afflicted by an AD is administered a combination treatment of both inhibitors of polypeptides associated with susceptibility to AD and agonists of polypeptides associated with resistance to AD. Such combination treatment may require lower dosages due to the synergetic effect of both compounds.

Examples of agents that may be administrated in combination with any of the compositions herein to treat or prevent AD include: cholinesterase inhibitors (e.g., galantamine, rivastigmine, donepezil, and tacrine); N-methyl D-aspartate (NMDA) antagonist (e.g., memantine); common heart drugs (e.g., ACE inhibitors), cholesterol-lowering drugs (e.g., Lipitor™ and Zocor™), galantamine hydrobromide, gingko biloba, and cholinesterase inhibitors (ChEIs) (e.g., donepezil), and other agents described above.

The agents and pharmaceutical compositions may be administered or co-administered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds and/or compositions may also be administered or co-administered in slow release dosage forms. Other suitable methods include gene therapy using rechargeable or biodegradable devices, particle acceleration devices (“gene guns”) and slow release polymeric devices. The pharmaceutical compositions herein can also be administered as part of a combinatorial therapy with other agents.

The combination of therapeutic agents and compositions may be administered by a variety of routes, and may be administered or co-administered in any conventional dosage form. Co-administration in the context of this invention is defined to mean the administration of more than one therapeutic in the course of a coordinated treatment to achieve an improved clinical outcome. Such co-administration may also be coextensive, that is, occurring during overlapping periods of time. For example, a associated genomic region antisense may be administered to a patient before, concomitantly, or after the administration of an inhibitor of AD polypeptides.

In a preferred embodiment, a pharmaceutical compound is administered orally, and more preferably is self-administered. For example, a beverage comprising one or more agents or pharmaceutical compositions may be administered to prevent, ameliorate or treat AD. The dosage of active ingredients may be based on the composition, its interaction with other compounds, or the stage of progression of AD in a patient, for example.

A patient is preferably monitored following administration of an agent of the invention for changes in a sign or symptom of the Alzheimer's related disease against which treatment or prophylaxis is being effected.

3. Gene Replacement Therapy

In another embodiment, nucleic acids can be introduced into recipient cells using techniques such as gene replacement therapy.

Preferably, one or more nucleic acids associated with resistance to AD may be inserted into appropriate cells within a patient, using vectors such as adenovirus, adeno-associated virus and retrovirus vectors. Nucleic acids can also be introduced into cells via particles, such as liposomes. Other techniques for direct administration involve stereotactic delivery of such sequences to the site of the cells in which the sequences are to be expressed.

Methods for introducing nucleic acids into mammalian cells are well known in the art. Generally, the nucleic acid is directly administered in vivo into a target cell or a transgenic mouse that expresses SP-10 promoter operably linked to a reporter gene. This can be accomplished by any methods known in the art, e.g., by constructing it as part of an appropriate nucleic acid expression vector and administering it so that it becomes intracellular, e.g., by infection using a defective or attenuated retroviral or other viral vector (U.S. Pat. No. 4,980,286), by direct injection of naked DNA, by use of microparticle bombardment (e.g., a gene gun; Biolistic, Dupont), by coating with lipids or cell-surface receptors or transfecting agents, by encapsulation in liposomes, microparticles, or microcapsules, by administering it in linkage to a peptide which is known to enter the nucleus, or by administering it in linkage to a ligand subject to receptor-mediated endocytosis (Wu and Wu, (1987) J. Biol. Chem. 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992; WO 92/22635 dated Dec. 23, 1992; WO92/20316 dated Nov. 26, 1992; WO93/14188 dated Jul. 22, 1993; WO 93/20221 dated Oct. 14, 1993).

Additional methods that may be utilized to increase or decrease the overall level of expression of an AD nucleic acid include using targeted homologous recombination methods to modify the expression characteristics of an endogenous sequence in a cell or microorganism by inserting a heterologous DNA regulatory element such that the inserted regulatory element is operatively linked with the endogenous sequence in question. Targeted homologous recombination can thus be used to activate transcription of an endogenous nucleic acid that is transcriptionally silent, (e.g., not normally expressed or expressed at very low levels), to silence the transcription of an endogenous nucleic acid that is transcriptionally active, or to enhance or decrease the expression of an endogenous sequence that is normally expressed.

Further, the overall level of expression of polypeptides associated with resistance to AD may be increased by the introduction of cells that express such polypeptides associated with resistance to AD, preferably autologous cells, into a patient at positions and in numbers which are sufficient to prevent or ameliorate symptoms or conditions associated with AD. Such cells may be either recombinant or non-recombinant. In a preferred embodiment, such cells are healthy brain cells.

When the cells to be administered are non-autologous cells, they can be administered using well-known techniques that prevent a host immune response against the introduced cells from developing. For example, the cells may be introduced in an encapsulated form that, while allowing for an exchange of components with the immediate extracellular environment, does not allow the introduced cells to be recognized by the host immune system.

The amounts of therapeutic agents or compositions to be administered can vary, according to determinations made by one of skill, but preferably are in amounts effective to create reduce or reverse AD symptoms. Treatment compositions and dosages can be specifically tailored to each situation based on an individual patient's pharmacogenomics (response to a drug), phenotype, genotype and the compositions used for treatment. Preferably, for co-administration, the total amounts are less than the total amounts for each pharmaceutical compound added together. For the slow-release dosage form, appropriate release times can vary, but preferably should last from about 1 hour to about 6 months, most preferably from about 1 week to about 4 weeks. Formulations for slow release dosage can vary as determinable by one of skill, according to the particular situation and as generally taught herein.

The LD50 (the lethal dose to 50% of the population) and the ED50 (the effective dose in 50% of the population) of a pharmaceutical composition can be determined using cell cultures or animal models following standard pharmaceutical procedures. The dose ratio of lethal and effective doses is the therapeutic index and is expressed as the ratio LD50/ED50 Compounds that exhibit large therapeutic indices are preferred. Compounds that exhibit toxic side effects can also be used, but care should be taken to design a delivery system that targets such compounds to the site of affected tissue to minimize potential damage to uninfected cells.

When using cell culture to estimate the therapeutically effective dose, the dosage of such compounds lies preferably within a range of circulating concentrations that include the ED50 with little or no toxicity. A dose can also be formulated in animal models to achieve a circulating plasma concentration range that includes the IC50 (the concentration of the test compound that achieves a half-maximal inhibition of symptoms) as determined in cell culture. Such information can be used to more accurately determine useful doses in humans. Levels in plasma can be measured, for example, by high performance liquid chromatography.

The combination of therapeutic agents may be used in the form of kits. The arrangement and construction of such kits is conventionally known. Such kits may include containers for containing the inventive combination of therapeutic agents and/or compositions, and/or other apparatus for administering the inventive combination of therapeutic agents and/or compositions.

The invention will be further described by the following non-limiting examples. The teachings of all publications cited herein are incorporated herein by reference in their entirety.

### EXAMPLES

### Example 1

### Overview of Association Study

LOAD is a devastating neurodegenerative disease, characterized by the formation of pathogenic plaques in the brain, which affects as many as 10% of people 65 or older. The disease is complex, and is likely to involve the interaction of a number of genes. Apolipoprotein E (APOE) has been reported to increase the risk of LOAD and lower the age of onset (Corder, E. H. et al. Science 261, 921-3 (1993); Farrer, L. A. et al. Jama 278, 1349-56 (1997); Saunders, A. M. et al. Neurology 43, 1467-72 (1993)). In addition, linkage peaks have been reported on chromosomes 9, 10, and 12 ( Kehoe, P. et al. Hum Mol Genet 8, 237-45 (1999); Pericak-Vance, M. A. et al. Jama 278, 1237-41 (1997); Pericak-Vance, M. A. et al. Exp Gerontol 35, 1343-52 (2000); Ertekin-Taner, N. et al. Science 290, 2303-4 (2000); Bertram, L. et al. Science 290, 2302-3 (2000); Blacker, D. et al. Hum Mol Genet 12, 23-32 (2003); Myers, A. et al. Am J Med Genet 114, 235-44 (2002)).

This Example identifies genetic loci associated with LOAD. A whole-genome association study was performed in a total of 800 unrelated case and control samples, using a dense set of SNP markers (approximately 1.5 million) that cover the entire genome. To individually screen such a large number of SNPs in all 800 samples would be prohibitive. Therefore, a two-stage approach was used. First a screen for associations using pooled sample sets was performed. Allele frequency differences between the case and control sample pools was estimated, and the estimated allele frequency differences were used to select a subset of SNPs for further evaluation. This subset, which may have contained false positives in addition to true positives, was genotyped in the individual case and control samples, and the exact allele frequency differences between the populations was calculated. SNPs showing significant association with LOAD in the original sample set were analyzed in a second sample set, to verify their association.

For Phase I our aim was to demonstrate the effectiveness of this two-stage design for identifying SNPs associated with LOAD by using a subset of the 1.5 million SNP set. Overall, we analyzed approximately 250,000 of the 1.5 million SNPs in the pooled samples. We then selected approximately 20,000 of these SNPs and genotyped them in the individual case and control samples. In addition, we genotyped almost 5,000 SNPs from candidate regions thought to contain loci associated with LOAD (including APOE).

### Example 2

### Scanning the Entire Human Genome

The entire human genome was scanned to identify common variants (and others) using microarray technology platforms such as described in U.S. Pat. No. 6,969,589; U.S. Ser. No. 10/284,444, entitled “Chromosome 21 SNPs, SNP Groups and SNP Patterns,” filed on Oct. 31, 2002, assigned to the same assignee as the present application; and U.S. Pat. No. 6,897,025, all of which are incorporated herein by reference. The microarrays are manufactured using a process adapted from semiconductor manufacturing to achieve cost effectiveness and high quality.

### Example 3

### Haplotype Blocks

Variants identified were grouped into haplotype blocks using methods disclosed in U.S. Ser. Nos. 10/106,097, entitled “Methods for Genomic Analysis”, filed Mar. 26, 2002, incorporated herein by reference. Representative variants and haplotype blocks from an entire human chromosome (chromosome 21) are disclosed in, for example, Patil, N. et al, “Blocks of Limited Haplotype Diversity Revealed by High-Resolution Scanning of Human Chromosome 21” Science 294, 1719-1723 (2001) and the associated supplemental materials, incorporated herein by reference.

### Example 4

### Samples

Four hundred unrelated case samples were obtained from individuals with late-onset Alzheimer's Disease having an apparent familial basis (i.e., each individual was from a family in which at least two members had the disease but only one member per family was included in the sample). Two hundred eighty-eight of these were obtained from the NIMH Center for Genetics Initiative. The NIMH collection contains multiplex families ascertained with two or more living related individuals with AD. Individuals were evaluated clinically, with longitudinal follow-up, and diagnoses were confirmed at autopsy. We used the following criteria to select a subset of individuals with LOAD. The individuals had to be: (1) classified as “definite” (confirmed on autopsy) or “probable” (consensus or clinical diagnosis) AD; (2) age of onset for all affected members of the family≧65; and (3) Caucasian. Only one individual (usually the proband) was chosen from each family.

We obtained an additional 112 unrelated case samples from the Department of Veterans Affairs, Seattle, Wash. These samples were selected using the same criteria as for the NIMH samples. A diagnosis of AD had been confirmed for all of these samples at autopsy, and thus all were classified as “definite” AD. Table 1 shows the characteristics of the 400 case samples used in this study.

TABLE 1
Characteristics of the 400 case samples used

| | Total | Definite | Probable | Average | Age |
|---|---|---|---|---|---|
| | samples | AD | AD | age | range |
| Female | 257 | 141 | 116 | 74.5 | 65-93 |
| Male | 143 | 63 | 80 | 74.4 | 65-90 |
| Total | 400 | 204 | 196 | | |

Four hundred cognitively-normal control samples were obtained from Group Health Cooperative, Seattle, as part of the UW/Group Health Alzheimer's Disease Patient Registry (UO1 AG 06781-18). They were selected from a larger set of community-based samples collected from Caucasian enrollees of Group Health (a Seattle Area HMO) aged 65 or older. Patients with no prior diagnosis of dementia were assessed by the Cognitive Abilities Screening Instrument (CASI), which is a 100-point scale based on the MMSE and Hasegawa DRS and 3MS tests. Patients were assessed every two years and those who continue to score more than 86 (equivalent to about 26 on the MMSE test) and did not show any symptoms of dementia were classified as controls.

### Example 5

### Assay Design for Pooled Genotyping

We designed assays for a total of 267,852 SNPs to be examined in the sample pools. These SNPs are distributed relatively evenly across the genome, with an average gap size of 11 kb. Thus, these SNPs are not focused on specific regions of the genome. We have extensive experience with this set of high-quality SNPs, all of which have minor allele frequencies of at least 10% in Caucasian populations and can be genotyped with our technology.

We used oligonucleotide arrays designed such that each SNP was interrogated by forty distinct 25 bp probes. These forty features consisted of four sets of ten features, corresponding to the forward and reverse strands of the two SNP alleles (reference and alternate). Each set of ten features consisted of two sets of five features, with offsets of −2, −1, 0, +1, and +2 bases between the center of the 25 bp probe and the SNP position. For each offset, we tiled one perfect-match feature and one mismatch feature (complement of the perfect match) at the central position of the probe. Thus, for each allele there were a total of ten perfect-match probes and ten mismatch probes. The oligonucleotide features necessary to query the 267,852 SNPs studied here were arrayed on three distinct array designs.

### Example 6

### Generation of Pools

Samples were analyzed for quality as follows: (1) concentration and volume were measured to make sure that they matched the expected values, and were adequate for the study; (2) gel electrophoresis was performed on a subset of samples to examine DNA integrity; and (3) PCR assays were performed to establish the ability of a subset of the DNA samples to be amplified.

After passing QC the samples were diluted to a concentration of 600 μg/ml, and re-quantified by PicoGreen assay. We divided the samples into a total of eight pools, four containing case samples and four containing control samples. Thus, each pool contained 100 samples, randomly selected from either the cases or controls, with each sample present in just one pool. Equimolar amounts (600 ng) of each sample were transferred into one of the eight pools robotically. Each pool was then re-quantified by PicoGreen assay and diluted to a standard concentration for use as a PCR template.

### Example 7

### Genotyping of Pooled Samples

The pools were independently amplified using multiplexed PCR with a single primer pair for each SNP. The amplified products were pooled, labeled and hybridized to the three different chip designs that together query the set of 267,852 SNPs described above. The hybridized chips were washed and stained with Cy-chrome. The hybridization of labeled sample was detected by measuring Cy-chrome fluorescence.

After removing SNP measurements that failed quality control (see below), the estimated allele frequency difference between case and control pools, termed delta p-hat, was automatically derived for each SNP from intensity ratios for hybridization to the allele-specific 25-mer features. The fluorescence intensities of the reference and alternate perfect-match features on the arrays correlate with the concentration of the corresponding SNP allele in the DNA sample. Our estimates of allele frequency, p-hat, were computed from ratios of trimmed means of intensities of the perfect-match features, after subtracting a measure of background computed from trimmed means of intensities of mismatch features. The case pool p-hats and control pool p-hats were separately averaged, and the delta p-hat was calculated. Finally, the standard error of the estimate, based on the within pool variance of the measurements, t-statistic p-value, and empirical p-values (which were obtained as rank of T_TEST_P_VALUE on each chip design divided by the total number of passing SNP measurements for each chip design) for the delta p-hat were calculated for each of the SNPs that passed the QC filters.

### Example 8

### Quality Control

The following quality control filters were applied to the data to assess the reliability of the fluorescence intensities of the features for each SNP in an array scan. Applying these filters, which are based on findings from numerous previous association studies, increases the quality of the passing SNPS, thereby reducing false-positive associations. SNP measurements were removed from consideration if they had any of the following: (1) conformance of <0.9; (2) saturated probes; and (3) signal-to-background ratio of <1.5.

The conformance of alleles was defined as the fraction of feature pairs for which the perfect-match feature is brighter than the corresponding mismatch feature. A conformance of <0.9 can indicate the absence of target DNA. Both saturated probes and low signal-to-background ratios can lead to unreliable p-hat measurements.

Due to technical problems, PCR plates containing 970 SNPs did not amplify and were therefore not analyzed in the pools. While there were no pooled data for these SNPs, they were included in the individual genotyping (see below). Table 2 shows that the pass-rate for the remaining 266882 SNPs is 93.80%, with the eight case and control pools producing consistently high pass-rates.

TABLE 2
Pooled genotyping SNP pass-rates

| Pool | Passed SNPs | Assayed SNPs | Pass-rate |
|---|---|---|---|
| case pool 1 | 253653 | 266882 | 95.04 |
| case pool 2 | 246856 | 266882 | 92.50 |
| case pool 3 | 247842 | 266882 | 92.87 |
| case pool 4 | 249290 | 266882 | 93.41 |
| average case pool | | | 93.45 |
| control pool 1 | 251434 | 266882 | 94.21 |
| control pool 2 | 252618 | 266882 | 94.66 |
| control pool 3 | 252309 | 266882 | 94.54 |
| control pool 4 | 248703 | 266882 | 93.19 |
| average control pool | | | 94.15 |
| Overall average | | | 93.80 |

### Example 9

### Selection of SNPs for Further Evaluation

SNPs were selected for further evaluation (individual genotyping) if they fell into one of the following categories:

(1) SNPs were selected if they met both of the following criteria in the pooled genotyping: (a) the corrected empirical p-values for the delta p-hat were <0.08; and

(b) measurements passed QC filters in at least two of the four case pools and at least two of the four control pools;

(2) SNPs were selected if they met both of the following criteria in the pooled genotyping: (a) the empirical p-values for the delta p-hat were <0.0015; and

(b) measurements passed QC filters in at least two of the four case pools and at least two of the four control pools;

(3) SNPs were selected if they met all three of the following criteria in the pooled genotyping: (a) measurements passed QC filters in at least three of the four case pools and at least three of the four control pools; (b) the standard error (SE) of the delta p-hat measurements is <0.04; and (c) delta p-hat >0.07;

(4) SNPs not amplified in the pooled genotyping phase;

(5) Genomic control SNPs; or

(6) SNPs from candidate regions (see Table 4 for details of regions studied)

The number of SNPs that fell into each category is given in Table 3. Note that due to the inherent experimental noise of the pooled genotyping, the majority of the category 1-3 SNPs will be false positives. True associations will be identified after genotyping this set of SNPs in individual case and control samples.

TABLE 3
Selection of SNPs by category

| Category | Number of SNPs selected | |
|---|---|---|
| 1 | 20,125 | |
| 2 | 26 | (another 350 overlapped category 1) |
| 3 | 0 | (another 2930 overlapped category 1) |
| 4 | 970 | |
| 5 | 307 | (another 4 overlapped categories 1-4) |
| 6 | 4562 | (another 317 overlapped category 1-5) |
| total | 25990 | |

TABLE 4
Additional SNPs chosen from candidate regions

| | | | | | SNPs | | |
|---|---|---|---|---|---|---|---|
| | Interval | | Start | End | picked for | SNP | |
| Chromosome | size | | position | position | genotyping | density | |
| 10 | 35 | Mb | 60,000,000 | 95,000,000 | 3656 | 1/9.6 | kb |
| 12 | 8 | Mb | 2,000,000 | 10,000,000 | 1173 | 1/6.8 | kb |
| 19 | 200 | kb | 50,000,000 | 50,200,000 | 50 | 1/4 | kb |
| total | | | | | 4879 | | |

SNPs Genotyped in Individual Samples

A total of 25,990 SNPs were individually genotyped in each of the 800 case and control samples. These included SNPs selected on the basis of the pooled genotyping results, SNPs from the candidate regions, and genomic control SNPs (see Tables 3 and 4).

High-Density Oligonucleotide Arrays

We created a new array design to genotype the selected SNPs, such that all 25,990 SNPs would be assayed using a single chip for each individual DNA sample.

Individual Genotyping of Case and Control Samples

We used multiplexed PCR to amplify the SNPs from the 800 individuals. We pooled the PCR reactions from a single individual together, and created one labeled target from each PCR pool. We hybridized the 800 individuals (targets) to oligonucleotide arrays, thereby querying each SNP once in each sample. The hybridized chips were washed and stained, and the resulting fluorescence detected as for the pooled genotyping.

Individual genotypes for each SNP were determined by clustering the intensity measurements of all samples, in the two-dimensional space defined by background-adjusted trimmed mean intensities of the perfect-match features for the reference and alternate alleles. See Hinds, D. A. et al. Matching strategies for genetic association studies in structured populations. Am J Hum Genet 74, 317-25 (2004); Hinds, D. A. et al. Application of pooled genotyping to scan candidate regions for association with HDL cholesterol levels. Human Genomics 1, 421-34 (2004); and Hinds, D. A. et al. Whole genome patterns of common DNA variation in diverse human populations. Science (Submitted). We used a K-means algorithm to assign the measurements to clusters representing the three distinct diploid genotypes that are possible: homozygous-reference, heterozygous, and homozygous-alternate. The K-means and background optimization steps were iterated until cluster membership and background estimates converged. To determine the appropriate number of genotype clusters, we repeated the analysis for 1, 2, and 3 clusters, and selected the most likely solution, considering likelihoods of the data and the cluster parameters.

Using the STRUCTURE program with the genotyping results for the 311 genomic control SNPs, we then tested for population stratification and computed the genomic control variance inflation factor. For the SNPs that passed our quality control criteria, we then computed trend test and genomic control-adjusted trend test p-values. The trend test tests for the difference in genotypes (11, 12 and 22) between cases and controls. It provides a chi-square distributed statistic that does not rely on the Hardy-Weinberg equilibrium of the SNP (contrary to a chi-square test based on allele counts rather than genotypes). The test is additive in the sense that if Allele 2 is the predisposing allele, the individual with a 12 genotype is at half the increased risk of an individual with a 22 genotype. This is in contrast to models that are recessive (only 22 is predisposed) or dominant (the increased risk of an individual with a 12 genotype is equal to the risk of a 22 individual). The genomic control-adjusted trend test was corrected using the variance inflation factor computed using genomic control markers.

Quality Control

The following quality control filters were applied to the data to assess the reliability of the fluorescence intensities of the features for each SNP in an array scan: a call-rate of 0.8, meaning that the SNP has an unambiguous genotype call in at least 80% of the samples; and a Hardy-Weinberg equilibrium p-value of >0.0001. SNP call rates were computed after discarding genotypes that obtained <0.2 score with our IG quality metric. The metric uses machine learning algorithm to approximate a probability of a genotype being discordant with outside platforms from 15 QC and SNP-property based inputs. Applying these filters, which are based on findings from numerous previous association studies, increases the quality of the passing SNPs. A total of 23,319 SNPs (90%) passed the individual genotyping quality filters, and were analyzed further.

Individual Genotyping Results: Genetic Stratification

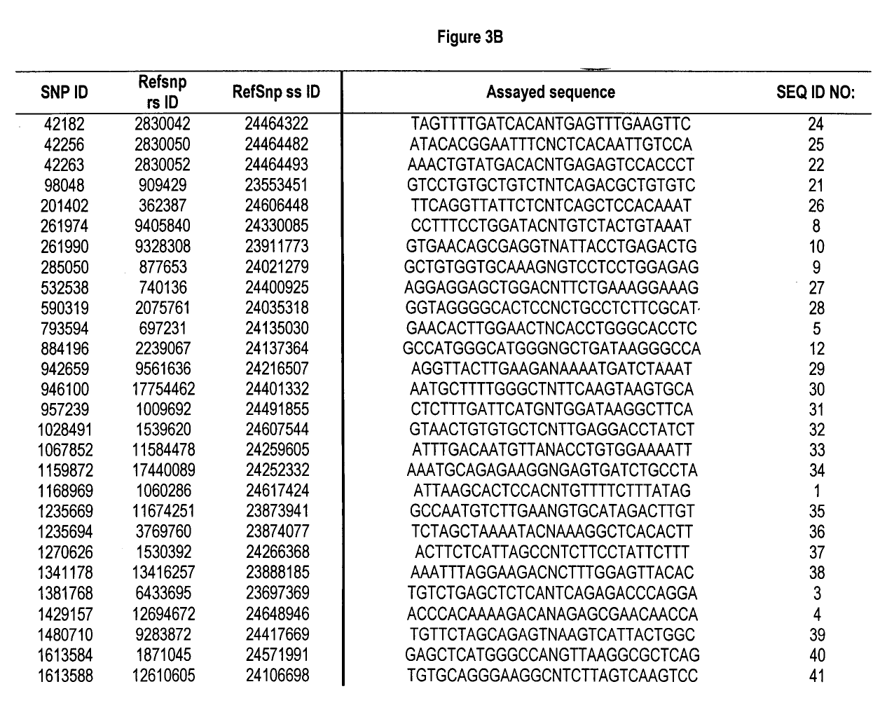

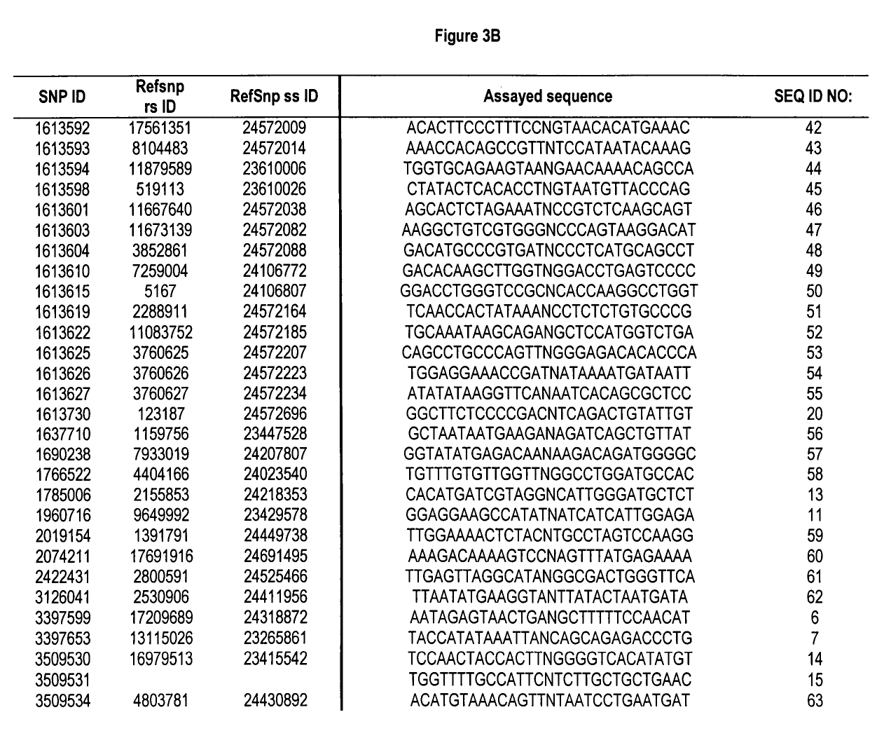

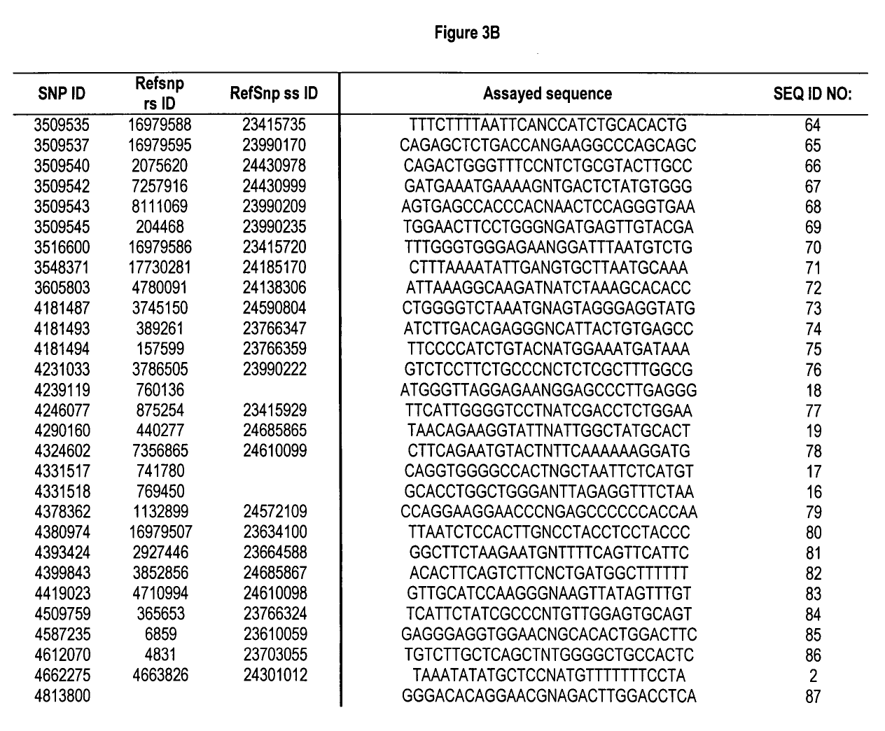

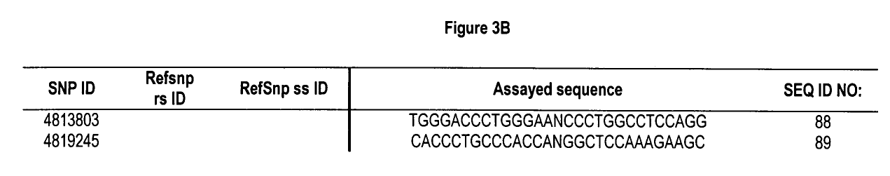

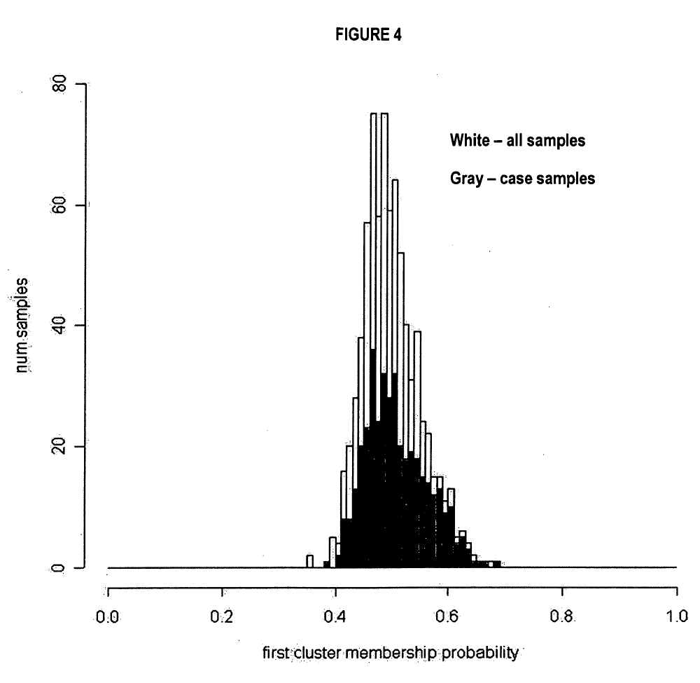

The individuals used in this study are all self-reported Caucasians, which should limit the potential for population stratification issues. However, to test for possible stratification between the case and control sample sets, we used the clustering algorithm STRUCTURE (Pritchard, J. K. & Rosenberg, N. A. Use of unlinked genetic markers to detect population stratification in association studies. Am J Hum Genet 65, 220-8 (1999)) to analyze the genotypes of 311 genomic control SNPs in the individual case and control samples. The genomic control SNPs are distributed roughly evenly across the autosomes (Hinds, D. A. et al. Matching strategies for genetic association studies in structured populations. Am J Hum Genet 74, 317-25 (2004)). FIG. 4 shows that when the samples were tested for two distinct populations, the cases and controls showed similar distributions of inferred population memberships. However, there is evidence of some difference in the distribution of the case and control individuals within the population. We used the genomic control trend test (Bacanu, S. A., Devlin, B. & Roeder, K. The power of genomic control. Am J Hum Genet 66, 1933-44 (2000)), which takes into account the average differences between case and control samples in the distribution, to correct for this.

Individual Genotyping Results

Assessing the False-Positive and False-Discovery Rates

We calculated the number of SNPs with significant trend test p-values that we would expect to find purely by chance, assuming no enrichment of large allele frequency differences in the pooling phase of the study. From the 23,319 SNPs that passed individual genotyping quality filters, we expect to find 23,319×p-value cutoff false-positives. So, for a trend test p-value of less than 1.00E-05, we expect to find 23,319×1.00E-05, or 0.23319 false-positives. As shown in Table 5, the number of observed SNPs below each of the different p-value cutoffs was well above the expected number. This indicates that the pooled genotyping did indeed enrich our SNP set for SNPs with large allele frequency differences.

TABLE 5
Number of expected and observed significant SNPs

| Trend test | | |
|---|---|---|
| p-value | Number of expected | Number of observed |
| cutoff | significant SNPs | significant SNPs |
| 1.00E−02 | 233.19 | 1117 |
| 1.00E−03 | 23.319 | 144 |
| 1.00E−05 | 0.23319 | 9 |
| 1.00E−08 | 0.00023319 | 7 |

Using the results from the 200 kb Chromosome 19 candidate region, for which 44 SNPs passed our quality filters, we estimated the false discovery rate. We calculated the false-discovery rate as the ratio of the expected number of false positives to the number of observed SNPs with significant trend test p-values below a certain cutoff. As shown in Table 6, the false-discovery rates estimated for different trend test p-value cutoffs are extremely low e.g. for SNPs with trend test p-values of less than 1.00E-08, the false discovery rate is estimated to be 6.29E-08.

TABLE 6
Individual genotyping false-discovery rates

| | | Number of | |
|---|---|---|---|
| Trend test | Number of | observed | Estimate of |
| p-value | SNPs analyzed | significant | false-discovery |
| cutoff | in the region | SNPs | rate |
| 1.00E−02 | 44 | 13 | 3.38E−02 |
| 1.00E−03 | 44 | 9 | 4.89E−03 |
| 1.00E−05 | 44 | 7 | 6.29E−05 |
| 1.00E−08 | 44 | 7 | 6.29E−08 |

Assessing the Power of Our Pooled Genotyping Method

The aim of the pooled genotyping is to enrich for significant SNPs, so that we reduce the number of SNPs requiring genotyping in each of the individual samples. We expect a large number of the SNPs selected for individual genotyping on the basis of the pooled genotyping results to be false positives. Thus it is important to show that amongst these false positives we have captured a large fraction of the SNPs that we are looking for—those with true allele frequency differences between the case and control populations. That is, is the pooled genotyping powerful enough to identify SNPs with significant allele frequency differences between the case and control pools, even if the differences are relatively small.

In this experiment we used criteria that resulted in the selection of approximately 8% of the SNPs that were genotyped in the pooled samples for follow-up genotyping in the individual samples. This was done because our previous studies had suggested that it would provide strong enough power to identify truly-associated SNPs while limiting the number of false-positives. The analysis of SNPs within the three candidate regions by both individual and pooled genotyping gave us the opportunity to actually quantify the power of the pooled genotyping in this study.

A total of 3591 SNPs were genotyped in both the pooled samples and the individual samples. The individual genotyping results showed that 1181 of these had allele frequency differences of ≧0.03, with 9 having allele frequency differences of ≧0.08 and 3 having allele frequency differences of ≧0.1 (see Table 7). Table 7 shows the percentage of the SNPs with differential allele frequencies that would be selected for individual genotyping when different selection criteria are used. As expected, the ability to detect SNPs with differences in allele frequencies using pooled genotyping depends on the size of the difference, with the smallest differences being the most difficult to identify. In addition, selecting more of the SNPs leads to more of the significant SNPs being selected, e.g. selection of 20% of the SNPs analyzed by pooled genotyping would lead to 88.89% of the SNPs with allele frequency differences of at least 0.08 being identified, whereas selection of 2% would lead to only 44.44% being identified. However, as most of the 20% will be false-positives, we have to balance the potential to identify important SNPs with the expense of genotyping large numbers of false positives. The results show that by using the criteria which resulted in the selection of 8% of the SNPs genotyped in the pools we identified approximately 56% of the SNPs with an allele frequency difference of at least 0.08 (see highlighted column in Table 7). Thus, the pooled genotyping strategy used in this study provided enough power to identify a large fraction of the SNPs with the relatively small allele frequency differences likely to be important for the complex disease of LOAD.

TABLE 7
Power of pooled genotyping method to identify SNPs with different allele frequencies

| Allele | Number | | | | | | | | | | |
|---|---|---|---|---|---|---|---|---|---|---|---|
| frequency | SNPs | | | | | | | | | | |
| difference | found by | % SNPs chosen or basis of pooled genotyping results | | | | | | | | | |
| (IG)¹ | IG² | 2% | 4% | 6% | 8% | 10% | 12% | 14% | 16% | 18% | 20% |
| ≧0.03 | 676 | 6.36% | 12.87% | 17.60% | 21.30% | 25.00% | 28.11% | 31.51% | 35.65% | 39.20% | 41.42% |
| ≧0.04 | 317 | 9.46% | 19.56% | 27.13% | 31.86% | 35.65% | 39.12% | 44.48% | 48.26% | 51.42% | 53.94% |
| ≧0.05 | 122 | 13.11% | 27.05% | 36.07% | 39.34% | 44.26% | 47.54% | 55.74% | 60.66% | 64.75% | 67.21% |
| ≧0.06 | 54 | 18.52% | 25.93% | 42.59% | 44.44% | 46.30% | 48.15% | 53.70% | 57.41% | 64.81% | 64.81% |
| ≧0.08 | 9 | 44.44% | 44.44% | 44.44% | 55.56% | 55.56% | 55.56% | 66.67% | 66.67% | 88.89% | 88.89% |
| ≧0.10 | 3 | 66.67% | 66.67% | 66.67% | 66.67% | 66.67% | 66.67% | 66.67% | 66.67% | 100.00% | 100.00% |

¹The allele frequency difference as measured by individual genotyping (IG)
²The number of SNPs found by individual genotyping (IG) to have allele frequency differences

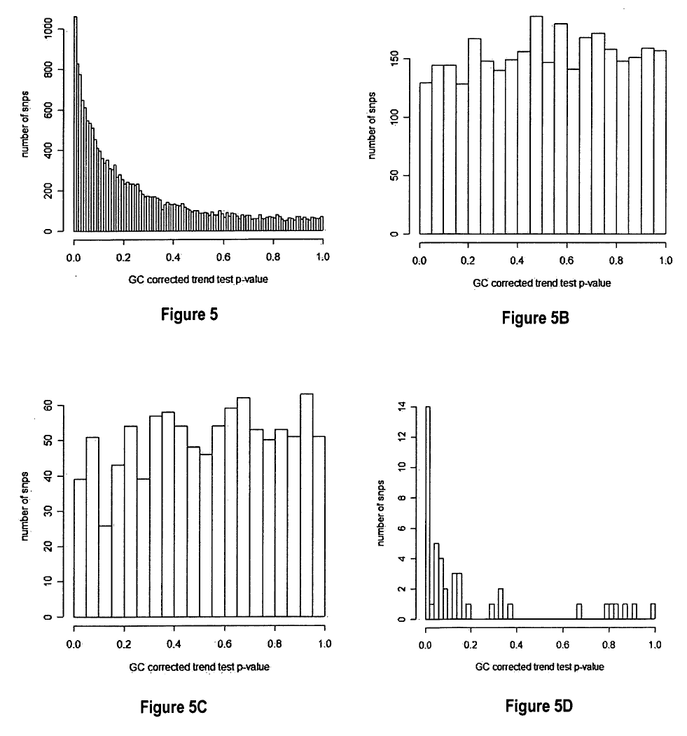

FIG. 5 shows the distribution of genomic control-corrected trend test p-values for the SNPs selected from throughout the genome by pooled genotyping (a), and the SNPs from the Chromosome 10 (b), 12 (c), and 19 (d) candidate regions. The results show that of the SNPs selected on the basis of the pooled genotyping results, there are many more than expected by chance with significant p-values. This indicates that genotyping the SNPs in the pooled samples before individual genotyping leads to an enrichment of SNPs with significant allele frequency differences, as intended. In contrast, the numbers of SNPs with significant p-values in the Chromosome 10 and 12 candidate regions are no larger than those expected by chance. For the Chromosome 19 candidate region there are a number of SNPs with highly significant p-values, as discussed in the next section.

### Example 11

### Candidate Regions

Three chosen candidate regions were examined in detail by individual genotyping, using a high density of SNPs (see Table 4).

Chromosome 19: APOE Region

A total of 44 SNPs from the 200 kb region surrounding the APOE gene on Chromosome 19 passed the individual genotyping quality control and were examined for association (23 of these were also genotyped in the pooled samples). This region has been studied in some detail by others, including Martin et al. (Martin, E. R. et al. SNPing away at complex diseases: analysis of single-nucleotide polymorphisms around APOE in Alzheimer disease. Am J Hum Genet 67, 383-94 (2000), who examined a 1.5 Mb region surrounding APOE, and found association of SNPs located within 40 kb of either side of APOE. We genotyped a total of 44 SNPs in a similar interval around the APOE gene. FIG. 1 shows the results of the individual genotyping for all of these SNPs. It also shows the pooled genotyping results for the 23 SNPs that were included in the pooled genotyping (the remaining 21 were only individually genotyped because of their location in candidate regions). Eight of the SNPs showed significant allele frequency differences between the case and control samples (highlighted in green). One of the significant SNPs is in the intron of APOE, the gene well known to contribute to LOAD, another is in an intron of the poliovirus receptor-related 2 gene (PVRL2), six more within or in close proximity with the translocase of outer mitochondrial membrane 40 homolog gene (TOMM40). Thus, we have found a number of highly-significant SNPs in the area surrounding the APOE gene.

Although the majority of the SNPs analyzed in this region were specifically included in the individual genotyping because we wanted to provide high-density coverage of this important region, the results show that all seven of the significant SNPs that were genotyped in the pooled samples would have been selected on the basis of the pooled genotyping results alone. Only one of the significant SNPs (rs16979513) was not included in the pooled genotyping screen. The gene containing this SNP, TOMM40, was identified by other significant SNPs that were genotyped in the pools. These results highlight the success of our two-stage association study strategy.

Chromosomes 10 and 12

One of the SNPs those for chromosome 12 (rsID 2239067) was found to have resistance and susceptibility alleles associated with resistance or susceptibility to LOAD.

SNPs Associated with LOAD

Using a genomic control-adjusted trend test p-value cutoff of 0.0005, a total of 53 of the 23,319 passing SNPs passed this cutoff. We do not precisely know the expected number of SNPs that should pass this criterion, as the pooling significantly enriched for large allele frequency differences. However, this set of SNPs is likely enriched for true positive associations.

In addition to the above-mentioned SNPs in the APOE region of chromosome 19, three of these 53 SNPs are in the amyloid beta (A4) precursor protein (APP) gene and one is in the presenilin 1 (PSEN1) gene. Although mutations in both of these genes are known to cause the rare early-onset form of Alzheimer's disease (Bertram & Tanzi, Hum Mol Genet 13 Spec No 1, R135-41 (2004)), there has been no previous evidence of an association between either of these genes and the common late-onset form of Alzheimer's disease. These genes were not in the candidate regions we studied, and were instead identified by the two-stage approach in which : pooled genotyping identified SNPs likely to be associated with the trait, followed by individual genotyping to identify true associations.

Significance

We have shown that our two-stage whole-genome association approach for identifying genes associated with LOAD has enough power to identify a large fraction of the SNPs with small allele-frequency differences between our case and control samples. We have identified a number of SNPs that show large differences in allele frequency between the case and control samples. Two genes near these SNPs, APP and PSEN1, are particularly exciting, as they are known to be responsible for rare cases of early-onset Alzheimer's disease but not LOAD.

### Example 12

Both the pooled genotyping and individual genotyping phases of this study involved amplification of samples by short-range PCR. For pooled genotyping, each pool was subjected to a plurality of multiplex (>100-plex) short-range PCR reactions using primers designed to amplify genomic DNA containing 267,852 SNPs. For individual genotyping, a sample from each individual was subjected to a plurality of multiplex (>100-plex), short-range PCRs using primers designed to amplify genomic DNA containing approximately 20,000 potentially associated SNPs that were identified in the pooled genotyping methodology as well as almost 5,000 SNPs from candidate regions thought to contain loci associated with LOAD. The PCRs were performed in 384-well plates containing DNA template (10 ng) and PCR cocktail (1.47 μl 10× AK2 buffer (0.5M Trizma, 0.14M ammonium sulfate, and 27 mM MgCl2), 0.03M tricine, 0.671 μl MasterAmp 10× PCR Enhancer (Epicentre, Madison, Wis.), 3.9% DMSO, 0.05M KCl, dNTPs (0.54 mM each), PCR primers (0.42 pmol/μl/primer), and ˜2× Titanium Taq polymerase (BD Biosciences, Palo Alto, Calif.)). The PCR plates were sealed prior to PCR. Short-range PCR was performed for approximately three hours. The thermocycler block was allowed to reach 90° C. before the PCR plates were placed in the thermocycler. The thermocycler program used for short-range PCR is identified in Table 8:

TABLE 8
Thermocycler program used for short-range PCR

| Step | Action |
|---|---|
| 1 | Incubate at 96° C. for 5 min |
| 2 | Incubate at 96° C. for 2 seconds |
| 3 | Incubate at 53° C. (−0.5° C./cycle) for 2 minutes |
| 4 | Go to [step] “2” (for 54 subsequent cycles) |
| 5 | Incubate at 50° C. for 15 minutes |
| 6 | Hold at 4° C. |

Once the PCR was complete, the plates were removed from the thermocycler and were pooled as described infra. (At this point, the plates could also have been stored at −20° C. for an extended period, if so desired.)

PCR plates containing amplified sample were spun at 1000 r.p.m. for 15 seconds in a table-top Sorvall centrifuge. Amplified samples from a single individual corresponding to a single chip (microarray) design were pooled together. The pooled samples were then arrayed into 96-well plates and quantified using PicoGreen reagent (Molecular Probes, Inc., Eugene, Oreg.) and a SpectraFluor Tecan Plate Reader (Tecan Group Ltd., Maennedorf, Switzerland). Amplified samples that contained less than 100 ng/μl were deemed to have failed PCR and were not analyzed further.

### Example 13

Post-PCR pools were subjected to treatment with shrimp alkaline phosphatase (SAP). Each treatment was performed in a well of a 96-well plate and contained 8 μg amplified sample, 5U SAP (Promega, Madison, Wis.), and ˜1× One Phor All buffer Plus (Amersham Biosciences, Buckinghamshire, England) in a total volume of 100 μl. The reaction mixture was incubated at 37° C. for 30 minutes, 80° C. for 20 minutes, and then cooled to 4° C. The SAP-treated samples were then labeled with biotin. (At this point, the SAP-treated sample could be stored overnight at −20° C. prior to biotin-labeling.)

### Example 14

The SAP-treated pools were labeled with biotin. Each labeling reaction was performed in one well of a 96-well plate and contained the 100 μl volume of the SAP-treated pool plus 3 μl of 0.5mM biotin d/dd-UTP and 800U of recombinant TdT. The plate was sealed, vortexed briefly, and centrifuged at 1000 r.p.m. for 15 seconds in a table-top Sorvall centrifuge. The plate was placed in a thermocycler and incubated at 37° C. for 90 minutes, 99° C. for 10 minutes, and then cooled to 4° C. The biotin-labeled pools were hybridized to microarrays on the same day as they were labeled.

### Example 15

Hybridization buffer (1.5M TMACL (tetramethylammonium chloride), 5 mM Tris (pH 7.8 or 8.0), 0.005% Triton X-100, 26 pM b-948 control oligo (Genset, La Jolla, Calif.), and 0.05 mg/ml HS (herring sperm) DNA) was prewarmed at 60° C. for a minimum of 30 minutes. Microarrays (e.g., chips) were prewarmed at 50° C. in a hybridization oven for approximately 30 minutes. 195 μl of hybridization buffer was added to each well of a 96-well plate that was prewarmed at 60° C. for a minimum of 30 minutes, and the plate (“hybridization plate”) was sealed and returned to the heat block. The 96-well plate containing the labeled sample was centrifuged at 1000 r.p.m. for 15 seconds in a table-top Sorvall centrifuge prior to heating the plate at 99° C. for 10 minutes and subsequently cooling the plate to 60° C. (for no more than 5 minutes) to denature the labeled sample. Once the denaturation is complete, the denatured samples (105 μl) were transferred to wells on the hybridization plate containing the 195 μl aliquots of hybridization buffer, and were mixed by pipetting the solution up and down twice. The hybridization plates were resealed and returned to the 60° C. heat block.

The mixture containing the denatured samples and hybridization buffer was transferred to a prewarmed microarray. The array was sealed, returned to the 50° C. hybridization oven, and rotated at 20 r.p.m. overnight (14-19 hours). After the overnight incubation, the array was stained, washed and scanned as described below.

### Example 16

After incubation (i.e., hybridization), the microarray was removed from the hybridization oven and the sample was removed and stored at −20° C. Then, the microarray was washed 1-2× with 200 μl of 1× MES/0.01% Triton X-100. The microarray was inverted several times to ensure that the wash solution moved freely over the surface of the microarray prior to removing the wash solution by vacuum suction.

Next, 200 μl of the “First Stain Solution” (174 μl of 1× MES/0.01% Triton X-100, 25 μl of 20 mg/ml of acetylated BSA, and 1 μl of 1 mg/ml streptavidin) was added to each microarray. The microarray was inverted several times to ensure that the First Stain Solution moved freely over the surface of the microarray. Then, the microarray was rotated at 25 r.p.m. for 15 minutes at room temperature. Next, the microarray was washed with 1× MES/0.01% Triton X-100 wash solution in a Perlegen RevD Fluidics Station. When the wash was finished the microarray was removed from the fluidics station and the 1× MES/0.01% Triton X-100 wash solution was removed by vacuum suction.

Next, 200 μl of the “Second Stain Solution” (175 μl of 1× MES/0.01% Triton X-100, 25 μl of 20 mg/ml acetylated BSA, and 0.5 μl of 0.5 mg/ml biotinylated anti-streptavidin) was added to each microarray. The microarray was inverted several times to ensure that the Second Stain Solution moved freely over the surface of the microarray. Then, the microarray was rotated at 25 r.p.m. for 15 minutes at room temperature. Next, the microarray was washed with 1× MES/0.01% Triton X-100 wash solution in a RevD Fluidics Station. When the wash was finished the microarray was removed from the fluidics station and the 1× MES/0.01% Triton X-100 wash solution was removed by vacuum suction.

Then, 200 μl of the “Third Stain Solution” (174 μl of 1× MES/0.01% Triton X-100, 25 μl of 20 mg/ml acetylated BSA, and 1 μl of 0.2 mg/ml streptavidin Cy-chrome) was added to each microarray. The microarray was inverted several times to ensure that the Third Stain Solution moved freely over the surface of the microarray. Then, the microarray was rotated at 25 r.p.m. for 15 minutes at room temperature. Next, the microarray was washed with 1× MES/0.01% Triton X-100 wash solution in a RevD Fluidics Station. When the wash was finished the microarray was removed from the fluidics station and the 1× MES/0.01% Triton X-100 wash solution was removed by vacuum suction.

Then, a wash solution of 6×SSPE/0.01% Triton X-100 was added to the microarray. The microarray was inverted several times to ensure that the 6×SSPE/0.01% Triton X-100 moved freely over the surface of the microarray before it was removed by vacuum suction. Next, a wash solution of 0.2×SSPE/0.005% Triton X-100 that had been prewarmed to 37° C. was added to the microarray, which was then incubated at 37° C. for 30 minutes. The 0.2×SSPE/0.005% Triton X-100 was removed by vacuum suction and a solution of 1× MES/0.01% Triton X-100 was added to the microarray. The microarray was then inverted several times before the 1× MES/0.01% Triton X-100 was removed by vacuum suction. Finally, fresh 1× MES/0.01% Triton X-100 was added to the microarray, which was wrapped in foil prior to storage at 4° C. or scanning of the microarray.

### Example 17

On the same days the microarrays were stained and washed, they were scanned using an arc scanner. After scanning, the microarrays were removed from the scanner, wrapped in foil and stored at 4° C. The scan files generated by the scanner were then analyzed by software programs designed to interpret intensity data from microarrays. For the pooled genotyping, this software allowed discrimination of hybridization patterns that distinguished the case pools from the control pools. The data were analyzed according to the methods disclosed in the following U.S. patent applications, all of which are assigned to the assignee of the present applications: U.S. provisional patent application No. 60/460,329, filed on Apr. 3, 2003, entitled “Apparatus and Methods for Analyzing and Characterizing Nucleic Acid Sequences”; and U.S. patent application Ser. No. 10/768,788, filed Jan. 30, 2004, entitled “Apparatus and Methods for Analyzing and Characterizing Nucleic Acid Sequences”. Nucleic acids that were identified as strongly associated with the case or control group based on the pooled genotyping analysis were reanalyzed by genotyping individual samples for those potentially associated nucleic acids, as described below. As such, individual genotyping was performed on approximately 30,000 (˜2%) of the original 1.7 million SNPs.

For the individual genotyping, the software assigned genotypes at each SNP position for each individual in the case and control groups. The data were analyzed according to the methods disclosed in the following U.S. patent applications, all of which are assigned to the assignee of the present applications: U.S. patent application Ser. No. 10/351,973, filed Jan. 27, 2003, entitled “Apparatus and Methods for Determining Individual Genotypes”; and U.S. patent application Ser. No. 10/786,475, filed Feb. 24, 2004, entitled “Improvements to Analysis Methods for Individual Genotyping.”

### Example 18

In this Example, an additional 433 cases and 473 controls (“replication samples”) were individually genotyped at the same 25,990 SNPs, and these genotypes along with the individual genotypes of the original 400 cases and 400 controls were used to compute a final set of delta P values. These additional samples come from two sets of replication samples. The first set consisted of 222 cases (non-familial LOAD patients) and 191 controls. These were clinic-based cases and controls. All controls were evaluated using a neuropsychological battery of tests. The frequency of evaluation of the controls depended on their age. Younger controls (60-70) were seen every 3 years, 70-80, every other year, and >80 every year. The second set of replication samples consisted of 211 familial LOAD cases and 282 controls, selected in the same way as were the original 400 cases and 400 controls.

Table 11 column 1 shows chromosome location, columns 2-4 shows polymorphic site position as defined above. Column 5 (susceptibility allele) shows the nucleotide occupying a polymorphic site that associates with susceptibility to AD, and column 6 (resistance allele) shows the nucleotide occupying a polymorphic site that associates with resistance to AD. Column 6 shows a human genomic nucleotide segment of about 30 nucleotides including a polymorphic site represented in IUPAC-IUB ambiguity code.

TABLE 11

Suscepti-

| | | | | |
|---|---|---|---|---|
| Genomic Location | dbSNP Annotation | Resistance | bility | SNP Assay with ambiguity code |

| | | | | | | |
|---|---|---|---|---|---|---|
| 3 | Position | ssID | rsID | Allele | Allele | at SNP position |

| | | | | | | |
|---|---|---|---|---|---|---|
| 1 | 203638695 | 24617424 | 1060286 | G | A | ATTAAGCACTCCACRTGTTTTCTTTATAG |
| 2 | 238690120 | 24301012 | 4663826 | C | T | TAAATATATGCTCCYATGTTTTTTTCCTA |
| 2 | 178490014 | 23697369 | 6433695 | C | T | TGTCTGAGCTCTCAYTCAGAGACCCAGGA |
| 2 | 226328835 | 24648946 | 12694672 | T | G | ACCCACAAAAGACAKAGAGCGAACAACCA |
| 3 | 10321421 | 24135030 | 697231 | A | G | GAACACTTGGAACTRCACCTGGGCACCTC |
| 4 | 10370042 | 24318872 | 17209689 | C | T | AATAGAGTAACTGAYGCTTTTTCCAACAT |
| 4 | 10346315 | 23265861 | 13115026 | G | A | TACCATATAAATTARCAGCAGAGACCCTG |
| 6 | 5452181 | 24330085 | 9405840 | A | G | CCTTTCCTGGATACRTGTCTACTGTAAAT |
| 6 | 167724102 | 24021279 | 877653 | C | T | GCTGTGGTGCAAAGYGTCCTCCTGGAGAG |
| 6 | 5458490 | 23911773 | 9328308 | T | C | GTGAACAGCGAGGTYATTACCTGAGACTG |
| 8 | 101691287 | 23429578 | 9649992 | G | C | GGAGGAAGCCATATSATCATCATTGGAGA |
| 12 | 2404255 | 24137364 | 2239067 | C | T | GCCATGGGCATGGGYGCTGATAAGGGCCA |
| 18 | 20851097 | 24218353 | 2155853 | A | G | CACATGATCGTAGGRCATTGGGATGCTCT |
| 19 | 50087984 | 23415542 | 16979513 | T | C | TCCAACTACCACTTYGGGGTCACATATGT |
| 19 | 50099628 | n/a | 7259620 | G | A | TGGTTTTGCCATTCRTCTTGCTGCTGAAC |
| 19 | 50102284 | n/a | 769450 | G | A | GCACCTGGCTGGGARTTAGAGGTTTCTAA |
| 19 | 50096271 | n/a | 741780 | T | C | CAGGTGGGGCCACTYGCTAATTCTCATGT |
| 19 | 50095698 | n/a | 760136 | A | G | ATGGGTTAGGAGAARGGAGCCCTTGAGGG |
| 19 | 50053064 | 24685865 | 440277 | G | A | TAACAGAAGGTATTRATTGGCTATGCACT |
| 19 | 50522787 | 24572696 | 123187 | G | A | GGCTTCTCCCCGACRTCAGACTGTATTGT |
| 21 | 46117896 | 23553451 | 909429 | C | T | GTCCTGTGCTGTCTYTCAGACGCTGTGTC |
| 21 | 26370641 | 24464322 | 2830042 | T | C | TAGTTTTGATCACAYTGAGTTTGAAGTTC |
| 21 | 26387468 | 24464493 | 2830052 | T | C | AAACTGTATGACACYTGAGAGTCCACCCT |

Table 12 columns 1 and 3 provide references for a SNP position. Table 12, column 2 provides the chromosome containing a SN. Columns 4, 6, and 8 are different statistical analyses used to determine significance of the delta P values. Column 8 lists p-values from the Cochran-Armitage trend test (Freidlin et al., Human Heredity 2002;53:146-152. Column 4 lists p-values from the trend test that have been corrected for population stratification. Column 6 lists Chi-squared p-values. Column 7 lists Hardy-Weinberg Equilibrium chi-squared p-values computed from the allele frequencies of the controls. Column 5 lists delta P values (the difference in frequency of a reference between cases and controls.

TABLE 12

| | | | strat | | Chi squared | HWE | total trend |
|---|---|---|---|---|---|---|---|
| rsID | Chr | Position | adj GC p | Δp | p-value | cont | p-value |
| 16979513 | 19 | 50087984 | 3.67E−44 | −0.223742 | 7.74156E−51 | 0.49934 | 3.805E−53 |
| 7259620 | 19 | 50099628 | 3.19E−20 | 0.164224 | 5.67055E−22 | 0.74092 | 3.297E−24 |
| 769450 | 19 | 50102284 | 7.59E−20 | 0.163803 | 2.29165E−21 | 0.6522 | 6.368E−24 |
| 741780 | 19 | 50096271 | 1.77E−18 | 0.158489 | 1.11043E−20 | 0.84517 | 4.4E−22 |
| 760136 | 19 | 50095698 | 4.65E−17 | 0.153685 | 1.90655E−18 | 0.88043 | 1.776E−20 |
| 440277 | 19 | 50053064 | 1.91E−07 | 0.08833 | 3.945E−07 | 0.99774 | 9.397E−09 |
| 9405840 | 6 | 5452181 | 5E−05 | 0.074632 | 0.000142566 | 0.99581 | 8.148E−06 |
| 4663826 | 2 | 238690120 | 7.27E−05 | −0.059356 | 0.00042975 | 0.0168 | 1.642E−05 |
| 877653 | 6 | 167724102 | 0.000115 | 0.066769 | 0.000312813 | 0.3602 | 2.232E−05 |
| 9328308 | 6 | 5458490 | 0.00016 | 0.060584 | 0.000808952 | 0.76675 | 3.355E−05 |
| 697231 | 3 | 10321421 | 0.000291 | −0.044136 | 0.000941046 | 0.99998 | 6.61E−05 |
| 9649992 | 8 | 101691287 | 0.000345 | −0.066439 | 0.000976789 | 0.89277 | 7.811E−05 |
| 17209689 | 4 | 10370042 | 0.00047 | 0.061903 | 0.002265812 | 0.95435 | 0.0001248 |
| 6433695 | 2 | 178490014 | 0.000618 | −0.047657 | 0.002495847 | 0.99929 | 0.0001685 |
| 2155853 | 18 | 20851097 | 0.000917 | −0.06201 | 0.001519867 | 0.00093 | 0.0002559 |
| 123187 | 19 | 50522787 | 0.000919 | 0.060999 | 0.003299686 | 0.50252 | 0.0002882 |
| 2239067 | 12 | 2404255 | 0.000998 | 0.06542 | 0.003684628 | 0.06914 | 0.0003759 |
| 12694672 | 2 | 226328835 | 0.001323 | −0.049821 | 0.00626904 | 0.60889 | 0.0004553 |
| 13115026 | 4 | 10346315 | 0.001408 | 0.055713 | 0.00621134 | 0.99404 | 0.0004416 |
| 909429 | 21 | 46117896 | 0.002455 | −0.040988 | 0.011848856 | 0.77962 | 0.0008716 |
| 1060286 | 1 | 203638695 | 0.002546 | 0.055293 | 0.011901171 | 0.85817 | 0.0009274 |
| 2830042 | 21 | 26370641 | 0.002701 | 0.027941 | 0.012537332 | 0.62983 | 0.0010321 |
| 2830052 | 21 | 26387468 | 0.002817 | −0.047293 | 0.014113395 | 0.91462 | 0.0010632 |

Table 13 lists shows the identity of the reference allele used in calculating the delta p values shown in Table 12.

TABLE 13

| | | |
|---|---|---|
| Genomic Location | | Reference Allele Frequency |
| (NCBI Build 35) | Reference | Difference (ref freq in |

| Chr | Position | allele | cases − ref freq in controls) |
|---|---|---|---|
| 1 | 203638695 | G | 0.055293455 |
| 2 | 238690120 | T | −0.059355609 |
| 2 | 178490014 | T | −0.047656762 |
| 2 | 226328835 | G | −0.049821109 |
| 3 | 10321421 | G | −0.044135983 |
| 4 | 10370042 | C | 0.061902942 |
| 4 | 10346315 | G | 0.055712619 |
| 6 | 5452181 | A | 0.074631617 |
| 6 | 167724102 | C | 0.066769264 |
| 6 | 5458490 | T | 0.060584148 |
| 8 | 101691287 | C | −0.066438845 |
| 12 | 2404255 | C | 0.065419501 |
| 18 | 20851097 | G | −0.06201012 |
| 19 | 50087984 | C | −0.223741981 |
| 19 | 50099628 | G | 0.164224473 |
| 19 | 50102284 | G | 0.163802546 |
| 19 | 50096271 | T | 0.158488954 |
| 19 | 50095698 | A | 0.15368548 |
| 19 | 50053064 | G | 0.088330411 |
| 19 | 50522787 | G | 0.060999245 |
| 21 | 46117896 | T | −0.040988457 |
| 21 | 26370641 | T | 0.027941359 |
| 21 | 26387468 | C | −0.047292708 |

1. A method of polymorphic profiling an individual comprising: determining a polymorphic profile in at least two but no more than 1000 different haplotype blocks, at least two of the haplotype blocks including a gene selected from the group consisting of APOE, APP, APOC1, CACNA1C, CKM, FARS1, KIAA1486, LOC147468, LOC166522, MGC39715, NCE2, PCBP3, PDE11A, PFKFB2, PVRL2, SEC13L1, TOMM40 and TTLL2.

2. The method of claim 1, wherein the polymorphic profile in the at least two haplotype blocks is determined at polymorphic sites in or within 10 kb of the at least two genes selected from the group.

3. The method of claim 1, wherein the polymorphic profile is determined in at least two genes selected from the group.

4. The method of claim 1, further comprising determining the total number of resistance and susceptibility alleles in the polymorphic profile, whereby the ratio of susceptibility alleles to resistance alleles provides an indication of whether the individual has or is at risk of Alzheimer's disease.

5. The method of claim 4, wherein the polymorphic profile is determined in or within 10 kb of at least ten genes selected from the group, and presence of at least twenty susceptibility and resistance alleles is determined, and a ratio of resistance to susceptibility alleles of less than fifty percent is an indication that the individual does not have Alzheimer's disease.

6. The method of claim 1, wherein the polymorphic profile is determined in an individual having a symptom of, or known susceptibility to, Alzheimer's disease.

7. The method of claim 1, wherein the at least two haplotype blocks do not include APOE and APP.

8. The method of claim 1, wherein the at least two haplotype blocks each comprise at least one gene selected from the group consisting of CACNA1C, CKM, FARS1, KIAA1486, LOC147468, LOC166522, MGC39715, NCE2, PCBP3, PDE11A, PFKFB2, SEC13L1, and TTLL2.

9. The method of claim 1, wherein the method determines the polymorphic profile in at least ten haplotype blocks, each including a different gene selected from the group.

10. The method of claim 1, wherein the polymorphic profile is determined in at least two and no more than 50 different haplotype blocks.

11. The method of claim 1, further comprising selecting a treatment or prophylactic regime for an Alzheimer's related disease based on the polymorphic profile.

12. A method of diagnosing or prognosticating Alzheimer's disease in a subject, comprising:
determining a polymorphic profile in a haplotype block of a subject including gene selected from the group consisting of APOC1, CACNA1C, CKM, FARS1, KIAA1486, LOC147468, LOC166522, MGC39715, NCE2, PCBP3, PDE11A, PFKFB2, PVRL2, SEC13L1, TOMM40 and TTLL2.

13. A method of diagnosing or prognosticating late-onset Alzheimer's disease, comprising
determining a polymorphic profile in a haplotype block that includes a gene selected from the group consisting of APP, APOC1, CACNA1C, CKM, FARS1, KIAA1486, LOC147468, LOC166522, MGC39715, NCE2, PCBP3, PDE11A, PFKFB2, PVRL2, SEC13L1, TOMM40 and TTLL2.

14. The method of claim 13, wherein the determining determines presence of a susceptibility allele shown in Table 11 or in linkage disequilibrium therewith, the susceptibility allele indicating presence or susceptibility to the late-onset Alzheimer's disease.

15. A method of diagnosing or prognosticating an Alzheimer's related disease in a patient, comprising:
determining presence of at least one susceptibility allele shown in Table 11 or in linkage disequilibrium therewith, the presence of the susceptibility allele indicating presence or susceptibility to the Alzheimer's related disease.

16. The method of claim 15, wherein the method determines presence of at least one susceptibility allele shown in Table 11.

17. The method of claim 15, provided the determining determines at least one susceptibility allele not in or within 40 kb of a gene selected from the group consisting of APP or APOE.

18. The method of claim 15, further comprising informing the patient or a relative thereof of presence or susceptibility to an Alzheimer's related disease.

19. The method of claim 15, further comprising performing a secondary test for an Alzheimer's related disease.

20. The method of claim 19, wherein the secondary test comprises determining mental activity by a psychometric measure.

21. The method of claim 19, wherein the secondary test comprises taking a biopsy.

22. The method of claim 15, further comprising administering a regime effective to treat or effect prophylaxis of an Alzheimer's related disease.

23. The method of claim 15, further provided the determining determines at least one susceptibility allele not in or within 40 kb of TOMM40 or APOC 1.

24. The method of claim 15, further provided the determining determines at least one susceptibility allele not in or within 40 kb of LU, PVRL2, TOMM40, APOE, APOC1, APOC4, APOC2, or CLPTM1.

25. The method of claim 15, wherein the determining determines presence of at least 5 susceptibility alleles in at least five different genes selected from the group consisting of APOE, APP, APOC1, CACNA1C, CKM, FARS1, KIAA1486, LOC147468, LOC166522, MGC39715, NCE2, PCBP3, PDE11A, PFKFB2, PVRL2, SEC13L1, TOMM40, and TTLL2.

26. The method of claim 15, wherein the determining determines presence of at least 10 susceptibility alleles in at least ten different genes selected from the group consisting of APOE, APP, APOC1, CACNA1C, CKM, FARS1, KIAA1486, LOC147468, LOC166522, MGC39715, NCE2, PCBP3, PDE11A, PFKFB2, PVRL2, SEC13L1, TOMM40, and TTLL2.

27. The method of claim 26, wherein the at least 10 susceptibility alleles are alleles shown Table 11.

28-59. (canceled)

60. A method of excluding an individual from a clinical trial to test a drug for treatment or prophylaxis of Alzheimer's disease, comprising
determining a polymorphic profile in an individual presenting symptoms resembling Alzheimer's disease in or within 10 kb of a plurality of genes selected from the group consisting of APOE, APP, APOC1, CACNA1C, CKM, FARS1, KIAA1486, LOC147468, LOC166522, MGC39715, NCE2, PCBP3, PDE11A, PFKFB2, PVRL2, SEC13L1, TOMM40 and TTLL2; and
determining the total number of resistance and susceptibility alleles at each locus in the polymorphic profile, wherein a high ratio of resistance to susceptibility alleles is an indication the individual should be excluded from the clinical trial.

61. A method of polymorphic profiling an individual comprising: determining a polymorphic profile in at least two but no more than 1000 different haplotype blocks, at least two of the haplotype blocks including a gene selected from the group consisting of APOE, APP, APOC1, C9orf52, CTNND2, CUGBP1, DKFZP566K1924, FARS1, FGL2, FLJ14442, FLJ36760, KIAA1486, KIAA1862, LNX2, LOC147468, LOC283867, LOC401237, LRP1B, MATN3, MRLC2, PCBP3, PDE11A, PVRL2, SEC13L1, SOX5, TOMM40, AHSG, CAGNA1C, CKM, FLJ38736, LAPTM4A, LOC166522, LOC387711, LOC388110, MGC39715, NCE2, PFKFB2, PPP1R12B, PSEN1, TGD5, and TTLL2.

62-131. (canceled)

132. A method of effecting treatment or prophylaxis of an Alzheimer's related disease, comprising:
administering to a patient an effective amount of an agent that modulates the activity or expression of a protein encoded by a gene selected from the group consisting of APOC1, CACNA1C, CKM, FARS1, KIAA1486, LOC147468, LOC166522, MGC39715, NCE2, PCBP3, PDE11A, PFKFB2, PVRL2, SEC13L1, TOMM40, and TTLL2.
